SEQUENCE LISTING (23 sequences)

SEQ ID NO: 1 - protein, 887 aa, Artificial Sequence
  Truncated lactase fragment
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKT

SEQ ID NO: 2 - protein, 965 aa, Artificial Sequence
  Truncated lactase fragment
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGAL

SEQ ID NO: 3 - protein, 1038 aa, Artificial Sequence
  Truncated lactase fragment
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQ

SEQ ID NO: 4 - protein, 1142 aa, Artificial Sequence
  Truncated lactase fragment
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQI

SEQ ID NO: 5 - protein, 1211 aa, Artificial Sequence
  Truncated lactase fragment
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTAT

SEQ ID NO: 6 - protein, 1296 aa, Artificial Sequence
  Truncated lactase fragment
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEF

SEQ ID NO: 7 - protein, 453 aa, Bifidobacterium bifidum
QNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTY

SEQ ID NO: 8 - DNA, 5160 bp, Artificial Sequence
  Nucleotide sequence encoding an extracellular lactase
gcagttgaagatgcaacaagaagcgatagcacaacacaaatgtcatcaacaccggaagttgtttattcatcagcggtcgatagcaaacaaaatcgcacaagcgattttgatgcgaactggaaatttatgctgtcagatagcgttcaagcacaagatccggcatttgatgattcagcatggcaacaagttgatctgccgcatgattatagcatcacacagaaatatagccaaagcaatgaagcagaatcagcatatcttccgggaggcacaggctggtatagaaaaagctttacaattgatagagatctggcaggcaaacgcattgcgattaattttgatggcgtctatatgaatgcaacagtctggtttaatggcgttaaactgggcacacatccgtatggctattcaccgttttcatttgatctgacaggcaatgcaaaatttggcggagaaaacacaattgtcgtcaaagttgaaaatagactgccgtcatcaagatggtattcaggcagcggcatttatagagatgttacactgacagttacagatggcgttcatgttggcaataatggcgtcgcaattaaaacaccgtcactggcaacacaaaatggcggagatgtcacaatgaacctgacaacaaaagtcgcgaatgatacagaagcagcagcgaacattacactgaaacagacagtttttccgaaaggcggaaaaacggatgcagcaattggcacagttacaacagcatcaaaatcaattgcagcaggcgcatcagcagatgttacaagcacaattacagcagcaagcccgaaactgtggtcaattaaaaacccgaacctgtatacagttagaacagaagttctgaacggaggcaaagttctggatacatatgatacagaatatggctttcgctggacaggctttgatgcaacatcaggcttttcactgaatggcgaaaaagtcaaactgaaaggcgttagcatgcatcatgatcaaggctcacttggcgcagttgcaaatagacgcgcaattgaaagacaagtcgaaatcctgcaaaaaatgggcgtcaatagcattcgcacaacacataatccggcagcaaaagcactgattgatgtctgcaatgaaaaaggcgttctggttgtcgaagaagtctttgatatgtggaaccgcagcaaaaatggcaacacggaagattatggcaaatggtttggccaagcaattgcaggcgataatgcagttctgggaggcgataaagatgaaacatgggcgaaatttgatcttacatcaacaattaaccgcgatagaaatgcaccgtcagttattatgtggtcactgggcaatgaaatgatggaaggcatttcaggctcagtttcaggctttccggcaacatcagcaaaactggttgcatggacaaaagcagcagattcaacaagaccgatgacatatggcgataacaaaattaaagcgaactggaacgaatcaaatacaatgggcgataatctgacagcaaatggcggagttgttggcacaaattattcagatggcgcaaactatgataaaattcgtacaacacatccgtcatgggcaatttatggctcagaaacagcatcagcgattaatagccgtggcatttataatagaacaacaggcggagcacaatcatcagataaacagctgacaagctatgataattcagcagttggctggggagcagttgcatcatcagcatggtatgatgttgttcagagagattttgtcgcaggcacatatgtttggacaggatttgattatctgggcgaaccgacaccgtggaatggcacaggctcaggcgcagttggctcatggccgtcaccgaaaaatagctattttggcatcgttgatacagcaggctttccgaaagatacatattatttttatcagagccagtggaatgatgatgttcatacactgcatattcttccggcatggaatgaaaatgttgttgcaaaaggctcaggcaataatgttccggttgtcgtttatacagatgcagcgaaagtgaaactgtattttacaccgaaaggctcaacagaaaaaagactgatcggcgaaaaatcatttacaaaaaaaacaacagcggcaggctatacatatcaagtctatgaaggcagcgataaagattcaacagcgcataaaaacatgtatctgacatggaatgttccgtgggcagaaggcacaatttcagcggaagcgtatgatgaaaataatcgcctgattccggaaggcagcacagaaggcaacgcatcagttacaacaacaggcaaagcagcaaaactgaaagcagatgcggatcgcaaaacaattacagcggatggcaaagatctgtcatatattgaagtcgatgtcacagatgcaaatggccatattgttccggatgcagcaaatagagtcacatttgatgttaaaggcgcaggcaaactggttggcgttgataatggctcatcaccggatcatgattcatatcaagcggataaccgcaaagcattttcaggcaaagtcctggcaattgttcagtcaacaaaagaagcaggcgaaattacagttacagcaaaagcagatggcctgcaatcaagcacagttaaaattgcaacaacagcagttccgggaacaagcacagaaaaaacagtccgcagcttttattacagccgcaactattatgtcaaaacaggcaacaaaccgattctgccgtcagatgttgaagttcgctattcagatggaacaagcgatagacaaaacgttacatgggatgcagtttcagatgatcaaattgcaaaagcaggctcattttcagttgcaggcacagttgcaggccaaaaaattagcgttcgcgtcacaatgattgatgaaattggcgcactgctgaattattcagcaagcacaccggttggcacaccggcagttcttccgggatcaagaccggcagtcctgccggatggcacagtcacatcagcaaattttgcagtccattggacaaaaccggcagatacagtctataatacagcaggcacagtcaaagtaccgggaacagcaacagtttttggcaaagaatttaaagtcacagcgacaattagagttcaaagaagccaagttacaattggctcatcagtttcaggaaatgcactgagactgacacaaaatattccggcagataaacaatcagatacactggatgcgattaaagatggctcaacaacagttgatgcaaatacaggcggaggcgcaaatccgtcagcatggacaaattgggcatattcaaaagcaggccataacacagcggaaattacatttgaatatgcgacagaacaacaactgggccagatcgtcatgtatttttttcgcgatagcaatgcagttagatttccggatgctggcaaaacaaaaattcagatcagcgcagatggcaaaaattggacagatctggcagcaacagaaacaattgcagcgcaagaatcaagcgatagagtcaaaccgtatacatatgattttgcaccggttggcgcaacatttgttaaagtgacagtcacaaacgcagatacaacaacaccgtcaggcgttgtttgcgcaggcctgacagaaattgaactgaaaacagcgacaagcaaatttgtcacaaatacatcagcagcactgtcatcacttacagtcaatggcacaaaagtttcagattcagttctggcagcaggctcatataacacaccggcaattatcgcagatgttaaagcggaaggcgaaggcaatgcaagcgttacagtccttccggcacatgataatgttattcgcgtcattacagaaagcgaagatcatgtcacacgcaaaacatttacaatcaacctgggcacagaacaagaatttccggctgattcagatgaaagagattatccggcagcagatatgacagtcacagttggctcagaacaaacatcaggcacagcaacagaaggaccgaaaaaatttgcagtcgatggcaacacatcaacatattggcatagcaattggacaccgacaacagttaatgatctgtggatcgcgtttgaactgcaaaaaccgacaaaactggatgcactgagatatcttccgcgtccggcaggctcaaaaaatggcagcgtcacagaatataaagttcaggtgtcagatgatggaacaaactggacagatgcaggctcaggcacatggacaacggattatggctggaaactggcggaatttaatcaaccggtcacaacaaaacatgttagactgaaagcggttcatacatatgcagatagcggcaacgataaatttatgagcgcaagcgaaattagactgagaaaagcggtcgatacaacggatatttcaggcgcaacagttacagttccggcaaaactgacagttgatagagttgatgcagatcatccggcaacatttgcaacaaaagatgtcacagttacactgggagatgcaacactgagatatggcgttgattatctgctggattatgcaggcaatacagcagttggcaaagcaacagtgacagttagaggcattgataaatattcaggcacagtcgcgaaaacatttacaattgaactgaaaaatgcaccggcaccggaaccgacactgacatcagttagcgtcaaaacaaaaccgagcaaactgacatatgttgtcggagatgcatttgatccggcaggcctggttctgcaacatgatagacaagcagatagacctccgcaaccgctggttggcgaacaagcggatgaacgcggactgacatgcggcacaagatgcgatagagttgaacaactgcgcaaacatgaaaatagagaagcgcatagaacaggcctggatcatctggaatttgttggcgcagcagatggcgcagttggagaacaagcaacatttaaagtccatgtccatgcagatcagggagatggcagacatgatgatgcagatgaacgcgatattgatccgcatgttccggtcgatcatgcagttggcgaactggcaagagcagcatgccatcatgttattggcctgagagtcgatacacatagacttaaagcaagcggctttcaaattccggctgatgatatggcagaaatcgatcgcattacaggctttcatcgttttgaacgccatgtc

SEQ ID NO: 9 - DNA, 2661 bp, Artificial Sequence
  Nucleotide sequence encoding BIF_917
gttgaagatgcaacaagaagcgatagcacaacacaaatgtcatcaacaccggaagttgtttattcatcagcggtcgatagcaaacaaaatcgcacaagcgattttgatgcgaactggaaatttatgctgtcagatagcgttcaagcacaagatccggcatttgatgattcagcatggcaacaagttgatctgccgcatgattatagcatcacacagaaatatagccaaagcaatgaagcagaatcagcatatcttccgggaggcacaggctggtatagaaaaagctttacaattgatagagatctggcaggcaaacgcattgcgattaattttgatggcgtctatatgaatgcaacagtctggtttaatggcgttaaactgggcacacatccgtatggctattcaccgttttcatttgatctgacaggcaatgcaaaatttggcggagaaaacacaattgtcgtcaaagttgaaaatagactgccgtcatcaagatggtattcaggcagcggcatttatagagatgttacactgacagttacagatggcgttcatgttggcaataatggcgtcgcaattaaaacaccgtcactggcaacacaaaatggcggagatgtcacaatgaacctgacaacaaaagtcgcgaatgatacagaagcagcagcgaacattacactgaaacagacagtttttccgaaaggcggaaaaacggatgcagcaattggcacagttacaacagcatcaaaatcaattgcagcaggcgcatcagcagatgttacaagcacaattacagcagcaagcccgaaactgtggtcaattaaaaacccgaacctgtatacagttagaacagaagttctgaacggaggcaaagttctggatacatatgatacagaatatggctttcgctggacaggctttgatgcaacatcaggcttttcactgaatggcgaaaaagtcaaactgaaaggcgttagcatgcatcatgatcaaggctcacttggcgcagttgcaaatagacgcgcaattgaaagacaagtcgaaatcctgcaaaaaatgggcgtcaatagcattcgcacaacacataatccggcagcaaaagcactgattgatgtctgcaatgaaaaaggcgttctggttgtcgaagaagtctttgatatgtggaaccgcagcaaaaatggcaacacggaagattatggcaaatggtttggccaagcaattgcaggcgataatgcagttctgggaggcgataaagatgaaacatgggcgaaatttgatcttacatcaacaattaaccgcgatagaaatgcaccgtcagttattatgtggtcactgggcaatgaaatgatggaaggcatttcaggctcagtttcaggctttccggcaacatcagcaaaactggttgcatggacaaaagcagcagattcaacaagaccgatgacatatggcgataacaaaattaaagcgaactggaacgaatcaaatacaatgggcgataatctgacagcaaatggcggagttgttggcacaaattattcagatggcgcaaactatgataaaattcgtacaacacatccgtcatgggcaatttatggctcagaaacagcatcagcgattaatagccgtggcatttataatagaacaacaggcggagcacaatcatcagataaacagctgacaagctatgataattcagcagttggctggggagcagttgcatcatcagcatggtatgatgttgttcagagagattttgtcgcaggcacatatgtttggacaggatttgattatctgggcgaaccgacaccgtggaatggcacaggctcaggcgcagttggctcatggccgtcaccgaaaaatagctattttggcatcgttgatacagcaggctttccgaaagatacatattatttttatcagagccagtggaatgatgatgttcatacactgcatattcttccggcatggaatgaaaatgttgttgcaaaaggctcaggcaataatgttccggttgtcgtttatacagatgcagcgaaagtgaaactgtattttacaccgaaaggctcaacagaaaaaagactgatcggcgaaaaatcatttacaaaaaaaacaacagcggcaggctatacatatcaagtctatgaaggcagcgataaagattcaacagcgcataaaaacatgtatctgacatggaatgttccgtgggcagaaggcacaatttcagcggaagcgtatgatgaaaataatcgcctgattccggaaggcagcacagaaggcaacgcatcagttacaacaacaggcaaagcagcaaaactgaaagcagatgcggatcgcaaaacaattacagcggatggcaaagatctgtcatatattgaagtcgatgtcacagatgcaaatggccatattgttccggatgcagcaaatagagtcacatttgatgttaaaggcgcaggcaaactggttggcgttgataatggctcatcaccggatcatgattcatatcaagcggataaccgcaaagcattttcaggcaaagtcctggcaattgttcagtcaacaaaagaagcaggcgaaattacagttacagcaaaagcagatggcctgcaatcaagcacagttaaaattgcaacaacagcagttccgggaacaagcacagaaaaaaca

SEQ ID NO: 10 - DNA, 2895 bp, Artificial Sequence
  Nucleotide sequence encoding BIF_995
gttgaagatgcaacaagaagcgatagcacaacacaaatgtcatcaacaccggaagttgtttattcatcagcggtcgatagcaaacaaaatcgcacaagcgattttgatgcgaactggaaatttatgctgtcagatagcgttcaagcacaagatccggcatttgatgattcagcatggcaacaagttgatctgccgcatgattatagcatcacacagaaatatagccaaagcaatgaagcagaatcagcatatcttccgggaggcacaggctggtatagaaaaagctttacaattgatagagatctggcaggcaaacgcattgcgattaattttgatggcgtctatatgaatgcaacagtctggtttaatggcgttaaactgggcacacatccgtatggctattcaccgttttcatttgatctgacaggcaatgcaaaatttggcggagaaaacacaattgtcgtcaaagttgaaaatagactgccgtcatcaagatggtattcaggcagcggcatttatagagatgttacactgacagttacagatggcgttcatgttggcaataatggcgtcgcaattaaaacaccgtcactggcaacacaaaatggcggagatgtcacaatgaacctgacaacaaaagtcgcgaatgatacagaagcagcagcgaacattacactgaaacagacagtttttccgaaaggcggaaaaacggatgcagcaattggcacagttacaacagcatcaaaatcaattgcagcaggcgcatcagcagatgttacaagcacaattacagcagcaagcccgaaactgtggtcaattaaaaacccgaacctgtatacagttagaacagaagttctgaacggaggcaaagttctggatacatatgatacagaatatggctttcgctggacaggctttgatgcaacatcaggcttttcactgaatggcgaaaaagtcaaactgaaaggcgttagcatgcatcatgatcaaggctcacttggcgcagttgcaaatagacgcgcaattgaaagacaagtcgaaatcctgcaaaaaatgggcgtcaatagcattcgcacaacacataatccggcagcaaaagcactgattgatgtctgcaatgaaaaaggcgttctggttgtcgaagaagtctttgatatgtggaaccgcagcaaaaatggcaacacggaagattatggcaaatggtttggccaagcaattgcaggcgataatgcagttctgggaggcgataaagatgaaacatgggcgaaatttgatcttacatcaacaattaaccgcgatagaaatgcaccgtcagttattatgtggtcactgggcaatgaaatgatggaaggcatttcaggctcagtttcaggctttccggcaacatcagcaaaactggttgcatggacaaaagcagcagattcaacaagaccgatgacatatggcgataacaaaattaaagcgaactggaacgaatcaaatacaatgggcgataatctgacagcaaatggcggagttgttggcacaaattattcagatggcgcaaactatgataaaattcgtacaacacatccgtcatgggcaatttatggctcagaaacagcatcagcgattaatagccgtggcatttataatagaacaacaggcggagcacaatcatcagataaacagctgacaagctatgataattcagcagttggctggggagcagttgcatcatcagcatggtatgatgttgttcagagagattttgtcgcaggcacatatgtttggacaggatttgattatctgggcgaaccgacaccgtggaatggcacaggctcaggcgcagttggctcatggccgtcaccgaaaaatagctattttggcatcgttgatacagcaggctttccgaaagatacatattatttttatcagagccagtggaatgatgatgttcatacactgcatattcttccggcatggaatgaaaatgttgttgcaaaaggctcaggcaataatgttccggttgtcgtttatacagatgcagcgaaagtgaaactgtattttacaccgaaaggctcaacagaaaaaagactgatcggcgaaaaatcatttacaaaaaaaacaacagcggcaggctatacatatcaagtctatgaaggcagcgataaagattcaacagcgcataaaaacatgtatctgacatggaatgttccgtgggcagaaggcacaatttcagcggaagcgtatgatgaaaataatcgcctgattccggaaggcagcacagaaggcaacgcatcagttacaacaacaggcaaagcagcaaaactgaaagcagatgcggatcgcaaaacaattacagcggatggcaaagatctgtcatatattgaagtcgatgtcacagatgcaaatggccatattgttccggatgcagcaaatagagtcacatttgatgttaaaggcgcaggcaaactggttggcgttgataatggctcatcaccggatcatgattcatatcaagcggataaccgcaaagcattttcaggcaaagtcctggcaattgttcagtcaacaaaagaagcaggcgaaattacagttacagcaaaagcagatggcctgcaatcaagcacagttaaaattgcaacaacagcagttccgggaacaagcacagaaaaaacagtccgcagcttttattacagccgcaactattatgtcaaaacaggcaacaaaccgattctgccgtcagatgttgaagttcgctattcagatggaacaagcgatagacaaaacgttacatgggatgcagtttcagatgatcaaattgcaaaagcaggctcattttcagttgcaggcacagttgcaggccaaaaaattagcgttcgcgtcacaatgattgatgaaattggcgcactg

SEQ ID NO: 11 - DNA, 3114 bp, Artificial Sequence
  Nucleotide sequence encoding BIF_1068
gttgaagatgcaacaagaagcgatagcacaacacaaatgtcatcaacaccggaagttgtttattcatcagcggtcgatagcaaacaaaatcgcacaagcgattttgatgcgaactggaaatttatgctgtcagatagcgttcaagcacaagatccggcatttgatgattcagcatggcaacaagttgatctgccgcatgattatagcatcacacagaaatatagccaaagcaatgaagcagaatcagcatatcttccgggaggcacaggctggtatagaaaaagctttacaattgatagagatctggcaggcaaacgcattgcgattaattttgatggcgtctatatgaatgcaacagtctggtttaatggcgttaaactgggcacacatccgtatggctattcaccgttttcatttgatctgacaggcaatgcaaaatttggcggagaaaacacaattgtcgtcaaagttgaaaatagactgccgtcatcaagatggtattcaggcagcggcatttatagagatgttacactgacagttacagatggcgttcatgttggcaataatggcgtcgcaattaaaacaccgtcactggcaacacaaaatggcggagatgtcacaatgaacctgacaacaaaagtcgcgaatgatacagaagcagcagcgaacattacactgaaacagacagtttttccgaaaggcggaaaaacggatgcagcaattggcacagttacaacagcatcaaaatcaattgcagcaggcgcatcagcagatgttacaagcacaattacagcagcaagcccgaaactgtggtcaattaaaaacccgaacctgtatacagttagaacagaagttctgaacggaggcaaagttctggatacatatgatacagaatatggctttcgctggacaggctttgatgcaacatcaggcttttcactgaatggcgaaaaagtcaaactgaaaggcgttagcatgcatcatgatcaaggctcacttggcgcagttgcaaatagacgcgcaattgaaagacaagtcgaaatcctgcaaaaaatgggcgtcaatagcattcgcacaacacataatccggcagcaaaagcactgattgatgtctgcaatgaaaaaggcgttctggttgtcgaagaagtctttgatatgtggaaccgcagcaaaaatggcaacacggaagattatggcaaatggtttggccaagcaattgcaggcgataatgcagttctgggaggcgataaagatgaaacatgggcgaaatttgatcttacatcaacaattaaccgcgatagaaatgcaccgtcagttattatgtggtcactgggcaatgaaatgatggaaggcatttcaggctcagtttcaggctttccggcaacatcagcaaaactggttgcatggacaaaagcagcagattcaacaagaccgatgacatatggcgataacaaaattaaagcgaactggaacgaatcaaatacaatgggcgataatctgacagcaaatggcggagttgttggcacaaattattcagatggcgcaaactatgataaaattcgtacaacacatccgtcatgggcaatttatggctcagaaacagcatcagcgattaatagccgtggcatttataatagaacaacaggcggagcacaatcatcagataaacagctgacaagctatgataattcagcagttggctggggagcagttgcatcatcagcatggtatgatgttgttcagagagattttgtcgcaggcacatatgtttggacaggatttgattatctgggcgaaccgacaccgtggaatggcacaggctcaggcgcagttggctcatggccgtcaccgaaaaatagctattttggcatcgttgatacagcaggctttccgaaagatacatattatttttatcagagccagtggaatgatgatgttcatacactgcatattcttccggcatggaatgaaaatgttgttgcaaaaggctcaggcaataatgttccggttgtcgtttatacagatgcagcgaaagtgaaactgtattttacaccgaaaggctcaacagaaaaaagactgatcggcgaaaaatcatttacaaaaaaaacaacagcggcaggctatacatatcaagtctatgaaggcagcgataaagattcaacagcgcataaaaacatgtatctgacatggaatgttccgtgggcagaaggcacaatttcagcggaagcgtatgatgaaaataatcgcctgattccggaaggcagcacagaaggcaacgcatcagttacaacaacaggcaaagcagcaaaactgaaagcagatgcggatcgcaaaacaattacagcggatggcaaagatctgtcatatattgaagtcgatgtcacagatgcaaatggccatattgttccggatgcagcaaatagagtcacatttgatgttaaaggcgcaggcaaactggttggcgttgataatggctcatcaccggatcatgattcatatcaagcggataaccgcaaagcattttcaggcaaagtcctggcaattgttcagtcaacaaaagaagcaggcgaaattacagttacagcaaaagcagatggcctgcaatcaagcacagttaaaattgcaacaacagcagttccgggaacaagcacagaaaaaacagtccgcagcttttattacagccgcaactattatgtcaaaacaggcaacaaaccgattctgccgtcagatgttgaagttcgctattcagatggaacaagcgatagacaaaacgttacatgggatgcagtttcagatgatcaaattgcaaaagcaggctcattttcagttgcaggcacagttgcaggccaaaaaattagcgttcgcgtcacaatgattgatgaaattggcgcactgctgaattattcagcaagcacaccggttggcacaccggcagttcttccgggatcaagaccggcagtcctgccggatggcacagtcacatcagcaaattttgcagtccattggacaaaaccggcagatacagtctataatacagcaggcacagtcaaagtaccgggaacagcaacagtttttggcaaagaatttaaagtcacagcgacaattagagttcaa

SEQ ID NO: 12 - DNA, 3426 bp, Artificial Sequence
  Nucleotide sequence encoding BIF_1172
gttgaagatgcaacaagaagcgatagcacaacacaaatgtcatcaacaccggaagttgtttattcatcagcggtcgatagcaaacaaaatcgcacaagcgattttgatgcgaactggaaatttatgctgtcagatagcgttcaagcacaagatccggcatttgatgattcagcatggcaacaagttgatctgccgcatgattatagcatcacacagaaatatagccaaagcaatgaagcagaatcagcatatcttccgggaggcacaggctggtatagaaaaagctttacaattgatagagatctggcaggcaaacgcattgcgattaattttgatggcgtctatatgaatgcaacagtctggtttaatggcgttaaactgggcacacatccgtatggctattcaccgttttcatttgatctgacaggcaatgcaaaatttggcggagaaaacacaattgtcgtcaaagttgaaaatagactgccgtcatcaagatggtattcaggcagcggcatttatagagatgttacactgacagttacagatggcgttcatgttggcaataatggcgtcgcaattaaaacaccgtcactggcaacacaaaatggcggagatgtcacaatgaacctgacaacaaaagtcgcgaatgatacagaagcagcagcgaacattacactgaaacagacagtttttccgaaaggcggaaaaacggatgcagcaattggcacagttacaacagcatcaaaatcaattgcagcaggcgcatcagcagatgttacaagcacaattacagcagcaagcccgaaactgtggtcaattaaaaacccgaacctgtatacagttagaacagaagttctgaacggaggcaaagttctggatacatatgatacagaatatggctttcgctggacaggctttgatgcaacatcaggcttttcactgaatggcgaaaaagtcaaactgaaaggcgttagcatgcatcatgatcaaggctcacttggcgcagttgcaaatagacgcgcaattgaaagacaagtcgaaatcctgcaaaaaatgggcgtcaatagcattcgcacaacacataatccggcagcaaaagcactgattgatgtctgcaatgaaaaaggcgttctggttgtcgaagaagtctttgatatgtggaaccgcagcaaaaatggcaacacggaagattatggcaaatggtttggccaagcaattgcaggcgataatgcagttctgggaggcgataaagatgaaacatgggcgaaatttgatcttacatcaacaattaaccgcgatagaaatgcaccgtcagttattatgtggtcactgggcaatgaaatgatggaaggcatttcaggctcagtttcaggctttccggcaacatcagcaaaactggttgcatggacaaaagcagcagattcaacaagaccgatgacatatggcgataacaaaattaaagcgaactggaacgaatcaaatacaatgggcgataatctgacagcaaatggcggagttgttggcacaaattattcagatggcgcaaactatgataaaattcgtacaacacatccgtcatgggcaatttatggctcagaaacagcatcagcgattaatagccgtggcatttataatagaacaacaggcggagcacaatcatcagataaacagctgacaagctatgataattcagcagttggctggggagcagttgcatcatcagcatggtatgatgttgttcagagagattttgtcgcaggcacatatgtttggacaggatttgattatctgggcgaaccgacaccgtggaatggcacaggctcaggcgcagttggctcatggccgtcaccgaaaaatagctattttggcatcgttgatacagcaggctttccgaaagatacatattatttttatcagagccagtggaatgatgatgttcatacactgcatattcttccggcatggaatgaaaatgttgttgcaaaaggctcaggcaataatgttccggttgtcgtttatacagatgcagcgaaagtgaaactgtattttacaccgaaaggctcaacagaaaaaagactgatcggcgaaaaatcatttacaaaaaaaacaacagcggcaggctatacatatcaagtctatgaaggcagcgataaagattcaacagcgcataaaaacatgtatctgacatggaatgttccgtgggcagaaggcacaatttcagcggaagcgtatgatgaaaataatcgcctgattccggaaggcagcacagaaggcaacgcatcagttacaacaacaggcaaagcagcaaaactgaaagcagatgcggatcgcaaaacaattacagcggatggcaaagatctgtcatatattgaagtcgatgtcacagatgcaaatggccatattgttccggatgcagcaaatagagtcacatttgatgttaaaggcgcaggcaaactggttggcgttgataatggctcatcaccggatcatgattcatatcaagcggataaccgcaaagcattttcaggcaaagtcctggcaattgttcagtcaacaaaagaagcaggcgaaattacagttacagcaaaagcagatggcctgcaatcaagcacagttaaaattgcaacaacagcagttccgggaacaagcacagaaaaaacagtccgcagcttttattacagccgcaactattatgtcaaaacaggcaacaaaccgattctgccgtcagatgttgaagttcgctattcagatggaacaagcgatagacaaaacgttacatgggatgcagtttcagatgatcaaattgcaaaagcaggctcattttcagttgcaggcacagttgcaggccaaaaaattagcgttcgcgtcacaatgattgatgaaattggcgcactgctgaattattcagcaagcacaccggttggcacaccggcagttcttccgggatcaagaccggcagtcctgccggatggcacagtcacatcagcaaattttgcagtccattggacaaaaccggcagatacagtctataatacagcaggcacagtcaaagtaccgggaacagcaacagtttttggcaaagaatttaaagtcacagcgacaattagagttcaaagaagccaagttacaattggctcatcagtttcaggaaatgcactgagactgacacaaaatattccggcagataaacaatcagatacactggatgcgattaaagatggctcaacaacagttgatgcaaatacaggcggaggcgcaaatccgtcagcatggacaaattgggcatattcaaaagcaggccataacacagcggaaattacatttgaatatgcgacagaacaacaactgggccagatcgtcatgtatttttttcgcgatagcaatgcagttagatttccggatgctggcaaaacaaaaattcagatc

SEQ ID NO: 13 - DNA, 3633 bp, Artificial Sequence
  Nucleotide sequence encoding BIF_1241
gttgaagatgcaacaagaagcgatagcacaacacaaatgtcatcaacaccggaagttgtttattcatcagcggtcgatagcaaacaaaatcgcacaagcgattttgatgcgaactggaaatttatgctgtcagatagcgttcaagcacaagatccggcatttgatgattcagcatggcaacaagttgatctgccgcatgattatagcatcacacagaaatatagccaaagcaatgaagcagaatcagcatatcttccgggaggcacaggctggtatagaaaaagctttacaattgatagagatctggcaggcaaacgcattgcgattaattttgatggcgtctatatgaatgcaacagtctggtttaatggcgttaaactgggcacacatccgtatggctattcaccgttttcatttgatctgacaggcaatgcaaaatttggcggagaaaacacaattgtcgtcaaagttgaaaatagactgccgtcatcaagatggtattcaggcagcggcatttatagagatgttacactgacagttacagatggcgttcatgttggcaataatggcgtcgcaattaaaacaccgtcactggcaacacaaaatggcggagatgtcacaatgaacctgacaacaaaagtcgcgaatgatacagaagcagcagcgaacattacactgaaacagacagtttttccgaaaggcggaaaaacggatgcagcaattggcacagttacaacagcatcaaaatcaattgcagcaggcgcatcagcagatgttacaagcacaattacagcagcaagcccgaaactgtggtcaattaaaaacccgaacctgtatacagttagaacagaagttctgaacggaggcaaagttctggatacatatgatacagaatatggctttcgctggacaggctttgatgcaacatcaggcttttcactgaatggcgaaaaagtcaaactgaaaggcgttagcatgcatcatgatcaaggctcacttggcgcagttgcaaatagacgcgcaattgaaagacaagtcgaaatcctgcaaaaaatgggcgtcaatagcattcgcacaacacataatccggcagcaaaagcactgattgatgtctgcaatgaaaaaggcgttctggttgtcgaagaagtctttgatatgtggaaccgcagcaaaaatggcaacacggaagattatggcaaatggtttggccaagcaattgcaggcgataatgcagttctgggaggcgataaagatgaaacatgggcgaaatttgatcttacatcaacaattaaccgcgatagaaatgcaccgtcagttattatgtggtcactgggcaatgaaatgatggaaggcatttcaggctcagtttcaggctttccggcaacatcagcaaaactggttgcatggacaaaagcagcagattcaacaagaccgatgacatatggcgataacaaaattaaagcgaactggaacgaatcaaatacaatgggcgataatctgacagcaaatggcggagttgttggcacaaattattcagatggcgcaaactatgataaaattcgtacaacacatccgtcatgggcaatttatggctcagaaacagcatcagcgattaatagccgtggcatttataatagaacaacaggcggagcacaatcatcagataaacagctgacaagctatgataattcagcagttggctggggagcagttgcatcatcagcatggtatgatgttgttcagagagattttgtcgcaggcacatatgtttggacaggatttgattatctgggcgaaccgacaccgtggaatggcacaggctcaggcgcagttggctcatggccgtcaccgaaaaatagctattttggcatcgttgatacagcaggctttccgaaagatacatattatttttatcagagccagtggaatgatgatgttcatacactgcatattcttccggcatggaatgaaaatgttgttgcaaaaggctcaggcaataatgttccggttgtcgtttatacagatgcagcgaaagtgaaactgtattttacaccgaaaggctcaacagaaaaaagactgatcggcgaaaaatcatttacaaaaaaaacaacagcggcaggctatacatatcaagtctatgaaggcagcgataaagattcaacagcgcataaaaacatgtatctgacatggaatgttccgtgggcagaaggcacaatttcagcggaagcgtatgatgaaaataatcgcctgattccggaaggcagcacagaaggcaacgcatcagttacaacaacaggcaaagcagcaaaactgaaagcagatgcggatcgcaaaacaattacagcggatggcaaagatctgtcatatattgaagtcgatgtcacagatgcaaatggccatattgttccggatgcagcaaatagagtcacatttgatgttaaaggcgcaggcaaactggttggcgttgataatggctcatcaccggatcatgattcatatcaagcggataaccgcaaagcattttcaggcaaagtcctggcaattgttcagtcaacaaaagaagcaggcgaaattacagttacagcaaaagcagatggcctgcaatcaagcacagttaaaattgcaacaacagcagttccgggaacaagcacagaaaaaacagtccgcagcttttattacagccgcaactattatgtcaaaacaggcaacaaaccgattctgccgtcagatgttgaagttcgctattcagatggaacaagcgatagacaaaacgttacatgggatgcagtttcagatgatcaaattgcaaaagcaggctcattttcagttgcaggcacagttgcaggccaaaaaattagcgttcgcgtcacaatgattgatgaaattggcgcactgctgaattattcagcaagcacaccggttggcacaccggcagttcttccgggatcaagaccggcagtcctgccggatggcacagtcacatcagcaaattttgcagtccattggacaaaaccggcagatacagtctataatacagcaggcacagtcaaagtaccgggaacagcaacagtttttggcaaagaatttaaagtcacagcgacaattagagttcaaagaagccaagttacaattggctcatcagtttcaggaaatgcactgagactgacacaaaatattccggcagataaacaatcagatacactggatgcgattaaagatggctcaacaacagttgatgcaaatacaggcggaggcgcaaatccgtcagcatggacaaattgggcatattcaaaagcaggccataacacagcggaaattacatttgaatatgcgacagaacaacaactgggccagatcgtcatgtatttttttcgcgatagcaatgcagttagatttccggatgctggcaaaacaaaaattcagatcagcgcagatggcaaaaattggacagatctggcagcaacagaaacaattgcagcgcaagaatcaagcgatagagtcaaaccgtatacatatgattttgcaccggttggcgcaacatttgttaaagtgacagtcacaaacgcagatacaacaacaccgtcaggcgttgtttgcgcaggcctgacagaaattgaactgaaaacagcgaca

SEQ ID NO: 14 - DNA, 3888 bp, Artificial Sequence
  Nucleotide sequence encoding BIF_1326
gttgaagatgcaacaagaagcgatagcacaacacaaatgtcatcaacaccggaagttgtttattcatcagcggtcgatagcaaacaaaatcgcacaagcgattttgatgcgaactggaaatttatgctgtcagatagcgttcaagcacaagatccggcatttgatgattcagcatggcaacaagttgatctgccgcatgattatagcatcacacagaaatatagccaaagcaatgaagcagaatcagcatatcttccgggaggcacaggctggtatagaaaaagctttacaattgatagagatctggcaggcaaacgcattgcgattaattttgatggcgtctatatgaatgcaacagtctggtttaatggcgttaaactgggcacacatccgtatggctattcaccgttttcatttgatctgacaggcaatgcaaaatttggcggagaaaacacaattgtcgtcaaagttgaaaatagactgccgtcatcaagatggtattcaggcagcggcatttatagagatgttacactgacagttacagatggcgttcatgttggcaataatggcgtcgcaattaaaacaccgtcactggcaacacaaaatggcggagatgtcacaatgaacctgacaacaaaagtcgcgaatgatacagaagcagcagcgaacattacactgaaacagacagtttttccgaaaggcggaaaaacggatgcagcaattggcacagttacaacagcatcaaaatcaattgcagcaggcgcatcagcagatgttacaagcacaattacagcagcaagcccgaaactgtggtcaattaaaaacccgaacctgtatacagttagaacagaagttctgaacggaggcaaagttctggatacatatgatacagaatatggctttcgctggacaggctttgatgcaacatcaggcttttcactgaatggcgaaaaagtcaaactgaaaggcgttagcatgcatcatgatcaaggctcacttggcgcagttgcaaatagacgcgcaattgaaagacaagtcgaaatcctgcaaaaaatgggcgtcaatagcattcgcacaacacataatccggcagcaaaagcactgattgatgtctgcaatgaaaaaggcgttctggttgtcgaagaagtctttgatatgtggaaccgcagcaaaaatggcaacacggaagattatggcaaatggtttggccaagcaattgcaggcgataatgcagttctgggaggcgataaagatgaaacatgggcgaaatttgatcttacatcaacaattaaccgcgatagaaatgcaccgtcagttattatgtggtcactgggcaatgaaatgatggaaggcatttcaggctcagtttcaggctttccggcaacatcagcaaaactggttgcatggacaaaagcagcagattcaacaagaccgatgacatatggcgataacaaaattaaagcgaactggaacgaatcaaatacaatgggcgataatctgacagcaaatggcggagttgttggcacaaattattcagatggcgcaaactatgataaaattcgtacaacacatccgtcatgggcaatttatggctcagaaacagcatcagcgattaatagccgtggcatttataatagaacaacaggcggagcacaatcatcagataaacagctgacaagctatgataattcagcagttggctggggagcagttgcatcatcagcatggtatgatgttgttcagagagattttgtcgcaggcacatatgtttggacaggatttgattatctgggcgaaccgacaccgtggaatggcacaggctcaggcgcagttggctcatggccgtcaccgaaaaatagctattttggcatcgttgatacagcaggctttccgaaagatacatattatttttatcagagccagtggaatgatgatgttcatacactgcatattcttccggcatggaatgaaaatgttgttgcaaaaggctcaggcaataatgttccggttgtcgtttatacagatgcagcgaaagtgaaactgtattttacaccgaaaggctcaacagaaaaaagactgatcggcgaaaaatcatttacaaaaaaaacaacagcggcaggctatacatatcaagtctatgaaggcagcgataaagattcaacagcgcataaaaacatgtatctgacatggaatgttccgtgggcagaaggcacaatttcagcggaagcgtatgatgaaaataatcgcctgattccggaaggcagcacagaaggcaacgcatcagttacaacaacaggcaaagcagcaaaactgaaagcagatgcggatcgcaaaacaattacagcggatggcaaagatctgtcatatattgaagtcgatgtcacagatgcaaatggccatattgttccggatgcagcaaatagagtcacatttgatgttaaaggcgcaggcaaactggttggcgttgataatggctcatcaccggatcatgattcatatcaagcggataaccgcaaagcattttcaggcaaagtcctggcaattgttcagtcaacaaaagaagcaggcgaaattacagttacagcaaaagcagatggcctgcaatcaagcacagttaaaattgcaacaacagcagttccgggaacaagcacagaaaaaacagtccgcagcttttattacagccgcaactattatgtcaaaacaggcaacaaaccgattctgccgtcagatgttgaagttcgctattcagatggaacaagcgatagacaaaacgttacatgggatgcagtttcagatgatcaaattgcaaaagcaggctcattttcagttgcaggcacagttgcaggccaaaaaattagcgttcgcgtcacaatgattgatgaaattggcgcactgctgaattattcagcaagcacaccggttggcacaccggcagttcttccgggatcaagaccggcagtcctgccggatggcacagtcacatcagcaaattttgcagtccattggacaaaaccggcagatacagtctataatacagcaggcacagtcaaagtaccgggaacagcaacagtttttggcaaagaatttaaagtcacagcgacaattagagttcaaagaagccaagttacaattggctcatcagtttcaggaaatgcactgagactgacacaaaatattccggcagataaacaatcagatacactggatgcgattaaagatggctcaacaacagttgatgcaaatacaggcggaggcgcaaatccgtcagcatggacaaattgggcatattcaaaagcaggccataacacagcggaaattacatttgaatatgcgacagaacaacaactgggccagatcgtcatgtatttttttcgcgatagcaatgcagttagatttccggatgctggcaaaacaaaaattcagatcagcgcagatggcaaaaattggacagatctggcagcaacagaaacaattgcagcgcaagaatcaagcgatagagtcaaaccgtatacatatgattttgcaccggttggcgcaacatttgttaaagtgacagtcacaaacgcagatacaacaacaccgtcaggcgttgtttgcgcaggcctgacagaaattgaactgaaaacagcgacaagcaaatttgtcacaaatacatcagcagcactgtcatcacttacagtcaatggcacaaaagtttcagattcagttctggcagcaggctcatataacacaccggcaattatcgcagatgttaaagcggaaggcgaaggcaatgcaagcgttacagtccttccggcacatgataatgttattcgcgtcattacagaaagcgaagatcatgtcacacgcaaaacatttacaatcaacctgggcacagaacaagaattt

SEQ ID NO: 15 - DNA, 30 bp, Artificial Sequence
  Forward primer for generation of BIF variants
ggggtaactagtggaagatgcaacaagaag

SEQ ID NO: 16 - DNA, 35 bp, Artificial Sequence
  Reverse primer for BIF917
gcgcttaattaattatgttttttctgtgcttgttc

SEQ ID NO: 17 - DNA, 38 bp, Artificial Sequence
  Reverse primer for BIF995
gcgcttaattaattacagtgcgccaatttcatcaatca

SEQ ID NO: 18 - DNA, 35 bp, Artificial Sequence
  Reverse primer for BIF1068
gcgcttaattaattattgaactctaattgtcgctg

SEQ ID NO: 19 - DNA, 36 bp, Artificial Sequence
  Reverse primer for BIF1241
gcgcttaattaattatgtcgctgttttcagttcaat

SEQ ID NO: 20 - DNA, 35 bp, Artificial Sequence
  Reverse primer for BIF1326
gcgcttaattaattaaaattcttgttctgtgccca

SEQ ID NO: 21 - DNA, 38 bp, Artificial Sequence
  Reverse primer for BIF1478
gcgcttaattaattatctcagtctaatttcgcttgcgc

SEQ ID NO: 22 - protein, 1720 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQHDRQADRPPQPLVGEQADERGLTCGTRCDRVEQLRKHENREAHRTGLDHLEFVGAADGAVGEQATFKVHVHADQGDGRHDDADERDIDPHVPVDHAVGELARAACHHVIGLRVDTHRLKASGFQIPADDMAEIDRITGFHRFERHVG

SEQ ID NO: 23 - protein, 30 aa, Bifidobacterium bifidum
VRSKKLWISLLFALALIFTMAFGSTSSAQA

(19) United States
(12) Patent Application Publication
(10) Pub. No.: US 2020/0296980 A1
(43) Pub. Date: Sep. 24, 2020

(54) METHOD OF GENERATING A SACCHARIDE CONTAINING A GALACTOSE AND A FRUCTOSE MOIETY EMPLOYING ENZYME WITH TRANSGALACTOSYLATING ACTIVITY

(71) Applicant: DUPONT NUTRITION BIOSCIENCES APS, Copenhagen (DK)

(72) Inventors: Thomas EISELE, Eichenau (DE); Jacob Flyvholm CRAMER, Palo Alto, CA (US); Morten Krog LARSEN, Sabro (DK)

(21) Appl. No.: 16/881,569

(22) Filed: May 22, 2020

Related U.S. Application Data
(63) Continuation of application No. 15/524,608, filed on May 4, 2017, now abandoned.

(51) Int. Cl. A23C 9/12; C07H 1/00; A23L 33/21; A23C 9/20; C12P 19/04; C12N 9/38; C07H 3/04; C13K 13/00; C07H 3/06; C12P 19/18; C12P 19/12

(52) U.S. Cl. CPC A23C 9/1206; C07H 1/00; A23L 33/21; C12Y 302/01023; A23C 9/203; C12P 19/04; C13K 13/00; C07H 3/04; C13K 13/005; C07H 3/06; C12P 19/18; C12P 19/12; C12N 9/2471

(57) ABSTRACT

A method of generating a saccharide, especially lactulose or lactosucrose, in the presence of a transgalactosylase enzyme, is described herein.

### FIELD OF THE INVENTION

This invention relates to a method of generating a saccharide, especially but not exclusively lactulose or lactosucrose, using an enzyme.

### BACKGROUND TO THE INVENTION

Lactulose, 4-O-β-D-galactopyranosyl-β-D-fructofuranose, is a disaccharide formed from one molecule each of the monosaccharides fructose and galactose. It is used as a laxative as it is not absorbed in the human intestine nor broken down by human enzymes, and thus stays in the digestive bolus through most of its course, causing retention of water through osmosis leading to softer, easier to pass stool. It has a secondary laxative effect in the colon, where it is fermented by the gut flora.

Lactulose is commonly used as a food additive to improve taste, promote intestinal health and promote intestinal transit time. Lactulose is known for its good acceptance, with limited side effects, similar to many other food products. It is used in the treatment of chronic constipation and hepatic encephalopathy, a complication of liver disease.

As described in Mayer et al. J. Agric. Food Chem. 2004, 52, 6983-6990, as a result of worldwide cheese production lactose accumulates in whey in amounts of millions of tons per year, and the residual amount causes increasing environmental problems with its disposal. Therefore, it is of commercial interest to develop alternative methods of exploitation of lactose.

Methods of synthesising lactulose from lactose, both chemical and enzymatic, are known in the art. For example, the above Mayer et al. article describes the bioconversion of lactose in the presence of fructose using the enzymes CeIB (an α-glycosidase from Pyrococcus furiosus) and β-galactosidase from Aspergillus oryzae. The process was carried out at lactose concentrations of 17, 34, 68, 136, 194 and 272 g/L and fructose concentrations of 18, 90, 144, 180, 270 and 360 g/L.

However, the yield of lactulose achieved by the process of Mayer et al. are low—the maximum concentration of lactulose is 3.4 g/L, equating to a yield of 2.7% by weight calculated on the total amount of applied sugar. i.e. the total weight of lactose and fructose starting material. In particular, the yields achieved at the lowest fructose concentrations are very low: this is because hydrolysis of lactose predominates at these fructose concentrations. In addition, the CeIB enzyme is thermostable with a temperature optimum of 105° C., making it difficult to completely inactivate in an industrial process.

Moreover, the synthetic processes described in the above Mayer et al. article are carried out at pH 5.0. A process carried out at this pH would be unsuitable for carrying out in situ in a milk composition, as this pH is below 5.5 which is the typical level at which the caseins in the milk would begin to form a gel, thereby increasing the viscosity of the milk composition.

Lee et al., Appl. Microbial Biotechnol. 2004, 64, 787-793, describes the enzymatic production of lactulose from lactose and fructose by permeabilized Kluyveromyces lactis cells in which β-galactosidase is generated. The process used lactose 30% w/v to 40% w/v (corresponding to 300 to 400 g/L) and fructose in a concentration varying from 10% w/v to 20% w/v (corresponding to 100 g/L to 200 g/L). The yields achieved were also low—a process using 40% w/v lactose and 20% w/v fructose produced 20 g/L lactulose, equating to a 3.33% lactulose yield by weight calculated on the total amount of applied sugar.

Kim et al., Enzyme and Microbial Technology, 2006, 39, 903-908, describes the enzymatic production of lactulose from lactose and fructose by a thermostable β-galactosidase from Sulfolobus solfataricus. The process used a minimum of 15% w/v lactose (corresponding to 150 g/L) and a minimum of 15% w/v fructose (corresponding to 150 g/L). The yields achieved were also low—this process produced 50 g/L lactulose, equating to an 8.33% lactulose yield by weight calculated on the total amount of applied sugar.

Vaheri and Kauppinen, Acta Pharmaceutica Fennica, 1978, 87, 75-83, also describes the enzymatic production of lactulose from lactose and fructose using various different β-galactosidases. Yields achieved according to these methods were also poor—starting from 12% (w/v) lactose and 20% (w/v) fructose, the maximum lactulose concentration achieved was 9 g/L, equating to a yield of 2.8% calculated on the total amount of applied sugar.

Förster-Fromme et al. International Dairy Journal, 2011, 21, 940-948, also describes a method for producing lactulose from lactose and fructose using a β-galactosidase enzyme. The process described in this document uses 90 g/L fructose. However, this was a pilot study aimed at testing bifidogenic effects of the lactulose: the high concentration of fructose would make a commercial product unacceptably too sweet.

Guerrero et al. J. Molec. Catal. B: Enzymatic, 2011, 72, 206-212, describes the enzymatic production of lactulose from lactose and fructose using three different commercial β-galactosidases. However, the majority of the processes described in this document are carried out at pH 4.5. For similar reasons to those set out above in relation to Mayer et al., a process carried out at this pH would be unsuitable for carrying out in situ in a milk composition. The processes described in this document which are carried out at a pH above 5.5 used 50% w/w total sugars in a molar ratio of lactose:fructose of 1:1 (corresponding to 0.957 mol/L of both lactose and fructose).

Adamczak et al. Chem Pap. 2009, 63, 111-116, describes the enzymatic production of lactulose and galacto-oligosaccharides from lactose and fructose using two different commercial β-galactosidases. The methods are generally carried out in a solution of permeate after ultrafiltration of whey. However, the methods disclosed therein use a minimum of 100 g/L lactose.

Wang et al. Appl. Microbiol. Biotechnol. 2013, 97, 6167-6180, is a review of the literature relating to enzymatic production of lactulose, referencing the above Adamczak et al. and Guerrero et al. publications.

The prior art methods therefore teach that yields of lactulose achievable using such enzymatic methods are poor, especially at low fructose concentrations. Although yields can be improved at higher fructose concentrations, such high concentrations result in a product exceeding commercially acceptable sweetness levels. There is therefore a need in the art for an enzymatic method for producing lactulose in better yields than have been possible in the prior art, without the need to use high concentrations of fructose.

Lactosucrose, β-D-galactopyranosyl-(1→4)-α-D-glucopyranosyl-(1→2)-β-D-fructofuranose, is another oligosaccharide which is nondigestible by humans and recognized as a prebiotic. Other health benefits such as the prevention of allergic disease, reduction of cancer risk, and enhancement of calcium absorption have also been described (see Taniguchi, Y.; et al. Biosci. Biotechnol. Biochem. 2007, 71, 2766-2773; and Teramoto, F.; et al. J. Nutr. Sci. Vitaminol. 2006, 52, 337-346). Because of the health benefits and favourable characteristics of lactosucrose, its use as a food ingredient has grown rapidly, particularly in Europe and Japan.

Li et al. J. Agric. Food Chem. 2009, 57, describes the enzymatic production of lactosucrose from sucrose and lactose using a β-galactosidase enzyme from Bacillus circulans. However, the methods used in this paper all use a minimum of 30% w/v sucrose and lactose. Furthermore, Li et al. appears to indicate that such high concentrations of sucrose and lactose are necessary to enable the transgalactosylation to work—at lower concentrations of lactose and/or sucrose, hydrolysis of these disaccharides would be expected to predominate.

Han et al., J. Microbial Biotechnol. 2009, 19(10), 1153-1160, also describes the enzymatic production of lactosucrose from sucrose and lactose. However, the enzyme used is a levansucrase, which is a fructosyltransferase enzyme.

Schro\ddot{d}er et al. Tetrahedron 2004, 60, 2601-2608 describes the enzymatic production of β-D-galactopyranosyl-(1→3)-α-D-glucopyranosyl-(1→2)-β-D-fructofuranose, which is a regioisomer of lactosucrose, and other tri- and tetrasaccharides from sucrose and lactose using a 8-galactosidase enzyme from bovine testes. However, the methods described in this document are all carried out at pH 4.3. For similar reasons to those set out above in relation to Mayer et al., a process carried out at this pH would be unsuitable for carrying out in situ in a milk composition.

Farkas et al. Synthesis 2003, 5, 699-706, describes the enzymatic production of lactosucrose and other tri- and tetrasaccharides from sucrose and lactose using a β-galactosidase enzyme from Bacillus circulans. However, the methods used in this paper for generating lactosucrose use a minimum of 0.5 mol/L lactose.

As indicated above, the prior art methods therefore teach that high concentrations of sucrose and lactose are necessary to enable the transgalactosylation to work: at lower concentrations of lactose and/or sucrose, hydrolysis of these disaccharides would be expected to predominate.

### SUMMARY OF THE INVENTION

The invention provides a method of generating a saccharide containing a galactose moiety and a fructose moiety, wherein:

(a) the galactose moiety is linked to the fructose moiety; or
(b) the galactose moiety and the fructose moiety are separated by at least one monosaccharide moiety other than galactose or fructose;
the method comprising:
contacting a first saccharide, said first saccharide containing a galactose moiety, with a second saccharide, said second saccharide containing a fructose moiety,
the first and the second saccharides being different,
in the presence of an enzyme capable of catalysing the transfer of a galactose moiety to the second saccharide containing the fructose moiety,
the method being carried out at a pH of 5.5 to 9.5;
provided that
(i) when the galactose moiety is linked to the fructose moiety, the concentration of the first saccharide is less than 0.43 mol/L and the concentration of the second saccharide is less than 0.8 mol/L; and
(ii) when the galactose moiety and the fructose moiety are separated by at least one monosaccharide moiety other than galactose or fructose, the concentration of the first saccharide and/or the concentration of the second saccharide is less than 0.5 mol/L.

The invention also provides a method of generating a saccharide containing a galactose moiety and a fructose moiety, wherein:

(a) the galactose moiety is linked to the fructose moiety; or
(b) the galactose moiety and the fructose moiety are separated by at least one monosaccharide moiety other than galactose or fructose;
the method comprising:
contacting a first saccharide, said first saccharide containing a galactose moiety, with a second saccharide, said second saccharide containing a fructose moiety,
the first and the second saccharides being different,
in the presence of an enzyme capable of catalysing the transfer of a galactose moiety to the second saccharide containing the fructose moiety,
wherein the enzyme is selected from the group consisting of:
a) a polypeptide comprising an amino acid sequence having at least 90% sequence identity with SEQ ID NO: 1, wherein said polypeptide consists of at most 980 amino acid residues;
b) a polypeptide encoded by a polynucleotide that hybridizes under at least low stringency conditions with:
i) the nucleic acid sequence comprised in SEQ ID NO: 9, encoding the polypeptide of SEQ ID NO: 1; or
ii) the complementary strand of i).

In one aspect, the invention provides a method of generating a saccharide in which a galactose moiety is linked to a fructose moiety, the method comprising:

contacting a first saccharide, said first saccharide containing a galactose moiety, with a second saccharide, said second saccharide containing a fructose moiety,
the first and the second saccharides being different,
in the presence of an enzyme capable of catalysing the transfer of a galactose moiety to the second saccharide containing the fructose moiety,
the method being carried out at a pH of 5.5 to 9.5;
wherein:
the concentration of the first saccharide is less than 0.43 mol/L; and
the concentration of the second saccharide is less than 0.8 mol/L.

In one aspect, the invention provides a method of generating a saccharide in which a galactose moiety is linked to a fructose moiety, the method comprising:

contacting a first saccharide, said first saccharide containing a galactose moiety, with a second saccharide, said second saccharide containing a fructose moiety,
the first and the second saccharides being different,
in the presence of an enzyme capable of catalysing the transfer of a galactose moiety to the second saccharide containing the fructose moiety,
wherein the concentration of the second saccharide is from 0.083 to 0.472 mol/L.

In another aspect, there is provided a method of generating a saccharide in which a galactose moiety is linked to a fructose moiety, the method comprising:

contacting a first saccharide, said first saccharide containing a galactose moiety, with a second saccharide, said second saccharide containing a fructose moiety,
the first and the second saccharides being different,
in the presence of an enzyme capable of catalysing the transfer of a galactose moiety to the second saccharide containing the fructose moiety,
wherein the enzyme is selected from the group consisting of:
a) a polypeptide comprising an amino acid sequence having at least 90% sequence identity with SEQ ID NO: 1, wherein said polypeptide consists of at most 980 amino acid residues;
b) a polypeptide encoded by a polynucleotide that hybridizes under at least low stringency conditions with:
i) the nucleic acid sequence comprised in SEQ ID NO: 9, encoding the polypeptide of SEQ ID NO: 1; or
ii) the complementary strand of i).

In a further aspect, there is provided a lactulose-containing composition obtainable by the method of the invention.

In a further aspect, there is provided use of an enzyme, as defined above, to generate lactulose. The enzyme can also be used to generate other saccharides in which a galactose moiety is linked to a fructose moiety.

In one aspect, there is provided a method of generating a saccharide containing a galactose moiety and a fructose moiety, the galactose moiety and the fructose moiety being separated by at least one monosaccharide moiety other than galactose or fructose, the method comprising:

contacting a first saccharide, said first saccharide containing a galactose moiety, with a second saccharide, said second saccharide containing a fructose moiety,
the first and the second saccharides being different,
in the presence of an enzyme capable of catalysing the transfer of a galactose moiety to the second saccharide containing the fructose moiety,
wherein the concentration of the first saccharide and/or the concentration of the second saccharide is less than 0.5 mol/L.

In another aspect, there is provided a method of generating a saccharide containing a galactose moiety and a fructose moiety, the galactose moiety and the fructose moiety being separated by at least one monosaccharide moiety other than galactose or fructose, the method comprising:

contacting a first saccharide, said first saccharide containing a galactose moiety, with a second saccharide, said second saccharide containing a fructose moiety,
the first and the second saccharides being different,
in the presence of an enzyme capable of catalysing the transfer of a galactose moiety to the second saccharide containing the fructose moiety,
wherein the enzyme is selected from the group consisting of:
a) a polypeptide comprising an amino acid sequence having at least 90% sequence identity with SEQ ID NO: 1, wherein said polypeptide consists of at most 980 amino acid residues;
b) a polypeptide encoded by a polynucleotide that hybridizes under at least low stringency conditions with:
i) the nucleic acid sequence comprised in SEQ ID NO: 9, encoding the polypeptide of SEQ ID NO: 1; or
ii) the complementary strand of i).

In a further aspect, there is provided use of an enzyme, as defined above, to generate lactosucrose.

### Advantages and Surprising Findings

The inventors have surprisingly found that saccharides in which a galactose moiety is linked to a fructose moiety, in particular lactulose, can be generated from galactose-containing saccharides, in particular lactose, and fructose-containing saccharides, in particular fructose, in high yield even at low concentrations of the fructose-containing saccharide. This is contrary to the teachings of the prior art as fructose is generally known not to be a good acceptor in enzymatic saccharide condensation reactions. In particular, it would have been expected that at low fructose concentrations, hydrolysis of lactose and/or generation of galactooligosaccharides would be the predominant reaction catalysed by the enzyme, and it would have been considered necessary for fructose to be present in high concentrations (resulting in a product exceeding commercially acceptable sweetness levels) to drive the equilibrium towards the formation of lactulose.

The inventors have also surprisingly found that saccharides containing a galactose moiety and a fructose moiety, in which at least one other monosaccharide moiety separates the galactose and fructose moieties, in particular lactosucrose, can be generated from galactose-containing saccharides, in particular lactose, and fructose-containing saccharides, in particular sucrose, in high yield even at low concentrations of either saccharide. This is contrary to the teachings of the prior art as it would have been expected that at low lactose and/or sucrose concentrations, hydrolysis of lactose and/or sucrose would be the predominant reaction catalysed by the enzyme, and it would have been considered necessary for lactose or sucrose to be present in high concentrations to drive the equilibrium towards the formation of lactosucrose.

### BRIEF DESCRIPTION OF THE FIGURES

FIG. 1 illustrates the amount of lactulose produced by the method of the invention starting with 4.8% (w/v) lactose and various concentrations of fructose;

FIG. 2 illustrates the amount of lactulose produced by the method of the invention starting with 7.0% (w/v) lactose and various concentrations of fructose;

FIG. 3 illustrates the amount of lactulose produced by the method of the invention starting with 9.0% (w/v) lactose and various concentrations of fructose; and

FIG. 4 is a chromatogram of enzymatically generated sugar mixtures after 4 hours of reaction, 1: lactose at 30.3 min; 2: 4-lactulose at 32.1 min; 3: lactulose isomer reaction product 34.5 min, 4: glucose at 36.7 and 5: galactose, fructose at 39.7 min.

FIG. 5 is an example of an extracted ion chromatogram (EIC) of a 100 μg/ml solution of lactosucrose generated by the method described in Example 3, showing unlabelled lactosucrose (Hex-DP3) (top, black) followed by EICs of galacto¹³C₁₂-Hex-DP3-DP6 oligomers (lactosucrose Gal-Glu-Fru, galactosyl-lactosucrose Gal-Gal-Glu-Fru, digalactosyl-lactosucrose Gal-Gal-Gal-Glu-Fru and trigalactosyl-lactosucrose Gal-Gal-Gal-Gal-Glu-Fru) in a sample taken after 2 hours biotransformation and diluted 10×; and

FIG. 6 shows combined extracted ion chromatograms of ¹³C₁₂-Hex-DP3-6 oligomers showing the change in profile during biotransformation time with inserts of 9.3-13.0 min showing the maximum presence of larger than ¹³C₁₂-Hex-DP3 oligomers in the t=2 h sample and decreasing amounts in the following samples.

### SEQUENCE LISTINGS

SEQ ID NO: 1 (also named (BIF_917) herein) is a 887 amino acid truncated fragment of SEQ ID NO: 22.
SEQ ID NO: 2 (also named (BIF_995) herein) is a 965 amino acid truncated fragment of SEQ ID NO: 22.
SEQ ID NO: 3 (also named (BIF_1068) herein) is a 1038 amino acid truncated fragment of SEQ ID NO: 22.
SEQ ID NO: 4 (also named (BIF_1172) herein) is a 1142 amino acid truncated fragment of SEQ ID NO: 22.
SEQ ID NO: 5 (also named (BIF_1241) herein) is a 1211 amino acid truncated fragment of SEQ ID NO: 22.
SEQ ID NO: 6 (also named (BIF_1326) herein) is a 1296 amino acid truncated fragment of SEQ ID NO: 22.
SEQ ID NO: 7 is Bifidobacterium bifidum glycoside hydrolase catalytic core
SEQ ID NO: 8 is a nucleotide sequence encoding an extracellular lactase from Bifidobacterium bifidum DSM20215
SEQ ID NO: 9 is nucleotide sequence encoding BIF_917
SEQ ID NO: 10 is nucleotide sequence encoding BIF_995
SEQ ID NO: 11 is nucleotide sequence encoding BIF 1068
SEQ ID NO: 12 is nucleotide sequence encoding BIF_1172
SEQ ID NO: 13 is nucleotide sequence encoding BIF_1241
SEQ ID NO: 14 is nucleotide sequence encoding BIF_1326
SEQ ID NO: 15 is forward primer for generation of above BIF variants
SEQ ID NO: 16 is reverse primer for BIF917
SEQ ID NO: 17 is reverse primer for BIF995
SEQ ID NO: 18 is reverse primer for BIF1068
SEQ ID NO: 19 is reverse primer for BIF1241
SEQ ID NO: 20 is reverse primer for BIF1326
SEQ ID NO: 21 is reverse primer for BIF1478
SEQ ID NO: 22 is extracellular lactase from Bifidobacterium bifidum DSM20215.
SEQ ID NO: 23 is signal sequence of extracellular lactase from Bifidobacterium bifidum DSM20215

### DETAILED DESCRIPTION

The method of the present invention generally comprises contacting a first saccharide, which contains a galactose moiety with a second, different saccharide, containing a fructose moiety, such that a galactose moiety is transferred from the first to the second saccharide to generate a product saccharide containing a galactose moiety and a fructose moiety. In one embodiment, the galactose moiety is linked to the fructose moiety. In another embodiment, the galactose moiety and the fructose moiety being separated by at least one monosaccharide moiety other than galactose or fructose (including but not limited to glucose). In the following paragraphs the first saccharide is also referred to as the “donor” and the second saccharide the “acceptor”.

### Saccharides

In this specification the term ‘saccharide’ in its broadest sense is intended to cover all saccharides (sugars), including naturally occurring and synthetic and semi-synthetic saccharides. The term encompasses monosaccharides (i.e., saccharides that cannot be hydrolyzed into simpler sugars), disaccharides (i.e. compounds having two monosaccharide units (moieties) joined together by a glycoside bond), oligosaccharides (i.e., compounds having 3 to 10 monosaccharide units joined together by glycoside bonds in a branched or unbranched chain or a ring (optionally having a saccharide side chain). and polysaccharides, i.e., compounds having over 10 monosaccharide units joined together by a glycoside bond in a branched or unbranched chain or a ring (optionally having a saccharide side chain).

The saccharide may be bonded to other molecules, such as biomolecules, for example peptides/proteins, lipids and nucleic acids. However, it is preferred for the purposes of the present invention that the saccharide is formed from monosaccharide units only.

In one embodiment, the saccharide is a monosaccharide, i.e., a saccharide that cannot be hydrolyzed into a simpler sugar. The monosaccharide may have the D- or L-configuration, and may be an aldose or ketose. Examples of monosaccharides include hexoses, including aldohexoses such as glucose, galactose, allose, altrose, mannose, gulose, idose and talose and ketohexoses such as fructose, tagatose, psicose and sorbose, and pentoses, examples of which include aldopentoses such as ribose, arabinose, xylose and lyxose and ketopentoses such as ribulose and xylulose.

In an alternative embodiment, the saccharide is a higher saccharide, i.e., a saccharide comprising more than one monosaccharide moiety joined together by glycoside bonds and which are generally hydrolysable into their constituent monosaccharides. Examples of such higher saccharides include disaccharides (2 monosaccharide moieties), oligosaccharides (3 to 10 monosaccharide moieties) and polysaccharides (more than 10 monosaccharide moieties). In this regard, the monosaccharide moieties which form the higher saccharide may be the same or different, and may each independently have the D- or L-configuration, and may each independently be aldose or ketose moieties.

The monosaccharide units which form the higher saccharide may have the same or different numbers of carbon atoms. In one embodiment, the monosaccharide moieties of the higher saccharide are hexose moieties, examples of which include aldohexoses such as glucose, galactose, allose, altrose, mannose, gulose, idose and talose and ketohexoses such as fructose, tagatose, psicose and sorbose. In another embodiment, the monosaccharide moieties of the higher saccharide are aldopentose moieties such as ribose, arabinose, xylose and lyxose and ketopentoses such as ribulose and xylulose.

The monosaccharide moieties which form the higher saccharide are joined together by glycoside bonds. When the monosaccharide moieties are hexose moieties, the glycoside bonds may be 1,4′-glycoside bonds (which may be 1,4′-α- or 1,4′β-glycoside bonds), 1,6′-glycoside bonds (which may be 1,6′-α- or 1,6′β-glycoside bonds), 1,2′-glycoside bonds (which may be 1,2′-α- or 1,2′β-glycoside bonds), or 1,3′-glycoside bonds (which may be 1,3′-α- or 1,3′β-glycoside bonds), or any combination thereof.

In one embodiment, the higher saccharide comprises 2 monosaccharide units (i.e., is a disaccharide). Examples of disaccharides include lactose, galactobiose, maltose, cellobiose, sucrose, trehalose, isomaltulose and trehalulose. In another embodiment, the higher saccharide comprises 3 to 10 monosaccharide units (i.e., is an oligosaccharide).

### First Saccharide (Donor)

The first saccharide may be any saccharide which contains a galactose moiety capable of being transferred to a fructose-containing saccharide. In the present invention, the first saccharide is a higher saccharide in which the galactose moiety to be transferred is linked by a glycosidic bond to one or more other monosaccharide moieties (as defined and exemplified above).

In one embodiment, the first saccharide is lactose.

In one embodiment, the first saccharide is a galactooligosaccharide. Galactooligosaccharides (GOS) consist of short chains of galactose moieties (typically 2 to 10 galactose moieties) linked by glycosidic bonds. In one embodiment, the GOS may solely comprise galactose moieties, i.e. have the general formula (Gal)ₙ where n is typically 2 to 10, such as 2, 3, 4, 5, 6, 7, 8, 9 or 10) examples include galactobiose (Gal-Gal), galactotriose (Gal-Gal-Gal), galactotetraose (Gal-Gal-Gal-Gal), galactopentaose (Gal-Gal-Gal-Gal-Gal), and the like. In one embodiment, the GOS may comprise a chain of galactose moieties terminating with a different monosaccharide moiety (as defined and exemplified above), especially a glucose moiety, i.e. having the general formula (Gal)ₙ-Glu where n is typically 2 to 10, such as 2, 3, 4, 5, 6, 7, 8, 9 or 10): specific examples include galactobiosylglucose (Gal-Gal-Glu), galactotriosylglucose (Gal-Gal-Gal-Glu), and the like.

The first saccharide is present in an amount sufficient to allow a measurable quantity of the galactose moiety to be transferred. The precise concentration varies depending on the nature of the first saccharide. In some embodiments (in particular, those embodiments where the galactose moiety and the fructose moiety are linked in the final product), the concentration of the first saccharide is from 0.01 to 10 mol/L; such as 0.02 to 5 mol/L; such as 0.05 to 2 mol/L; such as 0.1 to 1 mol/L such as less than 0.5 mol/L, such as less than 0.49 mol/L, such as less than 0.48 mol/L, such as less than 0.47 mol/L, such as less than 0.46 mol/L, such as less than 0.45 mol/L, such as less than 0.44 mol/L, such as less than 0.43 mol/L, such as less than 0.42 mol/L, such as less than 0.41 mol/L, such as less than 0.4 mol/L, such as less than 0.39 mol/L, such as less than 0.38 mol/L, such as less than 0.37 mol/L, such as less than 0.36 mol/L, such as less than 0.35 mol/L, such as less than 0.34 mol/L, such as less than 0.33 mol/L, such as less than 0.32 mol/L, such as less than 0.31 mol/L, such as less than 0.3 mol/L, such as 0.088 to 0.380 mol/L, such as 0.117 to 0.292 mol/L, such as 0.132 to 0.277 mol/L, such as 0.132 to 0.160 mol/L, such as 0.190 to 0.220 mol/L, such as 0.249 to 0.277 mol/L.

In some embodiments (in particular, those embodiments wherein the galactose moiety and the fructose moiety are separated by at least one monosaccharide moiety other than galactose or fructose in the final product), the concentration of the first saccharide is less than 0.5 mol/L; such as 0.001 to 0.5 mol/L; such as 0.005 to 0.4 mol/L; such as 0.01 to 0.25 mol/L; such as 0.05 to 0.2 mol/L, such as 0.1 to 0.15 mol/L.

In one embodiment, (in particular, those embodiments where the galactose moiety and the fructose moiety are linked in the final product) the first saccharide is lactose and the concentration of the lactose is less than 171.2 g/L (0.5 mol/L), such as less than 167.7 g/L (0.49 mol/L), such as less than 164.3 g/L (0.48 mol/L), such as less than 160.9 g/L (0.47 mol/L), such as less than 157.5 g/L (0.46 mol/L), such as less than 154.0 g/L (0.45 mol/L), such as less than 150.6 g/L (0.44 mol/L), such as less than 147.2 g/L (0.43 mol/L), such as less than 143.8 g/L (0.42 mol/L), such as less than 140.3 g/L (0.41 mol/L), such as less than 136.2 g/L (0.4 mol/L), such as less than 133.5 g/L (0.39 mol/L), such as less than 130.1 g/L (0.38 mol/L), such as less than 126.7 g/L (0.37 mol/L), such as less than 123.3 g/L (0.36 mol/L), such as less than 119.8 g/L (0.35 mol/L), such as less than 116.4 g/L (0.34 mol/L), such as less than 113.0 g/L (0.33 mol/L), such as less than 109.5 g/L (0.32 mol/L), such as less than 106.1 g/L (0.31 mol/L), such as less than 102.7 g/L (0.3 mol/L), such as from 30 to 130 g/L (0.088 to 0.380 mol/L), such as 40 to 100 g/L (0.117 to 0.292 mol/L), such as 45 to 95 g/L (0.132 to 0.277 mol/L), such as 45 to 55 g/L (0.132 to 0.160 mol/L), such as 65 to 75 g/L (0.190 to 0.220 mol/L), such as 85 to 95 g/L (0.249 to 0.277 mol/L).

In another embodiment (in particular, those embodiments wherein the galactose moiety and the fructose moiety are separated by at least one monosaccharide moiety other than galactose or fructose in the final product), the first saccharide is lactose and the concentration of the lactose is less than 171.2 g/L (0.5 mol/L), such as less than 167.7 g/L (0.49 mol/L), such as less than 164.3 g/L (0.48 mol/L), such as less than 160.9 g/L (0.47 mol/L), such as less than 157.5 g/L (0.46 mol/L), such as less than 154.0 g/L (0.45 mol/L), such as less than 150.6 g/L (0.44 mol/L), such as less than 147.2 g/L (0.43 mol/L), such as less than 143.8 g/L (0.42 mol/L), such as less than 140.3 g/L (0.41 mol/L), such as less than 136.2 g/L (0.4 mol/L), such as less than 133.5 g/L (0.39 mol/L), such as less than 130.1 g/L (0.38 mol/L), such as less than 126.7 g/L (0.37 mol/L), such as less than 123.3 g/L (0.36 mol/L), such as less than 119.8 g/L (0.35 mol/L), such as less than 116.4 g/L (0.34 mol/L), such as less than 113.0 g/L (0.33 mol/L), such as less than 109.5 g/L (0.32 mol/L), such as less than 106.1 g/L (0.31 mol/L), such as less than 102.7 g/L (0.3 mol/L), such as 0.001 to 0.5 mol/L; such as 0.005 to 0.4 mol/L; such as 0.01 to 0.25 mol/L; such as 0.05 to 0.2 mol/L, such as 0.1 to 0.15 mol/L.

### Second Saccharide (Acceptor)

The second saccharide may be any saccharide which contains a fructose moiety and is capable of accepting a galactose moiety from the first saccharide. The second saccharide may be fructose or a higher saccharide in which the fructose moiety which accepts the galactose moiety being transferred is linked by a glycosidic bond to one or more other monosaccharide moieties (as defined and exemplified above).

In one embodiment, the second saccharide is fructose. In one embodiment, the first saccharide is lactose and the second saccharide is fructose, so that the saccharide generated is lactulose.

In one embodiment, the second saccharide is sucrose. In one embodiment, the first saccharide is lactose and the second saccharide is sucrose, so that the saccharide generated is lactosucrose.

In one embodiment, the first saccharide is lactose and the second saccharide is lactulose, so that the saccharide generated is galactosyl-lactulose (Gal-Gal-Fru). In one embodiment, the first saccharide is lactose and the second saccharide is galactosyl-lactulose, so that the saccharide generated is of the formula Gal-Gal-Gal-Fru. This can be repeated to provide a galactooligosaccharide (as defined above) with up to 10 galactose moieties terminating with a fructose moiety.

In one embodiment, the first saccharide is lactose and the second saccharide is lactosucrose, so that the saccharide generated is galactosyl-lactosucrose (Gal-Gal-Glu-Fru). In one embodiment, the first saccharide is lactose and the second saccharide is galactosyl-lactosucrose, so that the saccharide generated is digalactosyl-lactosucrose (Gal-Gal-Gal-Glu-Fru). In one embodiment, the first saccharide is lactose and the second saccharide is digalactosyl-lactosucrose, so that the saccharide generated is trigalactosyl-lactosucrose (Gal-Gal-Gal-Gal-Glu-Fru). This can be repeated to provide a galactooligosaccharide (as defined above) with up to 10 galactose moieties terminating with a glucose moiety linked to a fructose moiety.

In one embodiment, the second saccharide is a fructo-oligosaccharide (FOS). FOS consist of short chains of fructose molecules which may optionally terminate with another monosaccharide moiety, especially glucose moiety. In one embodiment, the FOS may solely comprise fructose moieties, i.e. have the general formula (Fru)ₙ where n is typically 2 to 7, such as 2, 3, 4, 5, 6, 7) examples include inulobiose (Fru-Fru), inulotriose (Fru-Fru), and inulotetraose (Fru-Fru-Fru-Fru). Such fructooliogosaccharides are typically produced by degradation of inulin.

In one embodiment, the FOS may comprise a chain of fructose moieties terminating with a different monosaccharide moiety (as defined and exemplified above), especially a glucose moiety, such as those having the general formula Glu-(Fru)ₙ where n is typically 1 to 7, such as 1, 2, 3, 4, 5, 6 or 7, 8, 9 or 10): specific examples include sucrose (Glu-Fru), kestose (Glu-Fru-Fru), nystose (Glu-Fru-Fru-Fru), fructosylnystose (Glu-Fru-Fru-Fru-Fru) and the like. In this embodiment, the method of the invention may cause the galactose moiety to form a bond to either the glucose or the fructose moiety. Preferably, the method of the invention causes the galactose moiety to form a bond to the glucose moiety.

The second saccharide is present in an amount sufficient to allow a measurable quantity of the galactose moiety to be transferred. The precise concentration varies depending on the nature of the second saccharide. In some embodiments, (in particular, those embodiments where the galactose moiety and the fructose moiety are linked in the final product), the concentration of the second saccharide is from 0.01 to 10 mol/L; such as 0.02 to 5 mol/L; such as 0.05 to 2 mol/L; such as 0.1 to 1 mol/L; such as 0.02 to 0.5 mol/L. In some embodiments, the concentration of the second saccharide is less than 0.8 mol/L, such as less than 0.79 mol/L, such as less than 0.78 mol/L, such as less than 0.77 mol/L, such as less than 0.76 mol/L, such as less than 0.75 mol/L, such as less than 0.74 mol/L, such as less than 0.73 mol/L, such as less than 0.72 mol/L, such as less than 0.71 mol/L, such as less than 0.7 mol/L, such as less than 0.69 mol/L, such as less than 0.68 mol/L, such as less than 0.67 mol/L, such as less than 0.66 mol/L, such as less than 0.65 mol/L, such as less than 0.64 mol/L, such as less than 0.63 mol/L, such as less than 0.62 mol/L, such as less than 0.61 mol/L, such as less than 0.6 mol/L, such as less than 0.59 mol/L, such as less than 0.58 mol/L, such as less than 0.57 mol/L, such as less than 0.56 mol/L, such as less than 0.55 mol/L, such as less than 0.54 mol/L, such as less than 0.53 mol/L, such as less than 0.52 mol/L, such as less than 0.51 mol/L, such as less than 0.5 mol/L, such as less than 0.49 mol/L, such as less than 0.48 mol/L, such as less than 0.47 mol/L, such as less than 0.46 mol/L, such as less than 0.45 mol/L, such as less than 0.44 mol/L, such as less than 0.43 mol/L, such as less than 0.42 mol/L, such as less than 0.41 mol/L, such as less than 0.4 mol/L, such as less than 0.39 mol/L, such as less than 0.38 mol/L, such as less than 0.37 mol/L, such as less than 0.36 mol/L, such as less than 0.35 mol/L, such as less than 0.34 mol/L, such as less than 0.33 mol/L, such as less than 0.32 mol/L, such as less than 0.31 mol/L, such as less than 0.3 mol/L. In some embodiments, the concentration of the second saccharide is more than 0.1 mol/L, such as more than 0.11 mol/L, such as more than 0.12 mol/L, such as more than 0.13 mol/L, such as more than 0.14 mol/L, such as more than 0.15 mol/L, such as more than 0.16 mol/L, such as more than 0.17 mol/L, such as more than 0.18 mol/L, such as more than 0.19 mol/L, such as more than 0.2 mol/L such as more than 0.21 mol/L, such as more than 0.22 mol/L, such as more than 0.23 mol/L, such as more than 0.24 mol/L, such as more than 0.25 mol/L, such as more than 0.26 mol/L, such as more than 0.27 mol/L. In some embodiments, the concentration of the second saccharide is 0.083 to 0.472 mol/L. In some embodiments, the concentration of the second saccharide is 0.278 to 0.444 mol/L.

In some embodiments of the present invention, (in particular, those embodiments where the galactose moiety and the fructose moiety are linked in the final product), the second saccharide is fructose and the concentration of the fructose is less than 0.8 mol/L, such as less than 0.79 mol/L, such as less than 0.78 mol/L, such as less than 0.77 mol/L, such as less than 0.76 mol/L, such as less than 0.75 mol/L, such as less than 0.74 mol/L, such as less than 0.73 mol/L, such as less than 0.72 mol/L, such as less than 0.71 mol/L, such as less than 0.7 mol/L, such as less than 0.69 mol/L, such as less than 0.68 mol/L, such as less than 0.67 mol/L, such as less than 0.66 mol/L, such as less than 0.65 mol/L, such as less than 0.64 mol/L, such as less than 0.63 mol/L, such as less than 0.62 mol/L, such as less than 0.61 mol/L, such as less than 0.6 mol/L, such as less than 0.59 mol/L, such as less than 0.58 mol/L, such as less than 0.57 mol/L, such as less than 0.56 mol/L, such as less than 0.55 mol/L, such as less than 0.54 mol/L, such as less than 0.53 mol/L, such as less than 0.52 mol/L, such as less than 0.51 mol/L, such as less than 0.5 mol/L, such as less than 0.49 mol/L, such as less than 0.48 mol/L, such as less than 0.47 mol/L, such as less than 0.46 mol/L, such as less than 0.45 mol/L, such as less than 0.44 mol/L, such as less than 0.43 mol/L, such as less than 0.42 mol/L, such as less than 0.41 mol/L, such as less than 0.4 mol/L, such as less than 0.39 mol/L, such as less than 0.38 mol/L, such as less than 0.37 mol/L, such as less than 0.36 mol/L, such as less than 0.35 mol/L, such as less than 0.34 mol/L, such as less than 0.33 mol/L, such as less than 0.32 mol/L, such as less than 0.31 mol/L, such as less than 0.3 mol/L. In some embodiments, the concentration of the fructose is 15 to 85 g/L (0.083 to 0.472 mol/L), such as 20 to 80 g/L (0.111 to 0.444 mol/L), such as 45 to 85 g/L (0.25 to 0.472 mol/L), such as 50 to 80 g/L (0.278 to 0.444 mol/L), such as 45 to 55 g/L (0.25 to 0.306 mol/L), such as 55 to 65 g/L (0.306 to 0.361 mol/L), such as 65 to 75 g/L (0.361 to 0.417 mol/L), such as 75 to 85 g/L (0.417 to 0.472 mol/L). It has been surprisingly found that enzymatic transfer of a galactose moiety to the fructose can take place even at this low concentration of the fructose. This is contrary to the teachings of the prior art as it would have been expected that at low concentrations of the fructose, other reactions (typically hydrolysis of the first saccharide and/or generation of galactooligosaccharides) would be the predominant reaction catalysed by the enzyme.

In some embodiments (in particular, those embodiments wherein the galactose moiety and the fructose moiety are separated by at least one monosaccharide moiety other than galactose or fructose in the final product), the concentration of the second saccharide is less than 0.5 mol/L; such as 0.001 to 0.5 mol/L; such as 0.005 to 0.4 mol/L, such as 0.01 to 0.35 mol/L, such as 0.1 to 0.3 mol/L, such as 0.15 to 0.2 mol/L.

In some embodiments (in particular, those embodiments wherein the galactose moiety and the fructose moiety are separated by at least one monosaccharide moiety other than galactose or fructose in the final product), the second saccharide is sucrose and the concentration of the sucrose is less than 0.5 mol/L, less than 171.2 g/L (0.5 mol/L), such as less than 167.7 g/L (0.49 mol/L), such as less than 164.3 g/L (0.48 mol/L), such as less than 160.9 g/L (0.47 mol/L), such as less than 157.5 g/L (0.46 mol/L), such as less than 154.0 g/L (0.45 mol/L), such as less than 150.6 g/L (0.44 mol/L), such as less than 147.2 g/L (0.43 mol/L), such as less than 143.8 g/L (0.42 mol/L), such as less than 140.3 g/L (0.41 mol/L), such as less than 136.2 g/L (0.4 mol/L), such as less than 133.5 g/L (0.39 mol/L), such as less than 130.1 g/L (0.38 mol/L), such as less than 126.7 g/L (0.37 mol/L), such as less than 123.3 g/L (0.36 mol/L), such as less than 119.8 g/L (0.35 mol/L), such as less than 116.4 g/L (0.34 mol/L), such as less than 113.0 g/L (0.33 mol/L), such as less than 109.5 g/L (0.32 mol/L), such as less than 106.1 g/L (0.31 mol/L), such as less than 102.7 g/L (0.3 mol/L). In some embodiments, the concentration of the sucrose is more than 3.4 g/L (0.01 mol/L), such as more than 6.8 g/L (0.02 mol/L), such as more than 10.3 g/L (0.03 mol/L), such as more than 13.7 g/L (0.04 mol/L), such as more than 17.1 g/L (0.05 mol/L), such as more than 20.5 g/L (0.06 mol/L), such as more than 24.0 g/L (0.07 mol/L), such as more than 27.3 g/L (0.08 mol/L), such as more than 30.8 g/L (0.09 mol/L), such as more than 34.2 g/L (0.1 mol/L), such as more than 37.7 g/L (0.11 mol/L), such as more than 41.1 g/L (0.12 mol/L), such as more than 44.5 g/L (0.13 mol/L), such as more than 47.9 g/L (0.14 mol/L), such as more than 51.3 g/L (0.15 mol/L), such as 0.001 to 0.5 mol/L; such as 0.005 to 0.4 mol/L, such as 0.01 to 0.35 mol/L, such as 0.1 to 0.3 mol/L, such as 0.15 to 0.2 mol/L.

### Enzyme

The enzyme used in the present invention is not particularly limited, provided it is capable of catalysing the transfer of a galactose moiety from the galactose-containing first saccharide (especially lactose) to the fructose-containing second saccharide (especially fructose). Enzymes capable of catalysing transfer of a galactose moiety from a galactosyl-containing saccharide to a molecule other than water (particularly to a second saccharide) are generally referred to as “transgalactosylases”. The transgalactosylating activity can be measured by means of HPLC quantification or enzymatic assays as described in WO 2013/182686.

The enzyme may have other side activities in addition to its transgalactosylase activity. Typical side activities include saccharide hydrolase activity (e.g. ability to hydrolyse glycosidic bonds in a saccharide, especially the galactose-containing first saccharide and/or the fructose-containing second saccharide); protease activity; lipase activity; phospholipase activity. Preferably, the relative transgalactosylase activity of the enzyme comprises at least 50%, such as at least 60%, such as at least 70%, such as at least 75%, such as at least 80%, such as at least 85%, such as at least 90%, such as at least 95%, such as at least 97%, such as at least 98%, such as at least 99% of the total activity of the enzyme. In the present context, the term “transgalactosylating activity” means the transfer of a galactose moiety to a molecule other than water. The activity can be measured as [glucose]−[galactose] generated at any given time during reaction or by direct quantification of the GOS generated at any given time during the reaction. The relative transgalactosylase activity may then be calculated as ([glucose]−[galactose])/[glucose]×100, Means to measure glucose and galactose concentration is known to a person skilled in the art or in WO 2013/182686.

In one embodiment, the enzyme is a β-galactosidase. A β-galactosidase is a hydrolase enzyme that catalyzes the hydrolysis of β-galactosides into monosaccharides. Such enzymes are typically classified in Enzyme Classification (E.C.) 3.2.1.23.

In one embodiment, the enzyme is of bacterial origin or fungal origin. In one embodiment, the enzyme is of bacterial origin. In one embodiment, the enzyme is of Bifidobacteria origin. In one embodiment, the enzyme is of Bifidobacterium bifidum origin.

In one embodiment, the enzyme is selected from the group consisting of:

a) a polypeptide having transgalactosylating activity comprising an amino acid sequence having at least 90% sequence identity with SEQ ID NO: 1, wherein said polypeptide consists of at most 980 amino acid residues
b) a polypeptide encoded by a polynucleotide that hybridizes under at least low stringency conditions with: i) the nucleic acid sequence comprised in SEQ ID NO: 9, encoding the polypeptide of SEQ ID NO: 1; or ii) the complementary strand of i). Such enzymes are disclosed generally and specifically in WO 2013/186286.

In one embodiment (particularly, those embodiments where the galactose moiety and the fructose moiety are linked in the final product, such as lactulose), the concentration of enzyme is suitably 500 to 10,000 units of enzyme activity (U) per kg of the composition in which the transgalactosylation reaction takes place. Preferably, the concentration of enzyme is 1000 to 5000 units of enzyme activity (U) per kg of the composition. The units of activity of this enzyme are measured according to the assay disclosed in WO 2013/186286 as Method 4 and reproduced herein as Example 4, Method 4.

In one embodiment, when the reaction is carried out in situ in a milk composition, the concentration of enzyme is suitably 500 to 10,000 units of enzyme activity (U) per litre of the milk composition. Preferably, the concentration of enzyme is 1000 to 5000 units of enzyme activity (U) per litre of the milk composition.

In one embodiment, (in particular, those embodiments where the galactose moiety and the fructose moiety are separated by at least one monosaccharide moiety other than galactose or fructose in the final product, such as lactosucrose) the concentration of enzyme is suitably 500 to 10,000 units of enzyme activity (U) per kg of the composition in which the transgalactosylation reaction takes place. Preferably, the concentration of enzyme is 1000 to 5000 units of enzyme activity (U) per kg of the composition. The units of activity of this enzyme are measured according to the assay disclosed in WO 2013/186286 as Method 4 and reproduced herein as Example 4, Method 4.

In one embodiment, when the reaction is carried out in situ in a milk composition, the concentration of enzyme is suitably 500 to 10,000 units of enzyme activity (U) per litre of the milk composition. Preferably, the concentration of enzyme is 1000 to 5000 units of enzyme activity (U) per litre of the milk composition.

In one embodiment (particularly, those embodiments where the galactose moiety and the fructose moiety are linked in the final product, such as lactulose), the concentration of enzyme is suitably 0.2 to 4 g of pure enzyme protein per kg of the composition in which the transgalactosylation reaction takes place. Preferably, the concentration of enzyme is 0.4 to 2 g of pure enzyme protein per kg of the composition.

In this embodiment, when the reaction is carried out in situ in a milk composition, the concentration of enzyme is suitably 0.2 to 4 g of pure enzyme protein per litre of the milk composition. Preferably, the concentration of enzyme is 0.4 to 2 g of pure enzyme protein per litre of the milk composition.

In one embodiment, (in particular, those embodiments where the galactose moiety and the fructose moiety are separated by at least one monosaccharide moiety other than galactose or fructose in the final product, such as lactosucrose) the concentration of enzyme is suitably 0.2 to 4 g of pure enzyme protein per kg of the composition in which the transgalactosylation reaction takes place. Preferably, the concentration of enzyme is 0.4 to 2 g of pure enzyme protein per kg of the composition.

In this embodiment, when the reaction is carried out in situ in a milk composition, the concentration of enzyme is suitably 0.2 to 4 g of pure enzyme protein per litre of the milk composition. Preferably, the concentration of enzyme is 0.4 to 2 g of pure enzyme protein per litre of the milk composition.

In one aspect, disclosed herein is a polypeptide having transgalactosylating activity, which comprises an amino acid sequence having at least 90% sequence identity with SEQ ID NO: 1, and wherein said polypeptide, when being an expression product in a suitable host strain (e.g., Bacillus subtilis) comprising a nucleic acid which encodes said polypeptide, is the only polypeptide expression product of said nucleic acid sequence that exhibits transgalactosylating activity.

In one aspect, disclosed herein is a polypeptide having transgalactosylating activity selected from the group consisting of:
a. a polypeptide comprising an amino acid sequence having at least 90% sequence identity with SEQ ID NO: 1, wherein said polypeptide consists of at most 980 amino acid residues,
b. a polypeptide comprising an amino acid sequence having at least 97% sequence identity with SEQ ID NO: 2, wherein said polypeptide consists of at most 975 amino acid residues,
c. a polypeptide comprising an amino acid sequence having at least 96.5% sequence identity with SEQ ID NO: 3, wherein said polypeptide consists of at most 1300 amino acid residues,
d. a polypeptide encoded by a polynucleotide that hybridizes under at least low stringency conditions with i) the nucleic acid sequence comprised in SEQ ID NO: 9, 10, 11, 12 or 13 encoding the polypeptide of SEQ ID NO: 1, 2, 3, 4, or 5; or ii) the complementary strand of i),
e. a polypeptide encoded by a polynucleotide comprising a nucleotide sequence having at least 70% identity to the nucleotide sequence encoding for the polypeptide of SEQ ID NO: 1, 2, 3, 4 or 5 or the nucleotide sequence comprised in SEQ ID NO: 9, 10, 11, 12 or 13 encoding a mature polypeptide, and
f. a polypeptide comprising a deletion, insertion and/or conservative substitution of one or more amino acid residues of SEQ ID NO: 1, 2, 3, 4 or 5.

In another aspect disclosed herein is a polypeptide having transgalactosylating activity selected from the group consisting of:
a. a polypeptide comprising an amino acid sequence having at least 96.5% sequence identity with SEQ ID NO: 3, wherein said polypeptide consists of at most 1300 amino acid residues,
b. a polypeptide comprising an amino acid sequence having at least 90% sequence identity with SEQ ID NO: 1, wherein said polypeptide consists of at most 980 amino acid residues,
c. a polypeptide encoded by a polynucleotide that hybridizes under at least low stringency conditions with i) the nucleic acid sequence comprised in SEQ ID NO: 9, 10, 11, 12 or 13 encoding the polypeptide of SEQ ID NO: 1, 2, 3, 4, or 5; or ii) the complementary strand of i),
d. a polypeptide encoded by a polynucleotide comprising a nucleotide sequence having at least 70% identity to the nucleotide sequence encoding for the polypeptide of SEQ ID NO: 1, 2, 3, 4 or 5 or the nucleotide sequence comprised in SEQ ID NO: 9, 10, 11, 12 or 13 encoding a mature polypeptide, and
e. a polypeptide comprising a deletion, insertion and/or conservative substitution of one or more amino acid residues of SEQ ID NO: 1, 2, 3, 4 or 5.

In one aspect, disclosed herein is polypeptide which is a C-terminal truncated fragment of SEQ ID NO:22 having transgalactosylating activity and which are stable against further truncation such as by proteolytic degradation when produced in a suitable organism such as Bacillus subtilis and/or which are stable against further truncation during storage after final formulation.

In one aspect, disclosed herein is a polypeptide comprising an amino acid sequence having at least 90% sequence identity with SEQ ID NO: 1, wherein said polypeptide consists of at most 980 amino acid residues.

In one aspect, disclosed herein is a polypeptide comprising an amino acid sequence having at least 97% sequence identity with SEQ ID NO: 2, wherein said polypeptide consists of at most 975 amino acid residues, is provided.

In one aspect, disclosed herein is a polypeptide comprising an amino acid sequence having at least 96.5% sequence identity with SEQ ID NO: 3, wherein said polypeptide consists of at most 1300 amino acid residues.

In one aspect, disclosed herein is a nucleic acid capable of encoding a polypeptide as described herein.

In one aspect, disclosed herein is an expression vector and/or a plasmid comprising a nucleic as described herein, or capable of expressing a polypeptide as described herein.

In one aspect, disclosed herein is a cell capable of expressing a polypeptide as described herein.

The term “isolated” means that the polypeptide is at least substantially free from at least one other component with which the sequence is naturally associated in nature and as found in nature. In one aspect, “isolated polypeptide” as used herein refers to a polypeptide which is at least 30% pure, at least 40% pure, at least 60% pure, at least 80% pure, at least 90% pure, and at least 95% pure, as determined by SDS-PAGE.

The term “substantially pure polypeptide” means herein a polypeptide preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of other polypeptide material with which it is natively associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptides disclosed herein are preferably in a substantially pure form. In particular, it is preferred that the polypeptides are in “essentially pure form”, i.e., that the polypeptide preparation is essentially free of other polypeptide material with which it is natively associated. This can be accomplished, for example, by preparing the polypeptide by means of well-known recombinant methods or by classical purification methods. Herein, the term “substantially pure polypeptide” is synonymous with the terms “isolated polypeptide” and “polypeptide in isolated form.”

The term “purified” or “pure” means that a given component is present at a high level state—e.g. at least about 51% pure, such as at least 51% pure, or at least about 75% pure such as at least 75% pure, or at least about 80% pure such as at least 80% pure, or at least about 90% pure such as at least 90% pure, or at least about 95% pure such as at least 95% pure, or at least about 98% pure such as at least 98% pure. The component is desirably the predominant active component present in a composition.

The term “microorganism” in relation to the present invention includes any “microorganism” that could comprise a nucleotide sequence according to the present invention or a nucleotide sequence encoding for a polypeptide having the specific properties as defined herein and/or products obtained therefrom. In the present context, “microorganism” may include any bacterium or fungus being able to ferment a milk substrate.

The term “host cell”—in relation to the present invention includes any cell that comprises either a nucleotide sequence encoding a polypeptide having the specific properties as defined herein or an expression vector as described above and which is used in the production of a polypeptide having the specific properties as defined herein. In one aspect, the production is recombinant production.

In the present context the term “Pfam domains” means regions within a protein sequence that are identified as either Pfam-A or Pfam-B based on multiple sequence alignments and the presence of Hidden Markov Motifs (“The Pfam protein families database”: R. D. Finn, J. Mistry, J. Tate, P. Coggill, A. Heger, J. E. Pollington, Gavin, P. Gunesekaran, G. Ceric, K. Forslund, L. Holm, E. L. Sonnhammer, S. R. Eddy, A. Bateman Nucleic Acids Research (2010) Database Issue 38:D211-222.). As examples of Pfam domains mention may be made of Glyco_hydro2N (PF02837), Glyco_hydro (PF00703), Glyco_hydro 2C (PF02836) and Bacterial Ig-like domain (group 4) (PF07532).

As used herein “a position corresponding to position” means that an alignment as described herein is made between a particular query polypeptide and the reference polypeptide. The position corresponding to a specific position in the reference polypeptide is then identified as the corresponding amino acid in the alignment with the highest sequence identity.

A “variant” or “variants” refers to either polypeptides or nucleic acids. The term “variant” may be used interchangeably with the term “mutant”. Variants include insertions, substitutions, transversions, truncations, and/or inversions at one or more locations in the amino acid or nucleotide sequence, respectively. The phrases “variant polypeptide”, “polypeptide variant”, “polypeptide”, “variant” and “variant enzyme” mean a polypeptide/protein that has an amino acid sequence that either has or comprises a selected amino acid sequence of or is modified compared to the selected amino acid sequence, such as SEQ ID NO: 1, 2, 3, 4 or 5.

As used herein, “reference enzymes,” “reference sequence,” “reference polypeptide” mean enzymes and polypeptides from which any of the variant polypeptides are based, e.g., SEQ ID NO: 1, 2, 3, 4 or 5. A “reference nucleic acid” means a nucleic acid sequence encoding the reference polypeptide.

As used herein, the terms “reference sequence” and “subject sequence” are used interchangeably.

As used herein, “query sequence” means a foreign sequence, which is aligned with a reference sequence in order to see if it falls within the scope of the present invention.

Accordingly, such query sequence can for example be a prior art sequence or a third party sequence.

As used herein, the term “sequence” can either be referring to a polypeptide sequence or a nucleic acid sequence, depending of the context.

As used herein, the terms “polypeptide sequence” and “amino acid sequence” are used interchangeably.

The signal sequence of a “variant” may be the same or may differ from the signal sequence of the wild-type a Bacillus signal peptide or any signal sequence that will secrete the polypeptide. A variant may be expressed as a fusion protein containing a heterologous polypeptide. For example, the variant can comprise a signal peptide of another protein or a sequence designed to aid identification or purification of the expressed fusion protein, such as a His-Tag sequence.

To describe the various variants that are contemplated to be encompassed by the present disclosure, the following nomenclature will be adopted for ease of reference. Where the substitution includes a number and a letter, e.g., 592P, then this refers to {position according to the numbering system/substituted amino acid}. Accordingly, for example, the substitution of an amino acid to proline in position 592 is designated as 592P. Where the substitution includes a letter, a number, and a letter, e.g., D592P, then this refers to {original amino acid/position according to the numbering system/substituted amino acid}.

Accordingly, for example, the substitution of alanine with proline in position 592 is designated as A592P.

Where two or more substitutions are possible at a particular position, this will be designated by contiguous letters, which may optionally be separated by slash marks “/”, e.g., G303ED or G303E/D.

Position(s) and substitutions are listed with reference to for example either SEQ ID NO: 1, 2, 3, 4 or 5. For example equivalent positions in another sequence may be found by aligning this sequence with either SEQ ID NO: 1, 2, 3, 4 or 5 to find an alignment with the highest percent identity and thereafter determining which amino acid aligns to correspond with an amino acid of a specific position of either SEQ ID NO: 1, 2, 3, 4 or 5. Such alignment and use of one sequence as a first reference is simply a matter of routine for one of ordinary skill in the art.

As used herein, the term “expression” refers to the process by which a polypeptide is produced based on the nucleic acid sequence of a gene. The process includes both transcription and translation.

As used herein, “polypeptide” is used interchangeably with the terms “amino acid sequence”, “enzyme”, “peptide” and/or “protein”. As used herein, “nucleotide sequence” or “nucleic acid sequence” refers to an oligonucleotide sequence or polynucleotide sequence and variants, homologues, fragments and derivatives thereof. The nucleotide sequence may be of genomic, synthetic or recombinant origin and may be double-stranded or single-stranded, whether representing the sense or anti-sense strand. As used herein, the term “nucleotide sequence” includes genomic DNA, cDNA, synthetic DNA, and RNA.

“Homologue” means an entity having a certain degree of identity or “homology” with the subject amino acid sequences and the subject nucleotide sequences. In one aspect, the subject amino acid sequence is SEQ ID NO: 1, 2, 3, 4 or 5, and the subject nucleotide sequence preferably is SEQ ID NO: 9, 10, 11, 12 or 13.

A “homologous sequence” includes a polynucleotide or a polypeptide having a certain percent, e.g., 80%, 85%, 90%, 95%, or 99%, of sequence identity with another sequence. Percent identity means that, when aligned, that percentage of bases or amino acid residues are the same when comparing the two sequences. Amino acid sequences are not identical, where an amino acid is substituted, deleted, or added compared to the subject sequence. The percent sequence identity typically is measured with respect to the mature sequence of the subject protein, i.e., following removal of a signal sequence, for example. Typically, homologues will comprise the same active site residues as the subject amino acid sequence. Homologues also retain enzymatic activity, although the homologue may have different enzymatic properties than the wild-type.

As used herein, “hybridization” includes the process by which a strand of nucleic acid joins with a complementary strand through base pairing, as well as the process of amplification as carried out in polymerase chain reaction (PCR) technologies. The variant nucleic acid may exist as single- or double-stranded DNA or RNA, an RNA/DNA heteroduplex or an RNA/DNA copolymer. As used herein, “copolymer” refers to a single nucleic acid strand that comprises both ribonucleotides and deoxyribonucleotides. The variant nucleic acid may be codon-optimized to further increase expression.

As used herein, a “synthetic” compound is produced by in vitro chemical or enzymatic synthesis. It includes, but is not limited to, variant nucleic acids made with optimal codon usage for host organisms, such as a yeast cell host or other expression hosts of choice.

As used herein, “transformed cell” includes cells, including both bacterial and fungal cells, which have been transformed by use of recombinant DNA techniques. Transformation typically occurs by insertion of one or more nucleotide sequences into a cell. The inserted nucleotide sequence may be a heterologous nucleotide sequence, i.e., is a sequence that is not natural to the cell that is to be transformed, such as a fusion protein.

As used herein, “operably linked” means that the described components are in a relationship permitting them to function in their intended manner. For example, a regulatory sequence operably linked to a coding sequence is ligated in such a way that expression of the coding sequence is achieved under condition compatible with the control sequences.

As used herein, the term “fragment” is defined herein as a polypeptide having one or more (several) amino acids deleted from the amino and/or carboxyl terminus wherein the fragment has activity.

In one aspect, the term “fragment” is defined herein as a polypeptide having one or more (several) amino acids deleted from the amino and/or carboxyl terminus of the polypeptide of SEQ ID NO: 1, 2, 3, 4 or 5; wherein the fragment has transgalactosylating activity.

The term “Galactose Binding domain-like” as used herein is abbreviated to and interchangeable with the term “GBD”.

### Degree of Identity

The relatedness between two amino acid sequences or between two nucleotide sequences is described by the parameter “identity”.

In one embodiment, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the reference sequence.

In one embodiment, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the longest of the two sequences.

In another embodiment, the degree of sequence identity between the query sequence and the reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the “alignment length”, where the alignment length is the length of the entire alignment including gaps and overhanging parts of the sequences.

Sequence identity comparisons can be conducted by eye, or more usually, with the aid of readily available sequence comparison programs. These commercially available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalising the insertion of gaps, gap extensions and alignment of non-similar amino acids. The scoring system of the comparison algorithms include:
i) assignment of a penalty score each time a gap is inserted (gap penalty score),
ii) assignment of a penalty score each time an existing gap is extended with an extra position (extension penalty score),
iii) assignment of high scores upon alignment of identical amino acids, and
iv) assignment of variable scores upon alignment of non-identical amino acids.

Most alignment programs allow the gap penalties to be modified. However, it is preferred to use the default values when using such software for sequence comparisons.

The scores given for alignment of non-identical amino acids are assigned according to a scoring matrix also called a substitution matrix. The scores provided in such substitution matrices are reflecting the fact that the likelihood of one amino acid being substituted with another during evolution varies and depends on the physical/chemical nature of the amino acid to be substituted. For example, the likelihood of a polar amino acid being substituted with another polar amino acid is higher compared to being substituted with a hydrophobic amino acid. Therefore, the scoring matrix will assign the highest score for identical amino acids, lower score for non-identical but similar amino acids and even lower score for non-identical non-similar amino acids. The most frequently used scoring matrices are the PAM matrices (Dayhoff et al. (1978), Jones et al. (1992)), the BLOSUM matrices (Henikoff and Henikoff (1992)) and the Gonnet matrix (Gonnet et al. (1992)).

Suitable computer programs for carrying out such an alignment include, but are not limited to, Vector NTI (Invitrogen Corp.) and the ClustalV, ClustalW and ClustalW2 programs (Higgins DG & Sharp PM (1988), Higgins et al. (1992), Thompson et al. (1994), Larkin et al. (2007). A selection of different alignment tools is available from the ExPASy Proteomics server at www.expasy.org. Another example of software that can perform sequence alignment is BLAST (Basic Local Alignment Search Tool), which is available from the webpage of National Center for Biotechnology Information which can currently be found at http://www.ncbi.nlm.nih.gov/and which was firstly described in Altschul et al. (1990) J. Mol. Biol. 215; 403-410.

In a preferred embodiment of the present invention, the alignment program is performing a global alignment program, which optimizes the alignment over the full-length of the sequences. In a further preferred embodiment, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available at http://www.ebi.ac.uk/Tools/psa/.

EMBOSS Needle performs an optimal global sequence alignment using the Needleman-Wunsch alignment algorithm to find the optimum alignment (including gaps) of two sequences along their entire length.

EMBOSS Stretcher uses a modification of the Needleman-Wunsch algorithm that allows larger sequences to be globally aligned.

In one embodiment, the sequences are aligned by a global alignment program and the sequence identity is calculated by identifying the number of exact matches identified by the program divided by the “alignment length”, where the alignment length is the length of the entire alignment including gaps and overhanging parts of the sequences.

In a further embodiment, the global alignment program uses the Needleman-Wunsch algorithm and the sequence identity is calculated by identifying the number of exact matches identified by the program divided by the “alignment length”, where the alignment length is the length of the entire alignment including gaps and overhanging parts of the sequences.

In yet a further embodiment, the global alignment program is selected from the group consisting of EMBOSS Needle and EMBOSS stretcher and the sequence identity is calculated by identifying the number of exact matches identified by the program divided by the “alignment length”, where the alignment length is the length of the entire alignment including gaps and overhanging parts of the sequences.

Once the software has produced an alignment, it is possible to calculate % similarity and % sequence identity. The software typically does this as part of the sequence comparison and generates a numerical result.

In one embodiment, it is preferred to use the ClustalW software for performing sequence alignments. Preferably, alignment with ClustalW is performed with the following parameters for pairwise alignment:

| | |
|---|---|
| Substitution matrix: | Gonnet 250 |
| Gap open penalty: | 20 |
| Gap extension penalty: | 0.2 |
| Gap end penalty: | None |

ClustalW2 is for example made available on the internet by the European Bioinformatics Institute at the EMBL-EBI webpage www.ebi.ac.uk under tools-sequence analysis-ClustalW2. Currently, the exact address of the ClustalW2 tool is www.ebi.ac.uk/Tools/clustalw2.

In another embodiment, it is preferred to use the program Align X in Vector NTI (Invitrogen) for performing sequence alignments. In one embodiment, Exp10 has been may be used with default settings:

Gap opening penalty: 10
Gap extension penalty: 0.05
Gap separation penalty range: 8

In a another embodiment, the alignment of one amino acid sequence with, or to, another amino acid sequence is determined by the use of the score matrix: blosum62mt2 and the VectorNTI Pair wise alignment settings

| | | |
|---|---|---|
| Settings | K-tuple | 1 |
| | Number of best diagonals | 5 |
| | Window size | 5 |
| | Gap Penalty | 3 |
| | Gap opening Penalty | 10 |
| | Gap extension Penalty | 0.1 |

In one embodiment, the percentage of identity of one amino acid sequence with, or to, another amino acid sequence is determined by the use of Blast with a word size of 3 and with BLOSUM 62 as the substitution matrix

### Polypeptides

In one aspect, disclosed herein is a polypeptide having a ratio of transgalactosylating activity:β-galactosidase activity of at least 0.5, at least 1, at least 2, at least 2.5, at least 3, at least 4, at least 5, at least 6, at least 7, at least 8, at least 9, at least 10, at least 11, or at least 12 at or above a concentration of 3% w/w initial lactose concentration.

In one aspect, disclosed herein is a polypeptide, wherein the glycoside hydrolase catalytic core has an amino acid sequence of SEQ ID NO:7.

In one aspect, disclosed herein is a polypeptide containing a Glyco_hydro2N (PF02837), a Glyco_hydro (PF00703) and/or a Glyco_hydro 2C (PF02836) domains.

In one aspect, disclosed herein is a polypeptide containing the Bacterial Ig-like domain (group 4) (PF07532).

In one aspect, disclosed herein is a polypeptide having transgalactosylating activity selected from the group consisting of:
a. a polypeptide comprising an amino acid sequence having at least 90% sequence identity with SEQ ID NO: 1, wherein said polypeptide consists of at most 980 amino acid residues,
b. a polypeptide comprising an amino acid sequence having at least 97% sequence identity with SEQ ID NO: 2, wherein said polypeptide consists of at most 975 amino acid residues,
c. a polypeptide comprising an amino acid sequence having at least 96.5% sequence identity with SEQ ID NO: 3, wherein said polypeptide consists of at most 1300 amino acid residues,
d. a polypeptide encoded by a polynucleotide that hybridizes under at least low stringency conditions with i) the nucleic acid sequence comprised in SEQ ID NO: 9, 10, 11, 12 or 13 encoding the polypeptide of SEQ ID NO: 1, 2, 3, 4 or 5; or ii) the complementary strand of i),
e. a polypeptide encoded by a polynucleotide comprising a nucleotide sequence having at least 70% identity to the nucleotide sequence encoding for the polypeptide of SEQ ID NO: 1, 2, 3, 4 or 5 or the nucleotide sequence comprised in SEQ ID NO: 9, 10, 11, 12 or 13 encoding a mature polypeptide, and
f. a polypeptide comprising a deletion, insertion and/or conservative substitution of one or more amino acid residues of SEQ ID NO: 1, 2, 3, 4 or 5.

In another aspect disclosed herein is a polypeptide having transgalactosylating activity selected from the group consisting of:
a. a polypeptide comprising an amino acid sequence having at least 96.5% sequence identity with SEQ ID NO: 3, wherein said polypeptide consists of at most 1300 amino acid residues,
b. a polypeptide comprising an amino acid sequence having at least 90% sequence identity with SEQ ID NO: 1, wherein said polypeptide consists of at most 980 amino acid residues,
c. a polypeptide encoded by a polynucleotide that hybridizes under at least low stringency conditions with i) the nucleic acid sequence comprised in SEQ ID NO: 9, 10, 11, 12 or 13 encoding the polypeptide of SEQ ID NO: 1, 2, 3, 4, or 5; or ii) the complementary strand of i),
d. a polypeptide encoded by a polynucleotide comprising a nucleotide sequence having at least 70% identity to the nucleotide sequence encoding for the polypeptide of SEQ ID NO: 1, 2, 3, 4 or 5 or the nucleotide sequence comprised in SEQ ID NO: 9, 10, 11, 12 or 13 encoding a mature polypeptide, and
e. a polypeptide comprising a deletion, insertion and/or conservative substitution of one or more amino acid residues of SEQ ID NO: 1, 2, 3, 4 or 5.

In one aspect, disclosed herein is a polypeptide, wherein the amino acid sequence has at least 68%, 70%, 72%, 74%, 76%, 78%, 80%%, 85%, 90%, 91%, 92%, 93%, 94%, 95%, 96%, 97%, 98%, or 99%, sequence identity to the mature amino acid sequence of SEQ ID NO: 1, 2, 3, 4 or 5.

In one aspect, disclosed herein is a polypeptide having 90% sequence identity to the mature amino acid sequence of SEQ ID NO:1.

In one aspect, disclosed herein is a polypeptide having 90% sequence identity to the mature amino acid sequence of SEQ ID NO:2.

In one aspect, disclosed herein is a polypeptide having 96.5% sequence identity to the mature amino acid sequence of SEQ ID NO:3.

In one aspect, disclosed herein is a polypeptide having 96.5% sequence identity to the mature amino acid sequence of SEQ ID NO:4.

In one aspect, disclosed herein is a polypeptide having 96.5% sequence identity to the mature amino acid sequence of SEQ ID NO:5.

In one aspect, disclosed herein is a polypeptide comprising or consisting of the amino acid sequence of SEQ ID NO:1, 2, 3, 4 or 5.

In one aspect, disclosed herein is a polypeptide, which is derived from Bifidobacterium bifidum.

In one aspect, disclosed herein is a polypeptide having a pH optimum of 6.5-7.5.

In one aspect, disclosed herein is a polypeptide having a temperature optimum of 30-60 such as 42-60° C.

Polypeptides having activity on carbohydrates can be classified using either the IUBMB system of classification based on their substrate specificity or on the CaZy assignment into one of the current 125 glycoside hydrolase family. In the CaZy database the assignment is based on both sequence and structural information combined with knowledge of stereochemistry of the substrates and products

Disclosed herein are polypeptides which when being an expression product in a suitable host strain (e.g., Bacillus subtilis) comprising f a nucleic acid sequence which encodes said polypeptide, is the only polypeptide expression product of said nucleic acid sequence that exhibits transgalactosylating activity. This may be evaluated by using the following techniques know to a person skilled in the art. The samples to be evaluated are subjected to SDS-PAGE and visualized using a dye appropriate for protein quantification, such as for example the Bio-Rad Criterion system. The gel is then scanned using appropriate densiometic scanner such as for example the Bio-Rad Criterion system and the resulting picture is ensured to be in the dynamic range. The bands corresponding to any variant/fragment derived from SEQ ID NO: 8 are quantified and the percentage of the polypeptides are calculated as follows:

Percentage of polypeptide in question=polypeptide in question/(sum of all polypeptides exhibiting transgalactosylating activity)*100.

The total number of polypeptides variants/fragments derived from SEQ ID NO:8 in the composition can be determined by detecting fragment derived from SEQ ID NO:8 by western blotting using a polyclonal antibody by methods know to a person skilled in the art.

The polypeptide disclosed herein comprises at least two separate functional domains contained within the enzyme. Firstly, the polypeptide should contain a glycoside hydrolase catalytic core as described in the following. The catalytic core should belong to the GH-A clan of related glycoside hydrolase families. The GH-A clan is characterized by cleaving glycosidic bonds via a retaining mechanism and possesses a catalytic domain which is based on a TIM barrel fold (Werenga, 2001, FEBS Letters, 492(3), p 193-8). The catalytic domain contains two glutamic acid residues which act as proton donor and nucleophile, eminating from strands 4 and 7 of the barrel domain (Jenkins, 1995, FEBS Letters, 362(3), p 281-5). The overall structure of the TIM barrel is a (β/α) 8 fold consisting of 8 beta strands and 8 alpha-helices. In one aspect, the glycoside hydrolase catalytic core disclosed herein belong to either of the glycoside hydrolase families GH-2, and −35 which are all TIM-barrel enzymes belonging to the GH-A clan. In a further aspect, the glycoside hydrolase catalytic core belong to family GH-2 or GH-35. In a further aspect, the glycoside hydrolase catalytic core belong to family GH-2. A common denominator is that these enzymes are so called retaining enzymes, so that the stereochemistry of the substrate is conserved in the product (Henrissat, 1997, Curr Opin Struct Biol, 7(5), 637-44).

In one aspect, the polypeptides disclosed herein have activity on carbohydrates bonds which has the β(1→4) conformation. This effectively put the enzymes into the IUBMB EC 3.2.1.23 class of β-galactosidases. This activity may be, but is not confined to, determined by utilizing synthetic substrates such as para-nitrophenol-β-D-galactopyranoside (PNPG), ortho-nitrophenol-β-D-galactopyranoside (ONPG) or β-D-galactopyranoside with chromogenic aglycons (XGal). As an alternative way of determining whether an enzyme belong to the EC 3.2.1.23 class of β-galactosidases is to incubate with a substrate such as lactose and measure the release of glucose by a method such as enzymatic determination, HPLC, TLC or other methods known to persons skilled in the art.

In order to predict functional entities of polypeptides several available public repositories can be applied such as for example Pfam (Nucl. Acids Res. (2010) 38 (suppl 1): D211-D222. doi: 10.1093/nar/gkp985) and Interpro (Nucl. Acids Res. (2009) 37 (suppl 1): D211-D215. doi: 10.1093/nar/gkn785). It should be specified that when performing such analysis the analysis should be performed on the full length sequence of the polypeptide available from public repository databases.

In a further aspect, a polypeptide containing one or more Pfam domains selected from: Glyco_hydro2N (PF02837), Glyco_hydro (PF00703), Glyco_hydro 2C (PF02836) and Bacterial Ig-like domain (group 4) (PF07532), is provided. In yet a further aspect, a polypeptide containing the Pfam domains Glyco_hydro2N (PF02837), Glyco_hydro (PF00703), Glyco_hydro 2C (PF02836) and Bacterial Ig-like domain (group 4) (PF07532), is provided. In yet a further aspect, a polypeptide containing the Glyco_hydro2N (PF02837), Glyco_hydro (PF00703), and Glyco_hydro 2C (PF02836) domains which constitutes the catalytic domain of the polypeptide, is provided.

In a further aspect, a polypeptide as disclosed herein and having a ratio of transgalactosylating activity:β-galactosidase activity of at least 1, at least 2.5, at least 3, at least 4, at least 5, at least 6, at least 7, at least 8, at least 9, at least 10, at least 11, or at least 12 as measured at a concentration of 100 ppm in a milk-based assay at 37° C. and 5 w/w % lactose after 15, 30 or 180 such as 180 minutes reaction, is provided. In a further aspect, the polypeptide is derived from Bifidobacterium bifidum.

In one aspect, the herein disclosed polypeptide(s) has a transgalactosylating activity such that more than 20%, more than 30%, more than 40%, up to 50% of the initial lactose is transgalactosylated as measured at a concentration of 100 ppm in a milk-based assay at 37° C. and 5 w/w % lactose after 15, 30 or 180 such as 180 minutes of reaction.

In a further aspect, the herein disclosed polypeptide(s) has a β-galactosidase activity such that less than 80%, less than 70%, less than 60%, less than 50%, less than 40%, less than 30%, less than 20% of the lactose has been hydrolysed as measured at a concentration of 100 ppm in a milk-based assay at 37° C. and 5 w/w % lactose after 15, 30 or 180 such as 180 minutes of reaction.

In one aspect, the β-galactosidase activity and/or the transgalactosylating activity are measured at a concentration of 100 ppm corresponding to 2.13 LAU as specified in Method 4 of WO 2013/182626.

In a further aspect, the herein disclosed polypeptide(s) has one or more of the following characteristics:

a) a ratio of transgalactosylating activity:β-galactosidase activity of at least of at least 1, at least 2.5, at least 3, at least 4, at least 5, at least 6, at least 7, at least 8, at least 9, at least 10, at least 11, or at least 12 as measured at a concentration of 100 ppm in a milk-based assay at 37° C. and 5 w/w % lactose after 15, 30 or 180 such as 180 minutes reaction, and/or
b) has a transgalactosylating activity such that more than 20%, more than 30%, more than 40%, and up to 50% of the initial lactose has been transgalactosylated as measured at a concentration of 100 ppm in a milk-based assay at 37° C. and 5 w/w % lactose after 15, 30 or 180 such as 180 minutes of reaction.

In one aspect, a polypeptide comprising an amino acid sequence having at least 96.5% sequence identity with SEQ ID NO: 3, wherein said polypeptide consists of at most 1300 amino acid residues, is provided. In a further aspect, a polypeptide comprising an amino acid sequence having at least 90% sequence identity with SEQ ID NO: 1 such as wherein said sequence identity is at least 95%, such as, e.g. at least 96%, at least 97%, at least 98%, at least 99% or at least 100% sequence identity, and wherein said polypeptide consists of at most 980 amino acid residues, is provided. In a further aspect, a polypeptide comprising an amino acid sequence having at least 90% sequence identity with SEQ ID NO: 1, wherein said polypeptide consists of at most 980 amino acid residues, is provided. In yet a further aspect, a polypeptide wherein said polypeptide has at least 90% sequence identity with SEQ ID NO: 1, such as wherein said polypeptide has at least 90%, such as, e.g. at least 91%, at least 92%, at least 93%, at least 94%, at least 95%, at least 96%, at least 97%, at least 98%, or at least 99% sequence identity with SEQ ID NO: 1 is provided. In another aspect, a polypeptide having at least 96.5% sequence identity to SEQ ID NO: 2 such as wherein said polypeptide has at least 97%, such as, e.g. at least 98% or at least 99% sequence identity with SEQ ID NO: 2. In one aspect, the polypeptides disclosed herein consist of at the most 975 amino acid residues, such as, e.g. at most 970 amino acid residues, such as at most 950 amino acid residues, such as at most 940 amino acid residues, at most 930 amino acid residues, at most 920 amino acid residues, at most 910 amino acid residues, at most 900 amino acid residues, at most 895 amino acid residues or at most 890 amino acid residues, is provided. In one aspect, a particular polypeptide consists of 887 or 965 amino acid residues, is provided. In one aspect, a polypeptide comprising an amino acid sequence having at least 97% sequence identity with SEQ ID NO: 2 such as wherein said sequence identity is at least 98%, such as, e.g. at least 99% or at least 100% sequence identity, wherein said polypeptide consists of at most 975 amino acid residues, such as, e.g. at most 970 or at least 965 amino acid residues, is provided. In one aspect, a polypeptide comprising an amino acid sequence having at least 97% sequence identity with SEQ ID NO: 2, wherein said polypeptide consists of at most 975 amino acid residues, is provided.

In a further preferred aspect, a polypeptide which comprises SEQ ID NO:1, 2, 3, 4 or 5, is provided. In yet a preferred aspect, a polypeptide consisting of the amino acid sequence of SEQ ID NO: 1, 2, 3, 4, or 5, especially a polypeptide consisting of the amino acid sequence of SEQ ID NO: 1 or 2, is provided.

In a further aspect, a polypeptide comprising an amino acid sequence having at least 96.5% sequence identity with SEQ ID NO: 3 such as wherein said sequence identity is at least 97%, such as, e.g. at least 98%, at least 99% or at least 100% sequence identity, wherein said polypeptide consists of at most 1300 amino acid residues, is provided.

In a further aspect, a polypeptide wherein said polypeptide has at least 98.5%, such as at least 99% or at least 99.5% sequence identity with SEQ ID NO: 5, is provided. In one aspect, such a polypeptide consists of at most 1290 amino acid residues, such as, e.g. at most 1280, at most 1270, at most 1260, at most 1250, at most 1240, at most 1230, at most 1220 or at most 1215 amino acid residues, is provided. In a preferred aspect, a polypeptide which consists of 1211 amino acid residues, is provided.

In a further aspect, a polypeptide wherein said polypeptide has at least 96% such as at least at least 97%, such as, e.g., at least 98% or at least 99% sequence identity with SEQ ID NO: 4, is provided. In one aspect, a polypeptide which consists of at most 1210 amino acid residues, such as, e.g. at most 1200, at most 1190, at most 1180, at most 1170, at most 1160, at most 1150 or at most 1145 amino acid residues, such as 1142 amino acid residues, is provided.

In a further aspect, a polypeptide wherein said polypeptide has at least 96.5% such as at least 97%, such as, e.g., at least 98% or at least 99% sequence identity with SEQ ID NO: 3, is provided. In one aspect, a polypeptide which consists of at most 1130 amino acid residues, such as, e.g. at the most 1120, at the most 1110, at the most 1100, at the most 1090, at the most 1080, at the most 1070, at the most 1060, at the most 1050, at the most 1055 or at the most 1040 amino acid residues, is provided. In a preferred aspect, a polypeptide which consists of 1038 amino acid residues, is provided.

In a further aspect, the polypeptides disclosed herein has a ratio of transgalactosylation activity above 100% such as above 150%, 175% or 200%.

Proteins are generally comprised of one or more functional regions, commonly termed domains. The presence of different domains in varying combinations in different proteins gives rise to the diverse repertoire of proteins found in nature. One way of describing the domains are by the help of the Pfam database which is a large collection of protein domain families as described in “The Pfam protein families database”: R. D. Finn, J. Mistry, J. Tate, P. Coggill, A. Heger, J. E. Pollington, Gavin, P. Gunesekaran, G. Ceric, K. Forslund, L. Holm, E. L. Sonnhammer, S. R. Eddy, A. Bateman Nucleic Acids Research (2010) Database Issue 38:D211-222. Each family is represented by multiple sequence alignments and hidden Markov models (HMMs). In a further aspect, the present inventors have found that the herein provided polypeptide(s) contains one or more of the Pfam domains Glyco_hydro2N (PF02837), Glyco_hydro (PF00703), Glyco_hydro 2C (PF02836) and Bacterial Ig-like domain (group 4) (PF07532). In one aspect, the herein provided polypeptide(s) contains Glyco_hydro2N (PF02837), Glyco_hydro (PF00703), Glyco_hydro 2C (PF02836) and Bacterial Ig-like domain (group 4) (PF07532).

In one aspect, the polypeptides have useful transgalactosylating activity over a range of pH of 4-9, such as 5-8, such as 5.5-7.5, such as 6.5-7.5.

The present invention encompasses polypeptides having a certain degree of sequence identity or sequence homology with amino acid sequence(s) defined herein or with a polypeptide having the specific properties defined herein. The present invention encompasses, in particular, peptides having a degree of sequence identity with any one of SEQ ID NO: 1, 2, 3, 4 or 5, defined below, or homologues thereof.

In one aspect, the homologous amino acid sequence and/or nucleotide sequence should provide and/or encode a polypeptide which retains the functional transgalactosylating activity and/or enhances the transgalactosylating activity compared to a polypeptide of SEQ ID NO: 1, 2, 3, 4 or 5.

In the present context, a homologous sequence is taken to include an amino acid sequence which may be at least 66%, 70%, 75%, 78%, at least 80%, at least 85%, at least 90%, at least 95%, at least 96%, at least 97%, at least 98% or at least 99%, identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

Thus, the present invention also encompasses variants, homologues and derivatives of any amino acid sequence of a protein or polypeptide as defined herein, particularly those of SEQ ID NO: 1, 2, 3, 4 or 5 defined below.

The sequences, particularly those of variants, homologues and derivatives of SEQ ID NO: 1, 2, 3, 4 or 5 defined below, may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The present invention also encompasses conservative substitution (substitution and replacement are both used herein to mean the interchange of an existing amino acid residue, with an alternative residue) that may occur i.e. like-for-like substitution such as basic for basic, acidic for acidic, polar for polar etc. Non-conservative substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyridylalanine, thienylalanine, naphthylalanine and phenylglycine.

Conservative substitutions that may be made are, for example within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxyl amino acids (Serine, Threonine), large amino acids (Phenylalanine and Tryptophan) and small amino acids (Glycine, Alanine).

In one aspect, the polypeptide sequence used in the present invention is in a purified form.

In one aspect, the polypeptide or protein for use in the present invention is in an isolated form.

In one aspect, the polypeptide of the present invention is recombinantly produced.

The variant polypeptides include a polypeptide having a certain percent, e.g., at least 90%, 91%, 92%, 93%, 94%, 95%, 96%, 97%, 98%, or 99%, of sequence identity with SEQ ID NO: 1 or 2.

The variant polypeptides include a polypeptide having a certain percent, e.g., at least 96%, 97%, 98%, or 99%, of sequence identity with SEQ ID NO: 3, 4 or 5.

In one aspect, the polypeptides disclosed herein comprises an amino acid sequence having at least 90%, 91%, 92%, 93%, 94%, 95%, 96%, 97%, 98%, or 99% sequence identity to the amino acid sequence of the mature polypeptide encoded by the nucleotide sequence encoding the transgalactosylase contained in Bifidobacterium bifidum DSM20215 shown herein as SEQ ID NO: 22. All considerations and limitations relating to sequence identities and functionality discussed in terms of the SEQ ID NO: 1, 2, 3, 4 or 5 apply mutatis mutandis to sequence identities and functionality of these polypeptides and nucleotides.

In one aspect, the subject amino acid sequence is SEQ ID NO: 1, 2, 3, 4 or 5, and the subject nucleotide sequence preferably is SEQ ID NO: 9, 10, 11, 12 or 13.

In one aspect, the polypeptide is a fragment having one or more (several) amino acids deleted from the amino and/or carboxyl terminus of the polypeptide of SEQ ID NO: 1, 2, 3, 4 or 5; wherein the fragment has transgalactosylating activity.

In one aspect, a fragment contains at least 500, 550, 600, 650, 700, 750, 800, 850, 900, 950, or 1000 amino acid residues

In a further aspect, the length of the polypeptide variant is 500 to 1300 amino acid residues. In a further aspect, the length of the polypeptide variant is 600 to 1300 amino acids. In a further aspect, the length of the polypeptide variant is 700 to 1300 amino acids. In a further aspect, the length of the polypeptide variant is 800 to 1300 amino acids. In a further aspect, the length of the polypeptide variant is 800 to 1300 amino acids.

### Polypeptide Variants of SEQ ID NO: 1, 2, 3, 4 or 5

In one aspect, a variant of SEQ ID NO: 1, 2, 3, 4 or 5 having a substitution at one or more positions which effects an altered property such as improved transgalactosylation, relative to SEQ ID NO: 1, 2, 3, 4 or 5, is provided. Such variant polypeptides are also referred to in this document for convenience as “variant polypeptide”, “polypeptide variant” or “variant”. In one aspect, the polypeptides as defined herein have an improved transgalactosylating activity as compared to the polypeptide of SEQ ID NO: 1, 2, 3, 4 or 5. In another aspect, the polypeptides as defined herein have an improved reaction velocity as compared to the polypeptide of SEQ ID NO: 1, 2, 3, 4 or 5.

In one aspect, the polypeptides and variants as defined herein exhibit enzyme activity. In one aspect, the polypeptides and the variant polypeptides described herein comprise transgalactosylation activity.

In one aspect, the ratio of transgalactosylating activity:β-galactosidase activity is at least 0.5, such as at least 1, such as at least 1.5, or such as at least 2 after 30 min. reaction such as above a concentration of 3% w/w initial lactose concentration.

In one aspect, the ratio of transgalactosylating activity:β-galactosidase activity is at least 2.5, such as at least 3, such as at least 4, such as at least 5, such as at least 6, such as at least 7, such as at least 8, such as at least 9, such as at least 10, such as at least 11, or such as at least 12 after 30 min. reaction such as above a concentration of 3% w/w initial lactose concentration.

In one aspect, the polypeptides and the variants as defined herein are derivable from microbial sources, in particular from a filamentous fungus or yeast, or from a bacterium. The enzyme may, e.g., be derived from a strain of Agaricus, e.g. A. bisporus; Ascovaginospora; Aspergillus, e.g. A. niger, A. awamori, A. foetidus, A. japonicus, A. oryzae; Candida; Chaetomium; Chaetotomastia; Dictyostelium, e.g. D. discoideum; Kiuveromyces, e.g. K. fragilis, K. lactis; Mucor, e.g. M. javanicus, M. mucedo, M. subtilissimus; Neurospora, e.g. N. crassa; Rhizomucor, e.g. R. pusillus; Rhizopus, e.g. R. arrhizus, R. japonicus, R. stolonifer; Scierotinia, e.g. S. libertiana; Torula; Torulopsis; Trichophyton, e.g. T. rubrum; Whetzelinia, e.g. W. scierotiorum; Bacillus, e.g. B. coagulans, B. circulans, B. megaterium, B. novalis, B. subtilis, B. pumilus, B. stearothermophilus, B. thuringiensis; Bifidobacterium, e.g. B. longum, B. bifidum, B. animalis; Chryseobacterium; Citrobacter, e.g. C. freundii; Clostridium, e.g. C. perfringens; Diplodia, e.g. D. gossypina; Enterobacter, e.g. E. aerogenes, E. cloacae Edwardsiella, E. tarda; Erwinia, e.g. E. herbicola; Escherichia, e.g. E. coli; Klebsiella, e.g. K. pneumoniae; Miriococcum; Myrothesium; Mucor; Neurospora, e.g. N. crassa; Proteus, e.g. P. vulgaris; Providencia, e.g. P. stuartii; Pycnoporus, e.g. Pycnoporus cinnabarinus, Pycnoporus sanguineus; Ruminococcus, e.g. R. torques; Salmonella, e.g. S. typhimurium; Serratia, e.g. S. liquefasciens, S. marcescens; Shigella, e.g. S. flexneri; Streptomyces, e.g. S. antibioticus, S. castaneoglobisporus, S. violeceoruber; Trametes; Trichoderma, e.g. T. reesei, T. viride; Yersinia, e.g. Y. enterocolitica.

An isolated and/or purified polypeptide comprising a polypeptide or a variant polypeptide as defined herein is provided. In one embodiment, the variant polypeptide is a mature form of the polypeptide (SEQ ID NO: 1, 2, 3, 4 or 5). In one aspect, the variants include a C-terminal domain.

In one aspect, a variant polypeptide as defined herein includes variants wherein between one and about 25 amino acid residues have been added or deleted with respect to SEQ ID NO: 1, 2, 3, 4 or 5. In one aspect, a variant polypeptide as defined herein includes variants wherein between one and 25 amino acid residues have been substituted, added or deleted with respect to SEQ ID NO: 1, 2, 3, 4 or 5. In one aspect, the variant has the amino acid sequence of SEQ ID NO: 1, 2, 3, 4 or 5, wherein any number between one and about 25 amino acids have been substituted. In a further aspect, the variant has the amino acid sequence of SEQ ID NO: 1, 2, 3, 4 or 5, wherein any number between three and twelve amino acids has been substituted. In a further aspect, the variant has the amino acid sequence of SEQ ID NO: 1, 2, 3, 4 or 5, wherein any number between five and nine amino acids has been substituted.

In one aspect, at least two, in another aspect at least three, and yet in another aspect at least five amino acids of SEQ ID NO: 1, 2, 3, 4 or 5 have been substituted. In one aspect, the herein disclosed polypeptide(s) has the sequence of 1, 2, 3, 4 or 5. In one aspect, the herein disclosed polypeptide(s) has the sequence of SEQ ID NO: 1, 2, 3, 4 or 5, wherein the 10, such as 9, such as 8, such as 7, such as 6, such 5, such as 4, such as 3, such as 2, such as 1 amino acid in the N-terminal end are substituted and/or deleted.

Enzymes and enzyme variants thereof can be characterized by their nucleic acid and primary polypeptide sequences, by three dimensional structural modeling, and/or by their specific activity. Additional characteristics of the polypeptide or polypeptide variants as defined herein include stability, pH range, oxidation stability, and thermostability, for example. Levels of expression and enzyme activity can be assessed using standard assays known to the artisan skilled in this field. In another aspect, variants demonstrate improved performance characteristics relative to the polypeptide with SEQ ID NO: 1, 2, 3, 4 or 5, such as improved stability at high temperatures, e.g., 65-85° C.

A polypeptide variant is provided as defined herein with an amino acid sequence having at least about 66%, 68%, 70%, 72%, 74%, 78%, 80%, 85%, 90%, 91%, 92%, 93%, 94%, 95%, 96%, 97%, 98%, 99%, 100% identity with the polypeptide of SEQ ID NO: 1, 2, 3, 4 or 5.

### Nucleotides

In one aspect, the present invention relates to isolated polypeptides having transgalactosylating activity as stated above which are encoded by polynucleotides which hybridize under very low stringency conditions, preferably low stringency conditions, more preferably medium stringency conditions, more preferably medium-high stringency conditions, even more preferably high stringency conditions, and most preferably very high stringency conditions with i) the nucleic acid sequence comprised in SEQ ID NO: 9, 10, 11, 12 or 13 encoding the mature polypeptide of SEQ ID NO: 1, 2, 3, 4 or 5; ii) the cDNA sequence of i) or iii) the complementary strand of i) or ii), (J. Sambrook, E. F. Fritsch, and T. Maniatis, 1989, Molecular Cloning, A Laboratory Manual, 2d edition, Cold Spring Harbor, N.Y.). A subsequence of SEQ ID NO: 9, 10, 11, 12 or 13 contains at least 100 contiguous nucleotides or preferably at least 200 continguous nucleotides. Moreover, the subsequence may encode a polypeptide fragment which has lactase activity.

The nucleotide sequence of SEQ ID NO: 9, 10, 11, 12 or 13 or a subsequence thereof, as well as the amino acid sequence of SEQ ID NO: 1, 2, 3, 4 or 5 or a fragment thereof, may be used to design a nucleic acid probe to identify and clone DNA encoding polypeptides having transgalactosylase activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes which are at least 600 nucleotides, at least preferably at least 700 nucleotides, more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA library prepared from such other organisms may, therefore, be screened for DNA which hybridizes with the probes described above and which encodes a polypeptide having lactase activity. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO: 9, 10, 11, 12 or 13 or a subsequence thereof, the carrier material is used in a Southern blot.

For purposes of the present invention, hybridization indicates that the nucleotide sequence hybridizes to a labelled nucleic acid probe corresponding to the nucleotide sequence shown in SEQ ID NO: 9, 10, 11, 12 or 13, its complementary strand, or a subsequence thereof, under very low to very high stringency conditions. Molecules to which the nucleic acid probe hybridizes under these conditions can be detected using X-ray film.

In another preferred aspect, the nucleic acid probe is the mature polypeptide coding region of SEQ ID NO: 9, 10, 11, 12 or 13.

For long probes of at least 100 nucleotides in length, very low to very high stringency conditions are defined as prehybridization and hybridization at 42° C. in 5×SSPE, 0.3% SDS, 200 g/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

In a particular embodiment, the wash is conducted using 0.2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency). In another particular embodiment, the wash is conducted using 0.1×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

Under salt-containing hybridization conditions, the effective Tₘ is what controls the degree of identity required between the probe and the filter bound DNA for successful hybridization. The effective Tₘ may be determined using the formula below to determine the degree of identity required for two DNAs to hybridize under various stringency conditions.

Effective Tₘ=81.5+16.6(log M[Na⁺])+0.41(% G+C)−0.72(% formamide)

(See www.ndsu.nodak.edu/instruct/mcclean/plsc731/dna/dna6.htm)

The G+C content of SEQ ID NO: 10 is 42% and the G+C content of SEQ ID NO: 11 is 44%. For medium stringency, the formamide is 35% and the Na⁺ concentration for 5×SSPE is 0.75 M.

Another relevant relationship is that a 1% mismatch of two DNAs lowers the Tₘ by 1.4° C. To determine the degree of identity required for two DNAs to hybridize under medium stringency conditions at 42° C., the following formula is used:

% Homology=100−[(Effective Tₘ−Hybridization Temperature)/1.4]

(See www.ndsu.nodak.edu/instruct/mcclean/plsc731/dna/dna6.htm)

The variant nucleic acids include a polynucleotide having a certain percent, e.g., 60%, 65%, 70%, 75%, 80%, 85%, 90%, 95%, or 99%, of sequence identity with the nucleic acid encoding SEQ ID NO: 1, 2, 3, 4 or 5. In one aspect, a nucleic acid capable of encoding a polypeptide as disclosed herein, is provided. In a further aspect, the herein disclosed nucleic acid has a nucleic acid sequence which is at least 60%, such as at least 65%, such as at least 70%, such as at least 75%, such as at least 80%, such as at least 85%, such as at least 90%, such as at least 95%, such as at least 99% identical SEQ ID NO: 9, 10, 11, 12 or 13.

In one aspect, a plasmid comprising a nucleic acid as described herein, is provided. In one aspect, an expression vector comprising a nucleic acid as described herein, or capable of expressing a polypeptide as described herein, is provided.

A nucleic acid complementary to a nucleic acid encoding any of the polypeptide variants as defined herein set forth herein is provided. Additionally, a nucleic acid capable of hybridizing to the complement is provided. In another embodiment, the sequence for use in the methods and compositions described here is a synthetic sequence. It includes, but is not limited to, sequences made with optimal codon usage for expression in host organisms, such as yeast.

The polypeptide variants as provided herein may be produced synthetically or through recombinant expression in a host cell, according to procedures well known in the art. In one aspect, the herein disclosed polypeptide(s) is recombinant polypeptide(s). The expressed polypeptide variant as defined herein optionally is isolated prior to use.

In another embodiment, the polypeptide variant as defined herein is purified following expression. Methods of genetic modification and recombinant production of polypeptide variants are described, for example, in U.S. Pat. Nos. 7,371,552, 7,166,453; 6,890,572; and 6,667,065; and U.S. Published Application Nos. 2007/0141693; 2007/0072270; 2007/0020731; 2007/0020727; 2006/0073583; 2006/0019347; 2006/0018997; 2006/0008890; 2006/0008888; and 2005/0137111. The relevant teachings of these disclosures, including polypeptide-encoding polynucleotide sequences, primers, vectors, selection methods, host cells, purification and reconstitution of expressed polypeptide variants, and characterization of polypeptide variants as defined herein, including useful buffers, pH ranges, Ca²⁺ concentrations, substrate concentrations and enzyme concentrations for enzymatic assays, are herein incorporated by reference.

A nucleic acid sequence is provided encoding the protein of SEQ ID NO: 1, 2, 3, 4 or 5 or a nucleic acid sequence having at least about 66%, 68%, 70%, 72%, 74%, 78%, 80%, 85%, 90%, 95%, 96%, 97%, 98%, 99% or 100% sequence identity with a nucleic acid encoding the protein of SEQ ID NO: 1, 2, 3, 4 or 5. In one embodiment, the nucleic acid sequence has at least about 60%, 66%, 68%, 70%, 72%, 74%, 78%, 80%, 85%, 90%, 95%, 96%, 97%, 98% or 99% sequence identity to the nucleic acid of SEQ ID NO: 9, 10, 11, 12 or 13.

### Vectors

In one aspect, the invention relates to a vector comprising a polynucleotide. In one aspect, a bacterial cell comprises the vector. In some embodiments, a DNA construct comprising a nucleic acid encoding a variant is transferred to a host cell in an expression vector that comprises regulatory sequences operably linked to an encoding sequence. The vector may be any vector that can be integrated into a fungal host cell genome and replicated when introduced into the host cell. The FGSC Catalogue of Strains, University of Missouri, lists suitable vectors. Additional examples of suitable expression and/or integration vectors are provided in Sambrook et al., MOLECULAR CLONING: A LABORATORY MANUAL, 3^{rd} ed., Cold Spring Harbor Laboratory Press, Cold Spring Harbor, N.Y. (2001); Bennett et al., MORE GENE MANIPULATIONS IN FUNGI, Academic Press, San Diego (1991), pp. 396-428; and U.S. Pat. No. 5,874,276. Exemplary vectors include pFB6, pBR322, PUC18, pUC100 and pENTR/D, pDON™201, pDONR™221, pENTR™, pGEM®3Z and pGEM®4Z.

Exemplary for use in bacterial cells include pBR322 and pUC19, which permit replication in E. coli, and pE194, for example, which permits replication in Bacillus.

In some embodiments, a nucleic acid encoding a variant is operably linked to a suitable promoter, which allows transcription in the host cell. The promoter may be derived from genes encoding proteins either homologous or heterologous to the host cell. Suitable non-limiting examples of promoters include cbh1, cbh2, egl1, and egl2 promoters. In one embodiment, the promoter is one that is native to the host cell. For example, when P. saccharophila is the host, the promoter is a native P. saccharophila promoter. An “inducible promoter” is a promoter that is active under environmental or developmental regulation. In another embodiment, the promoter is one that is heterologous to the host cell.

In some embodiments, the coding sequence is operably linked to a DNA sequence encoding a signal sequence. In another aspect, a representative signal peptide is SEQ ID NO: 27. A representative signal peptide is SEQ ID NO: 9 which is the native signal sequence of the Bacillus subtilis aprE precursor. In other embodiments, the DNA encoding the signal sequence is replaced with a nucleotide sequence encoding a signal sequence from other extra-cellular Bacillus subtilis pre-cursors. In one embodiment, the polynucleotide that encodes the signal sequence is immediately upstream and in-frame of the polynucleotide that encodes the polypeptide. The signal sequence may be selected from the same species as the host cell.

In additional embodiments, a signal sequence and a promoter sequence comprising a DNA construct or vector to be introduced into a fungal host cell are derived from the same source. In some embodiments, the expression vector also includes a termination sequence. In one embodiment, the termination sequence and the promoter sequence are derived from the same source. In another embodiment, the termination sequence is homologous to the host cell.

In some embodiments, an expression vector includes a selectable marker. Examples of suitable selectable markers include those that confer resistance to antimicrobial agents, e.g., hygromycin or phleomycin. Nutritional selective markers also are suitable and include amdS, argB, and pyr4. In one embodiment, the selective marker is the amdS gene, which encodes the enzyme acetamidase; it allows transformed cells to grow on acetamide as a nitrogen source. The use of an A. nidulans amdS gene as a selective marker is described in Kelley et al., EMBO J. 4: 475-479 (1985) and Penttila et al., Gene 61: 155-164 (1987).

A suitable expression vector comprising a DNA construct with a polynucleotide encoding a variant may be any vector that is capable of replicating autonomously in a given host organism or integrating into the DNA of the host. In some embodiments, the expression vector is a plasmid. In some embodiments, two types of expression vectors for obtaining expression of genes are contemplated. The first expression vector comprises DNA sequences in which the promoter, coding region, and terminator all originate from the gene to be expressed. In some embodiments, gene truncation is obtained by deleting undesired DNA sequences to leave the domain to be expressed under control of its own transcriptional and translational regulatory sequences. The second type of expression vector is preassembled and contains sequences required for high-level transcription and a selectable marker. In some embodiments, the coding region for a gene or part thereof is inserted into this general-purpose expression vector, such that it is under the transcriptional control of the expression construct promoter and terminator sequences. In some embodiments, genes or part thereof are inserted downstream of the strong cbh1 promoter.

### Expression Hosts/Host Cells

In a further aspect, a host cell comprising, preferably transformed with, a plasmid as described herein or an expression vector as described herein, is provided.

In a further aspect, a cell capable of expressing a polypeptide as described herein, is provided.

In one aspect, the host cell as described herein, or the cell as described herein is a bacterial, fungal or yeast cell.

In a further aspect, the host cell is selected from the group consisting of Ruminococcus, Bifidobacterium, Lactococcus, Lactobacillus, Streptococcus, Leuconostoc, Escherichia, Bacillus, Streptomyces, Saccharomyces, Kluyveromyces, Candida, Torula, Torulopsis and Aspergillus.

In a further aspect, the host cell is selected from the group consisting of Ruminococcus hansenii, Bifidobacterium breve, Bifidobacterium longum, Bifidobacterium infantis, Bifidobacterium bifidum and Lactococcus lactis.

In another embodiment, suitable host cells include a Gram positive bacterium selected from the group consisting of Bacillus subtilis, B. licheniformis, B. lentus, B. brevis, B. stearothermophilus, B. alkalophilus, B. amyloliquefaciens, B. coagulans, B. circulans, B. lautus, B. thuringiensis, Streptomyces lividans, or S. murinus; or a Gram negative bacterium, wherein said Gram negative bacterium is Escherichia coli or a Pseudomonas species. In one aspect, the host cell is a B. subtilus or B. licheniformis. In one embodiment, the host cell is B. subtilis, and the expressed protein is engineered to comprise a B. subtilis signal sequence, as set forth in further detail below. In one aspect, the host cell expresses the polynucleotide as set out in the claims.

In some embodiments, a host cell is genetically engineered to express a polypeptide variant as defined herein with an amino acid sequence having at least about 66%, 68%, 70%, 72%, 74%, 78%, 80%, 85%, 90%, 91%, 92%, 93%, 94%, 95%, 96%, 97%, 98%, 99%, 100% identity with the polypeptide of SEQ ID NO: 1, 2, 3, 4 or 5. In some embodiments, the polynucleotide encoding a polypeptide variant as defined herein will have a nucleic acid sequence encoding the protein of SEQ ID NO: 1, 2, 3, 4 or 5 or a nucleic acid sequence having at least about 66%, 68%, 70%, 72%, 74%, 78%, 80%, 85%, 90%, 95%, 96%, 97%, 98%, 99% or 100% sequence identity with a nucleic acid encoding the protein of SEQ ID NO: 1, 2, 3, 4 or 5. In one embodiment, the nucleic acid sequence has at least about 60%, 66%, 68%, 70%, 72%, 74%, 78%, 80%, 85%, 90%, 95%, 96%, 97%, 98% or 99% sequence identity to the nucleic acid of SEQ ID NO: 9, 10, 11, 12 or 13.

### Methods for Producing Polypeptides

In a further aspect, a method of expressing a polypeptide as described herein comprises obtaining a host cell or a cell as described herein and expressing the polypeptide from the cell or host cell, and optionally purifying the polypeptide.

An expression characteristic means an altered level of expression of the variant, when the variant is produced in a particular host cell. Expression generally relates to the amount of active variant that is recoverable from a fermentation broth using standard techniques known in this art over a given amount of time. Expression also can relate to the amount or rate of variant produced within the host cell or secreted by the host cell. Expression also can relate to the rate of translation of the mRNA encoding the variant polypeptide.

### Transformation, Expression and Culture of Host Cells

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Ausubel et al. (1987), supra, chapter 9; Sambrook et al. (2001), supra; and Campbell et al., Curr. Genet. 16: 53-56 (1989). The expression of heterologous protein in Trichoderma is described, for example, in U.S. Pat. Nos. 6,022,725; 6,268,328; Harkki et al., Enzyme Microb. Technol. 13: 227-233 (1991); Harkki et al., BioTechnol. 7: 596-603 (1989); EP 244,234; and EP 215,594. In one embodiment, genetically stable transformants are constructed with vector systems whereby the nucleic acid encoding a variant is stably integrated into a host cell chromosome. Transformants are then purified by known techniques.

In one non-limiting example, stable transformants including an amdS marker are distinguished from unstable transformants by their faster growth rate and the formation of circular colonies with a smooth, rather than ragged outline on solid culture medium containing acetamide. Additionally, in some cases a further test of stability is conducted by growing the transformants on solid non-selective medium, e.g., a medium that lacks acetamide, harvesting spores from this culture medium and determining the percentage of these spores that subsequently germinate and grow on selective medium containing acetamide. Other methods known in the art may be used to select transformants.

### Identification of Activity

To evaluate the expression of a variant in a host cell, assays can measure the expressed protein, corresponding mRNA, or β-galactosidase activity. For example, suitable assays include Northern and Southern blotting, RT-PCR (reverse transcriptase polymerase chain reaction), and in situ hybridization, using an appropriately labeled hybridizing probe. Suitable assays also include measuring activity in a sample. Suitable assays of the activity of the variant include, but are not limited to, ONPG based assays or determining glucose in reaction mixtures such for example described in the methods and examples herein.

### Methods for Purifying Herein Disclosed Polypeptides

In general, a variant produced in cell culture is secreted into the medium and may be purified or isolated, e.g., by removing unwanted components from the cell culture medium. In some cases, a variant may be recovered from a cell lysate. In such cases, the enzyme is purified from the cells in which it was produced using techniques routinely employed by those of skill in the art. Examples include, but are not limited to, affinity chromatography, ion-exchange chromatographic methods, including high resolution ion-exchange, hydrophobic interaction chromatography, two-phase partitioning, ethanol precipitation, reverse phase HPLC, chromatography on silica or on a cation-exchange resin, such as DEAE, chromatofocusing, SDS-PAGE, ammonium sulfate precipitation, and gel filtration using Sephadex G-75, for example. Depending on the intended use the herein disclosed polypeptide(s) may for example be either freeze-dried or prepared in a solution. In one aspect, the herein disclosed polypeptide(s) is freeze-dried form. In another aspect, the herein disclosed polypeptide(s) is in solution.

### Methods for Immobilising and Formulation of the Herein Disclosed Polypeptides

The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the polypeptides or polypeptide compositions used in the method of the invention.

### Methods

In the methods of the present invention, the first saccharide and the second saccharide are contacted with the enzyme to allow the enzyme to transfer the galactose moiety from the first saccharide to the second saccharide. In one embodiment, the enzyme is added to a mixture of the first and second saccharide. In one embodiment, the second saccharide is added to a mixture of the first saccharide and enzyme. In one embodiment, the first saccharide is added to a mixture of the second saccharide and enzyme.

In the methods of the present invention, the first saccharide and the second saccharide are contacted with the enzyme at a temperature such that the enzyme is capable of catalysing the transfer of the galactose moiety from the first saccharide to the second saccharide. The precise temperature depends on factors such as the nature and amount of enzyme and the nature and amounts of the first and second saccharide.

In one embodiment, (in particular, those embodiments where the galactose moiety and the fructose moiety are linked in the final product, such as lactulose) the method is carried out at a temperature of 0 to 100° C. In one embodiment, the method is carried out at a temperature of 0 to 10° C. In one embodiment, the method is carried out at a temperature of 45 to 60° C.

In another embodiment, (in particular, those embodiments where the galactose moiety and the fructose moiety are separated by a moiety other than galactose or fructose in the final product, such as lactosucrose) the method is carried out at a temperature of 0 to 100° C. In one embodiment, the method is carried out at a temperature of 30 to 70° C. In one embodiment, the method is carried out at a temperature of 40 to 60° C., especially 45 to 55° C., and most preferably 50° C.

In the methods of the present invention, the first saccharide and the second saccharide are contacted with the enzyme for a time sufficient to enable the enzyme to catalyse the transfer of the galactose moiety from the first saccharide to the second saccharide. The precise reaction time depends on factors such as the nature and amount of enzyme and the nature and amounts of the first and second saccharide.

In one embodiment, (in particular, those embodiments where the galactose moiety and the fructose moiety are linked in the final product, such as lactulose), the method is carried out for a time of 1 minute to 24 hours. In one embodiment, the method is carried out for a time of 10 minutes to 6 hours. In one embodiment, the method is carried out for a time of 15 minutes to 5 hours.

In another embodiment, (in particular, those embodiments where the galactose moiety and the fructose moiety are separated by a moiety other than galactose or fructose in the final product, such as lactosucrose) the method is carried out for a time of 1 minute to 48 hours. In one embodiment, the method is carried out for a time of 10 minutes to 24 hours. In one embodiment, the method is carried out for a time of 30 minutes to 12 hours, especially 2 to 8 hours.

In the methods of the present invention, the first saccharide and the second saccharide are contacted with the enzyme at a pH typically such that the enzyme is capable of catalysing the transfer of the galactose moiety from the first saccharide to the second saccharide. The precise pH depends on factors such as the nature and amount of enzyme the nature and amounts of the first and second saccharide, and the composition in which the method is carried out.

In one embodiment, (particularly but not exclusively, those embodiments where the method is carried out in situ in a milk composition), the method is carried out at a pH of at least 5.5, such as at least 5.6, such as at least 5.7, such as at least 5.8, such as at least 5.9, such as at least 6.0, such as at least 6.1, such as at least 6.2, such as at least 6.3, such as at least 6.4, such as at least 6.5, such as 5.5 to 9.5, such as 5.75 to 8.5, such as 6.0 to 8.0, such as 6.25 to 7.5, such as 6.4 to 7.0, such as 6.5 to 6.8.

In one embodiment, (in particular, those embodiments where the galactose moiety and the fructose moiety are linked in the final product, such as lactulose), the method is carried out at a pH of at least 5.5, such as at least 5.6, such as at least 5.7, such as at least 5.8, such as at least 5.9, such as at least 6.0, such as at least 6.1, such as at least 6.2, such as at least 6.3, such as at least 6.4, such as at least 6.5, such as 5.5 to 9.5, such as 5.75 to 8.5, such as 6.0 to 8.0, such as 6.25 to 7.5, such as 6.4 to 7.0, such as 6.5 to 6.8.

In another embodiment, (in particular, those embodiments where the galactose moiety and the fructose moiety are separated by a moiety other than galactose or fructose in the final product, such as lactosucrose) the method is carried out at a pH of at least 5.5, such as at least 5.6, such as at least 5.7, such as at least 5.8, such as at least 5.9, such as at least 6.0, such as at least 6.1, such as at least 6.2, such as at least 6.3, such as at least 6.4, such as at least 6.5, such as 5.5 to 9.5, such as 5.75 to 8.5, such as 6.0 to 8.0, such as 6.25 to 7.5, such as 6.4 to 7.0, such as 6.5 to 6.8.

Preferably the combination of temperature, pH and/or the incubation time is effective to ensure that there is at least 5% transferase activity, preferably at least 10% transferase activity, preferably at least 15%, 20%, 25%, 26%, 28%, 30%, 40% 50%, 60% or 75% transferase activity.

In one embodiment, the yield of lactulose is at least 10%. In one embodiment, the yield of lactulose is at least 12%. In one embodiment, the yield of lactulose is at least 15%. In one embodiment, the yield of lactulose is at least 18%. In one embodiment, the yield of lactulose is at least 20%. In one embodiment, the yield of lactulose is at least 22%. In one embodiment, the yield of lactulose is at least 25%. This yield is calculated by weight based on the total weight of lactose and fructose used as starting material.

The methods of the present invention may be carried out in situ in a food composition. In one embodiment, the food composition is a dairy composition. In one embodiment, the food composition is milk or a composition containing milk.

The term ‘milk’ as used herein may comprise milk from either animal or vegetable origin, and includes whole milk, skim milk, and semi-skim milk. It is possible to use milk from animal sources such as buffalo, (traditional) cow, sheep, goat etc. either individually or combined. Vegetable milks such as soya milk may also be used, either alone or in combination with the animal milk. When vegetable milks are used in combination with animal milk, the combination typically comprises a low percentage (of vegetable milk) say below 15%, or below 20%, or below 25% v/v.

In one aspect, disclosed herein is a method for producing a food product by treating a substrate comprising lactose with a polypeptide as described herein.

In one aspect, disclosed herein is a method for producing a dairy product by treating a milk-based substrate comprising lactose with a polypeptide as described herein. In one aspect, the substrate comprising lactose is further treated with a hydrolysing beta-galactosidase.

The enzyme preparation, such as in the form of a food ingredient prepared according to the present invention, may be in the form of a solution or as a solid—depending on the use and/or the mode of application and/or the mode of administration. The solid form can be either as a dried enzyme powder or as a granulated enzyme.

Examples of dry enzyme formulations include spray dried products, mixer granulation products, layered products such as fluid bed granules, extruded or pelletized granules, prilled products, and lyophilized products.

In one aspect, a composition, preferably a food composition, more preferably a dairy product comprising a cell or a polypeptide as described herein, is provided.

In one embodiment, lactose is present as an initial component of the dairy composition. In one embodiment, lactose is added to the dairy composition.

### Applications and Products

The product of the methods of the invention is a saccharide including a galactose moiety and a fructose moiety.

In one embodiment, the product of the methods of the invention is a saccharide in which a galactose moiety is linked to a fructose moiety, typically by a glycosidic bond. In this embodiment, the glycoside bond may be a 1,4′-glycoside bond (which may be a 1,4′-α- or 1,4′-β-glycoside bond), a 1,6′-glycoside bond (which may be a 1,6′-α- or 1,6′-β-glycoside bond), a 1,2′-glycoside bond (which may be a 1,2′-α- or 1,2′β-glycoside bond), or a 1,3′-glycoside bonds (which may be a 1,3′-α- or 1,3′β-glycoside bond). In one embodiment, the glycoside bond is a 1,4′-glycoside bond. In one embodiment, the glycoside bond is a 1,4′-α-glycoside bond. In one embodiment, the glycoside bond is a 1,4′-β-glycoside bond.

In one embodiment, the product is lactulose, i.e. 4-O-β-D-galactopyranosyl-β-D-fructofuranose. This is typically formed by the method of the invention where the first saccharide is lactose and the second saccharide is fructose.

In one embodiment, the product of the methods of the invention is a saccharide in which the galactose moiety and the fructose moiety are separated by at least one monosaccharide moiety other than galactose or fructose. Typically the galactose moiety and the fructose moiety are separated in the product saccharide by from 1 to 10, preferably 1 to 5, more preferably 1, 2 or 3, even more preferably 1 or 2, and most preferably only 1 monosaccharide moiety.

The monosaccharide moiety (or moieties) which separate the galactose moiety and the fructose moiety in the product may be any of the monosaccharide moieties listed above, provided it is not galactose or fructose. In one embodiment, the monosaccharide moiety which separates galactose moiety and the fructose moiety is a glucose moiety.

The monosaccharide moiety (or moieties) which separate the galactose and fructose moieties are typically joined to those moieties by a glycosidic bond. In this embodiment, the glycoside bond may be a 1,4′-glycoside bond (which may be a 1,4′-α- or 1,4′-β-glycoside bond), a 1,6′-glycoside bond (which may be a 1,6′-α- or 1,6′β-glycoside bond), a 1,2′-glycoside bond (which may be a 1,2′-α- or 1,2′-β-glycoside bond), or a 1,3′-glycoside bonds (which may be a 1,3′-α- or 1,3′β-glycoside bond). In one embodiment, the glycoside bond is a 1,4′-glycoside bond. In one embodiment, the glycoside bond is a 1,4′-α-glycoside bond. In one embodiment, the glycoside bond is a 1,4′-β-glycoside bond.

In one embodiment, the product is lactosucrose, i.e. β-D-galactopyranosyl-(1→4)-α-D-glucopyranosyl-(1→2)-β-D-fructofuranose. This is typically formed using the method of the invention where the first saccharide is lactose and the second saccharide is sucrose.

The product (typically lactulose and/or lactosucrose) of the present invention may be incorporated into a foodstuff. The term “foodstuff” as used herein means a substance which is suitable for human and/or animal consumption.

Suitably, the term “foodstuff” as used herein may mean a foodstuff in a form which is ready for consumption. Alternatively or in addition, however, the term foodstuff as used herein may mean one or more food materials which are used in the preparation of a foodstuff. The foodstuff may be in the form of a solution or suspension of emulsion or as a solid—depending on the use and/or the mode of application and/or the mode of administration.

When used as—or in the preparation of—a food—such as functional food—the composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

Examples of foodstuff include, but are not limited to, one or more of the following: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

In certain embodiments, the foodstuff in accordance with the present invention may be a “fine food”, including cakes, pastry, confectionery, chocolates, fudge and the like.

In one aspect the foodstuff in accordance with the present invention may be a dough product or a baked product, such as bread, a fried product, a snack, cakes, pies, brownies, cookies, noodles, snack items such as crackers, graham crackers, pretzels, and potato chips, and pasta.

In a further aspect, the foodstuff in accordance with the present invention may be a plant derived food product such as flours, pre-mixes, oils, fats, cocoa butter, coffee whitener, salad dressings, margarine, spreads, peanut butter, shortenings, ice cream, cooking oils.

In another aspect, the foodstuff in accordance with the present invention may be a dairy product, including butter, milk, cream, cheese such as natural, processed, and imitation cheeses in a variety of forms (including shredded, block, slices or grated), cream cheese, ice cream, frozen desserts, yoghurt, yoghurt drinks, butter fat, anhydrous milk fat, whey-containing foods and drinks, and other dairy products.

The term “milk”, in the context of the present invention, is to be understood as the lacteal secretion obtained from any mammal, such as cows, sheep, goats, buffaloes or camels.

In the present context, the term “milk-based substrate” means any raw and/or processed milk material or a material derived from milk constituents. Useful milk-based substrates include, but are not limited to solutions/suspensions of any milk or milk like products comprising lactose, such as whole or low fat milk, skim milk, buttermilk, reconstituted milk powder, condensed milk, solutions of dried milk, UHT milk, whey, whey permeate, acid whey, or cream. Preferably, the milk-based substrate is milk or an aqueous solution of skim milk powder. The milk-based substrate may be more concentrated than raw milk. In one embodiment, the milk-based substrate has a ratio of protein to lactose of at least 0.2, preferably at least 0.3, at least 0.4, at least 0.5, at least 0.6 or, most preferably, at least 0.7. The milk-based substrate may be homogenized and/or pasteurized according to methods known in the art.

“Homogenizing” as used herein means intensive mixing to obtain a soluble suspension or emulsion. It may be performed so as to break up the milk fat into smaller sizes so that it no longer separates from the milk. This may be accomplished by forcing the milk at high pressure through small orifices.

“Pasteurizing” as used herein means reducing or eliminating the presence of live organisms, such as microorganisms, in the milk-based substrate. Preferably, pasteurization is attained by maintaining a specified temperature for a specified period of time. The specified temperature is usually attained by heating. The temperature and duration may be selected in order to kill or inactivate certain bacteria, such as harmful bacteria, and/or to inactivate enzymes in the milk. A rapid cooling step may follow. A “food product” or “food composition” in the context of the present invention may be any comestible food or feed product suitable for consumption by an animal or human.

A “dairy product” in the context of the present invention may be any food product wherein one of the major constituents is milk-based. Preferable, the major constituent is milk-based. More preferably, the major constituent is a milk-based substrate which has been treated with an enzyme having transgalactosylating activity. A dairy product as described herein may be, e.g., skim milk, low fat milk, whole milk, cream, UHT milk, milk having an extended shelf life, a fermented milk product, cheese, yoghurt, butter, dairy spread, butter milk, acidified milk drink, sour cream, whey based drink, ice cream, condensed milk, dulce de leche or a flavoured milk drink. A dairy product may be manufactured by any method known in the art.

A dairy product may additionally comprise non-milk components, e.g. vegetable components such as, e.g., vegetable oil, vegetable protein, and/or vegetable carbohydrates. Dairy products may also comprise further additives such as, e.g., enzymes, flavouring agents, microbial cultures such as probiotic cultures, salts, sweeteners, sugars, acids, fruit, fruit juices, or any other component known in the art as a component of, or additive to, a dairy product.

In one embodiment of the invention, one or more milk components and/or milk fractions account for at least 50% (weight/weight), such as at least 70%, e.g. at least 80%, preferably at least 90%, of the dairy product.

In one embodiment of the invention, one or more milk-based substrates having been treated with an enzyme as defined herein having transgalactosylating activity account for at least 50% (weight/weight), such as at least 70%, e.g. at least 80%, preferably at least 90%, of the dairy product.

In the present context, “one of the major constituents” means a constituent having a dry matter which constitutes more than 20%, preferably more than 30% or more than 40% of the total dry matter of the dairy product, whereas “the major constituent” means a constituent having a dry matter which constitutes more than 50%, preferably more than 60% or more than 70% of the total dry matter of the dairy product.

A “fermented dairy product” in present context is to be understood as any dairy product wherein any type of fermentation forms part of the production process. Examples of fermented dairy products are products like yoghurt, buttermilk, creme fraiche, quark and fromage frais. Another example of a fermented dairy product is cheese. A fermented dairy product may be produced by any method known in the art.

The term “fermentation” means the conversion of carbohydrates into alcohols or acids through the action of a microorganism such as a starter culture. In one aspect, fermentation comprises conversion of lactose to lactic acid. In this context, “microorganism” may include any bacterium or fungus being able to ferment a milk substrate.

The microorganisms used for most fermented milk products are selected from the group of bacteria generally referred to as lactic acid bacteria. As used herein, the term “lactic acid bacterium” designates a gram-positive, microaerophilic or anaerobic bacterium, which ferments sugars with the production of acids including lactic acid as the predominantly produced acid, acetic acid and propionic acid. The industrially most useful lactic acid bacteria are found within the order Lactobacillales which includes Lactococcus spp., Streptococcus spp., Lactobacillus spp., Leuconostoc spp., Pseudo/euconostoc spp., Pediococcus spp., Brevibacterium spp., Enterococcus spp. and Propionibacterium spp. Additionally, lactic acid producing bacteria belonging to the group of anaerobic bacteria, bifidobacteria, i.e. Bifidobacterium spp., which are frequently used as food cultures alone or in combination with lactic acid bacteria, are generally included in the group of lactic acid bacteria.

Lactic acid bacteria are normally supplied to the dairy industry either as frozen or freeze-dried cultures for bulk starter propagation or as so-called “Direct Vat Set” (DVS) cultures, intended for direct inoculation into a fermentation vessel or vat for the production of a fermented dairy product. Such cultures are in general referred to as “starter cultures” or “starters”.

Commonly used starter culture strains of lactic acid bacteria are generally divided into mesophilic organisms having optimum growth temperatures at about 30° C. and thermophilic organisms having optimum growth temperatures in the range of about 40 to about 45° C. Typical organisms belonging to the mesophilic group include Lactococcus lactis, Lactococcus lactis subsp. cremoris, Leuconostoc mesenteroides subsp. cremoris, Pseudoleuconostoc mesenteroides subsp. cremoris, Pediococcus pentosaceus, Lactococcus lactis subsp. lactis biovar. diacetylactis, Lactobacillus casei subsp. casei and Lactobacillus paracasei subsp. paracasei. Thermophilic lactic acid bacterial species include as examples Streptococcus thermophilus, Enterococcus faecium, Lactobacillus delbrueckii subsp. lactis, Lactobacillus helveticus, Lactobacillus delbrueckii subsp. bulgaricus and Lactobacillus acidophilus. Also the anaerobic bacteria belonging to the genus Bifidobacterium including Bifidobacterium bifidum, Bifidobacterium animalis and Bifidobacterium longum are commonly used as dairy starter cultures and are generally included in the group of lactic acid bacteria. Additionally, species of Propionibacteria are used as dairy starter cultures, in particular in the manufacture of cheese. Additionally, organisms belonging to the Brevibacterium genus are commonly used as food starter cultures.

Another group of microbial starter cultures are fungal cultures, including yeast cultures and cultures of filamentous fungi, which are particularly used in the manufacture of certain types of cheese and beverage. Examples of fungi include Penicillium roqueforti, Penicillium candidum, Geotrichum candidum, Torula kefir, Saccharomyces kefir and Saccharomyces cerevisiae.

In one embodiment of the present invention, the microorganism used for fermentation of the milk-based substrate is Lactobacillus casei or a mixture of Streptococcus thermophilus and Lactobacillus delbrueckii subsp. bulgaricus.

Fermentation processes to be used in a method of the present invention are well known and the person of skill in the art will know how to select suitable process conditions, such as temperature, oxygen, amount and characteristics of microorganism/s, additives such as e.g. carbohydrates, flavours, minerals, enzymes, and process time. Fermentation conditions are selected so as to support the intended product of the present invention.

As a result of fermentation, pH of the milk-based substrate will be lowered. The pH of a fermented dairy product of the invention may be, e.g., in the range 3.5-6, such as in the range 3.5-5, preferably in the range 3.8-4.8.

In another aspect, the foodstuff in accordance with the present invention may be a food product containing animal derived ingredients, such as processed meat products, cooking oils, shortenings.

In a further aspect, the foodstuff in accordance with the present invention may be a beverage, a fruit, mixed fruit, a vegetable, a marinade or wine.

Some foods are nutraceuticals. By “nutraceutical” is meant a food which carries health benefits over and above their nutritional value. Nutraceuticals cross the dividing line between foods and medicine.

The product of the method of the present invention (typically lactulose) may also be incorporated into a pharmaceutical composition. Such compositions may include, in addition to the products of the method of present invention, conventional pharmaceutical excipients, and other conventional, pharmaceutically inactive agents. Additionally, the compositions may include active agents in addition to the product of the method of the present invention.

The compositions may be in liquid, semi-liquid or solid form, formulated in a manner suitable for the route of administration to be used. For oral administration, capsules and tablets are typically used. For parenteral administration, reconstitution of a lyophilized powder, prepared as described herein, is typically used.

Compositions comprising product of the method of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The product of the method of the present invention may also be administered or coadministered in slow release dosage forms.

The product of the method of the present invention may be administered or coadministered in any conventional dosage form. Co-administration in the context of this invention is intended to mean the administration of more than one therapeutic agent, one of which includes a product of the method of the present invention, in the course of a coordinated treatment to achieve an improved clinical outcome. Such co-administration may also be coextensive, that is, occurring during overlapping periods of time.

Solutions or suspensions used for parenteral, intradermal, subcutaneous, or topical application may optionally include one or more of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations may optionally be enclosed in ampules, disposable syringes or single or multiple dose vials made of glass, plastic or other suitable material.

Upon mixing or adding product of the method of the present invention to a composition, a solution, suspension, emulsion or the like may be formed. The form of the resulting composition will depend upon a number of factors, including the intended mode of administration, and the solubility of the compound in the selected carrier or vehicle. The effective concentration needed to ameliorate the disease being treated may be empirically determined.

Compositions according to the present invention are optionally provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the product of the method of the present invention. In addition to one or more compounds according to the present invention, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyvinylpyrrolidine, celluloses and derivatives thereof, povidone, crospovidones and other such binders known to those of skill in the art.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension.

Dosage forms or compositions may optionally comprise one or more products of the method according to the present invention in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium croscarmellose, glucose, sucrose, magnesium carbonate, sodium saccharin, talcum.

Methods for preparing these formulations are known to those skilled in the art. The compositions may optionally contain 0.01%-100% (weight/weight) of one or more products of the method according to the present invention, optionally 0.1-95%, and optionally 1-95%.

### EXAMPLES

### Example 1—Generation of Lactulose

Arla Minimaelk 0.5% fat (Arla, Brabrand, Denmark), containing ˜45 g lactose per litre, was fortified with lactose (Sigma-Aldrich, Schnelldorf, Germany) to concentrations of 45, 70 and 90 g/L lactose. Additionally, fructose (50, 60, 70 and 80 g/L) was added to each particular milk. Subsequently, the β-galactosidase disclosed in Example 4 (also disclosed in WO 2013/182626 as “BIF 917”) (2625 U/L) was added and the milk was incubated at 50° C. for up to 8 hours before the concentration of lactulose was analysed by HPLC.

All standards (lactose, glucose, galactose and GOS) were prepared in double distilled water (ddH₂O) and filtered through 0.45 μm syringe filters. A set of each standard was prepared ranging in concentration from 10 to 200,000 ppm.

To evaluate quantification of the above set of sugars in a yogurt/milk matrix, the above standards were spiked into a milk and yogurt samples and used as internal controls. All milk and yogurt samples containing active β-galactosidase were inactivated by heating the sample to 95° C. for 10 min. All milk samples were prepared in 96 well MTP plates (Corning, N.Y., USA) and diluted minimum 20 times and filtered through 0.20 μm 96 well plate filters before analysis (Corning filter plate, PVDF hydrophile membrane, NY, USA). Samples containing more than 50,000 ppm (5% w/v) lactose were heated to 30° C. to ensure proper solubilization. All yogurt samples were weighted and diluted 10 times in ddH₂O before homogenization of the sample using an Ultra turrax TP18/10 for a few minutes (Janke & Kunkel Ika-Iabortechnik, Bie & Berntsen, Denmark). β-galactosidase were inactivated by heat treatment and samples were further diluted in 96 well MTP plates and filtered through 0.20 μm 96 well plate filters before analysis (Corning filter plate, PVDF hydrophile membrane, NY, USA). All samples were analyzed in 96 well MTP plates sealed with tape.

### Instrumentation

Quantification of galacto-oligosaccharides (GOS), lactose, glucose and galactose were performed by HPLC. Analysis of samples was carried out on a Dionex Ultimate 3000 HPLC system (Thermo Fisher Scientific) equipped with a DGP-3600SD Dual-Gradient analytical pump, WPS-3000TSL thermostated autosampler, TCC-3000SD thermostated column oven, and a RI-101 refractive index detector (Shodex, J M Science). Chromeleon datasystem software (Version 6.80, DU10A Build 2826, 171948) was used for data acquisition and analysis.

### Chromatographic Conditions

The samples were analyzed by HPLC using a RSO oligosaccharide column, Ag⁺4% crosslinked (Phenomenex, The Netherlands) equipped with an analytical guard column (Carbo-Ag⁺ neutral, AJ0-4491, Phenomenex, The Netherlands) operated at 70° C. The column was eluted with double distilled water (filtered through a regenerated cellulose membrane of 0.45 μm and purged with helium gas) at a flow rate of 0.3 ml/min.

Isocratic flow of 0.3 ml/min was maintained throughout analysis with a total run time of 37 min and injection volume was set to 10 μL. Samples were held at 30° C. in the thermostated autosampler compartment to ensure solubilisation of all components. The eluent was monitored by means of a refractive index detector (RI-101, Shodex, J M Science) and quantification was made by the peak area relative to the peak area of the given standard. Peaks with a degree of three or higher (DP3+) in the Vivinal GOS syrup (Friesland Food Domo, The Netherlands) were used as standard for quantification of all galactooligosaccharides (DP3+), following manufactures declaration on the GOS content in the product. The assumption of the same response for all DP3+ galacto-oligosaccharides components was confirmed with mass balances.

Prior to HPLC analysis, the milk was 20-fold diluted in water (95° C./15 min), followed by 0.22 μm filtration.

FIGS. 1, 2 and 3 illustrate the results achieved using 4.5%, 7.0% and 9.0% (w/v) lactose (corresponding to 45, 70 and 90 g/L lactose) respectively. As shown in these Figures, the generated lactulose concentration was dependent on the initially applied lactose and fructose concentration.

Table 1 illustrates the lactulose yield (total sugar) [%] using 7% lactose and various concentrations of fructose.

TABLE 1

| Fructose | Time [h] | | | | | | | |
|---|---|---|---|---|---|---|---|---|
| conc (% w/v) | 0 | 2 | 3 | 4 | 5 | 6 | 7 | 8 |
| 0 hours | 0.0 | 0.0 | 0.0 | 0.0 | 0.0 | 0.0 | 0.0 | 0.0 |
| 5 hours | 0.0 | 12.2 | 18.0 | 21.5 | 24.6 | 24.5 | 25.4 | 23.8 |
| 6 hours | 0.0 | 11.9 | 16.9 | 21.5 | 23.6 | 22.8 | 25.2 | 23.3 |
| 7 hours | 0.0 | 11.1 | 16.7 | 18.2 | 22.1 | 21.7 | 24.1 | 21.9 |
| 8 hours | 0.0 | 10.9 | 15.2 | 17.7 | 20.7 | 21.6 | 22.0 | 20.4 |

### Example 2—Generation of Lactulose

Arla Minimaelk 0.5% fat (Arla, Brabrand, Denmark), containing ˜45 g lactose per litre, was fortified with either fructose (MW: 180.16 Da; Sigma-Aldrich, Schnelldorf, Germany) or ¹³C-labeled fructose (MW: 181.16 Da; Sigma-Aldrich, Schnelldorf, Germany) to 80 g per litre of milk. Subsequently, the β-galactosidase disclosed in Example 4 (also disclosed in WO 2013/182626 as “BIF 917”) (2625 U/L) was added and the milk was incubated at 50° C. for up to 4 hours before the concentration of lactulose was analyzed by HPLC-MS. Prior to HPLC analysis, the milk was 20-fold diluted in water (95° C./15 min). The chromatography was performed on an Agilent 1290 UPLC using a Phenomenex REZEX RSO 4% Ag⁺ doped 200×10 mm ID maintained at 75° C. and eluted with milli-Q water online vacuum degassed at 0.25 ml/min. Injections were 5 μl of samples diluted 10× in water and standards of 4-lactulose, lactose, glucose and galactose (all 100 μg/ml) maintained at 25° C. Samples and standards were centrifuged at 12500×g for 5 minutes prior to use. The column eluate was analyzed in a Bruker Maxis quadrupole time-of-flight mass spectrometer (QTOF MS).

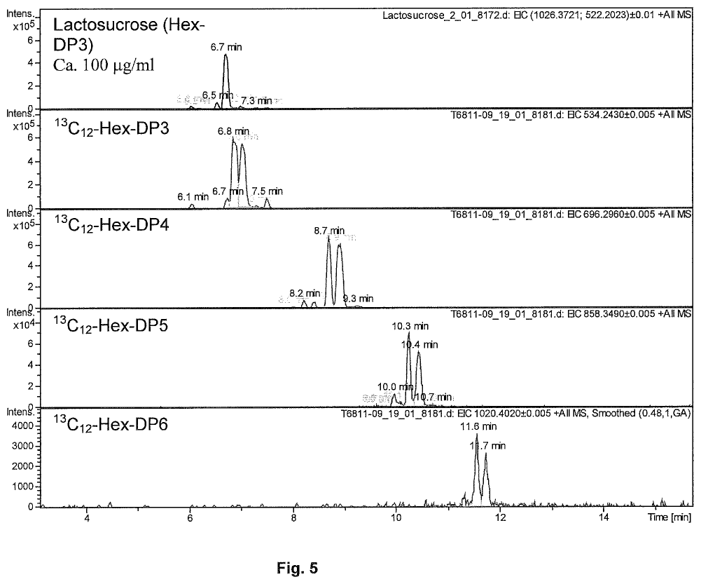

As shown in FIG. 4, both the chromatograms appeared similar independently of the application of non-labeled (MW: 180.16 g/mol) or ¹³C labeled fructose (MW: 181.16 g/mol). The peak eluting at 32.1 min could be assigned as 4-lactulose employing a standard (Sigma-Aldrich, Schnelldorf, Germany). In addition, the detected masses of 366.1086 m/z for the ¹³C labeled fructose (FIG. 4 (A)) and 365.1054 m/z employing the non-labeled fructose (FIG. 4 (B)) at 34.5 min could be assigned to a disaccharide containing fructose, a lactulose isomer, ie a result of a transgalactosylation of the fructose molecule.

### Example 3—Generation of Lactosucrose and Galactosylated Oligomers

### Materials & Methods

Semi-skimmed milk consisting of about 4.6% (w/v) lactose, 3.6% (w/v) protein and 0.5% (w/v) fat (Minimaelk; Arla foods, Viby, Denmark) was supplemented with fully ¹³C labelled sucrose-¹³C₁₂ (Sigma-Aldrich, Schnelldorf, Germany; molecular weight: 354.21 g/mol) or non-labelled sucrose [6% (w/v); Sigma-Aldrich, Schnelldorf, Germany; molecular weight: 342.3 g/mol]. The lactosucrose generation was initiated by addition of the β-galactosidase disclosed in Example 4 (also disclosed in WO 2013/182626 as “BIF 917”). A total activity of 2,625 LAU units was added per litre of milk, corresponding to 1.04 mg enzyme per ml of milk. The lactosucrose generation was performed at 50° C. in an Eppendorf Thermomixer (Eppendorf, Hamburg, Germany) up to 6 hours, samples being taken after 0, 2, 4 and 6 hours. The reaction was terminated by diluting the milk 20-fold in pre-heated water (95 C) and holding for 10 min.

Samples and standards were diluted with four parts of acetonitrile (Sigma-Aldrich, Schnelldorf, Germany) and centrifuged prior to being transferred to injection vials. The detection and analyses of the generated (oligo-galacto)-lactosucrose was conducted with some modifications as described earlier (Hernández-Hernández, Calvillo et al. Journal of Chromatography A, 2012, 1220 57-67.

Diluted solutions were analysed by hydrophilic interaction chromatography using a Waters BEH Amide 2.1×150, 1.7 μm (130 Å) (Waters, Hedehusene, Denmark) column with pre-column and the mobile phases Acetonitrile/water/25% NH₄OH (aqueous) 800/200/1 (v/v/v) (A) and Acetonitrile/water/25% NH₄OH (aqueous) 200/800/1 (v/v/v). Liquid Chromatography was performed on an Agilent 1290 UPLC (Agilent, Waldbronn, Germany) with a flow 350 μl*min⁻¹. The injection volume was 10 μl. The column oven temperature was 35° C. The gradient was as follows 0% B (0 min), 50% B (15 min), cycle time 30 min. The detection was performed with a Bruker maXis QTOF-MS via electrospray positive mode.

The following standards were used (the maltooligoses being used to calibrate the retention time on the column to enable prediction of the retention time of the higher lactosucrose oligomers):

### Maltotriose Batch 017K0679 Opb:T108 Kemikalieskab 5 (Hylde 3)

### Maltotetraose Batch 084K1750 Opb:T108 Kemikalieskab 5 (Hylde 3)

### Maltopentaose Batch 110M1442 Opb:T108 Kemikalieskab 5 (Hylde 3)

### Maltohexaose Batch 048K1472 Opb:T108 Kemikalieskab 5 (Hylde 3)

### Maltoheptaose Batch 029K1194 Opb:T108 Kemikalieskab 5 (Hylde 3)

### Sucrose Batch K29959887 204 Opb:T216

Glucose monohydrate Batch 325 K19714474 Opb:T108 Kemikalieskab 5 (Hylde 2)

### ‘Lactosucrose’ (i.e. 4-O-β-D-galactosylsucrose; Carbosynth) Batch 06448541401 Opb: T216

Stock solutions were combined and diluted to form test solutions with a concentration of each component at ca. 100 μg/ml. A spiked solution was formed by mixing the fully ¹³C labelled sample (sampled after 15-30 min to ensure full dissolution of sucrose) in a 1:1 ratio with a 100 μg/ml standard solution of lactosucrose.

### Results

The extracted ion chromatogram (EIC) of a 100 μg/ml reference sample of the hexose triose (Hex-DP3) lactosucrose (Gal-Glu-Fru) is shown as the upper trace in FIG. 5.

The EIC is based on the exact monoisotopic mass of the adducts of [M+NH₄]⁺ and [2M+NH₄]⁺ with a tolerance of ±0.005 (FIG. 5). In a similar way, the EIC of formed [M+NH₄]⁺ ¹³C₁₂-labelled hexose DP3-6 oligomer (i.e. ¹³C₁₂-Hex-DP3-6) are shown in the traces (FIG. 5). The observed masses correspond to the theoretical mass with a tolerance of ±0.005. Each m/z trace, e.g. for ¹³C₁₂-Hex-DP4, displayed a group of peaks, indicating several isomers. The groups of peaks eluted as expected for a homologous range of carbohydrate oligomers in HILIC with retention time increasing with the number of Hex-DPs. Hence, the chromatograms demonstrate the formation and presence of ¹³C₁₂-Hex DP3-6 oligomers in the labelled sample after 2 hours of biotransformation.

Prior to analysis, the sample was deactivated and 10× diluted. Although the present study is qualitative, the lactosucrose standard solution had a nominal concentration of 100 μg/ml with a peak height of ca. 5×10⁵. The peak height of the ¹³C₁₂-Hex-DP3-4 appeared in the same range, indicating that the concentration of the formed labelled oligomers could be in the range 0.1 mg/ml (injected), and prior to dilution ca. 1 mg/ml in the biotransformation mixture. The ¹³C₁₂-Hex-DP5 and −6 were readily detected, with relative response being 1/10 and 1/100 of the ¹³C₁₂-Hex-DP3-4 levels.

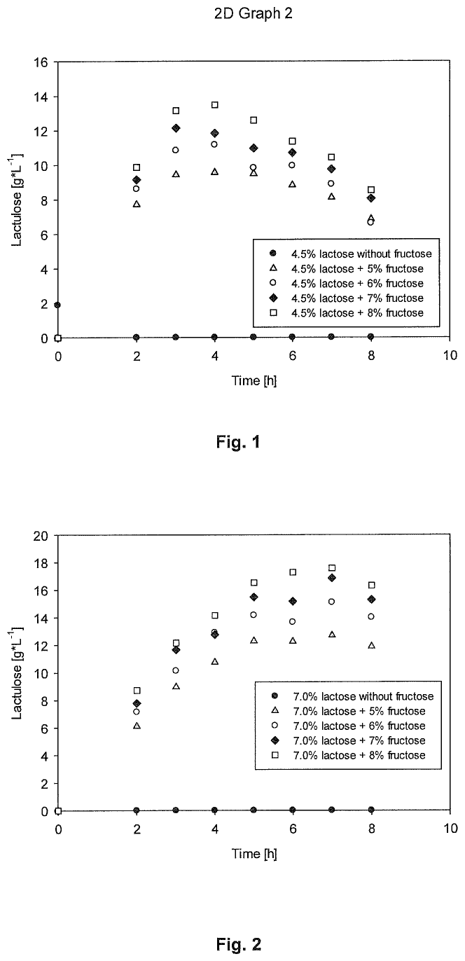

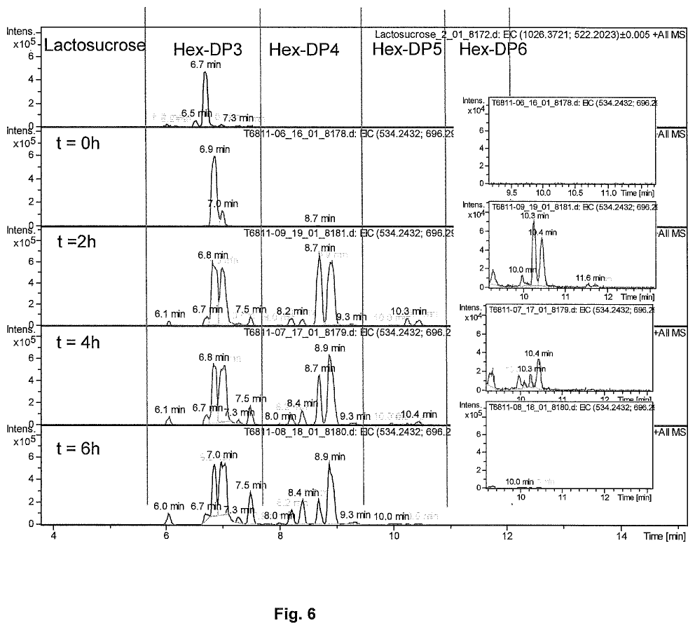

By combining the chromatographic EIC traces outlined in FIG. 2, the relative formation of labelled materials for the biotransformation reaction (t=0, 2, 4, and 6 hours) can be outlined as shown in FIG. 6.

The biotransformation initially resulted in significant amounts of ¹³C₁₂-Hex-DP3 (i.e. fully ¹³C-labelled lactosucrose) already at the first sampling point (t=0), which in practice corresponded to ca. t=0.25-0.5 hour. After 2 hours biotransformation has resulted in larger amounts and more isomers of DP3 ¹³C₁₂-Hex-DP3 but notably significant amounts of higher oligomers. The amount of higher oligomers than ¹³C₁₂-Hex-DP3 oligomers appeared to be reduced after 6 hours of biotransformation, whereas the ¹³C₁₂-Hex-DP3 isomers (especially peak at 6.0; 7.0 and 7.5 min) increased.

### Conclusion

The biotransformation process using ¹³C₁₂-labelled sucrose with the presence resulted in formation of ¹³C₁₂-Hex-DP3-DP6 oligomers, namely Gal-Glu-Fru (i.e. lactosucrose), Gal-Gal-Glu-Fru (i.e. galactosyl-lactosucrose), Gal-Gal-Gal-Glu-Fru (i.e. digalactosyl-lactosucrose) and Gal-Gal-Gal-Gal-Glu-Fru (i.e. trigalactosyl-lactosucrose). The major part of the formed material was ¹³C₁₂-Hex-DP3-4 oligomers. The presence of higher than ¹³C₁₂-Hex-DP3 oligomers increased until 2 hours of reaction and then declined over time.

The experiments therefore confirm that lactosucrose (Galactose-Glucose-Fructose; Gal-Glu-Fru; DP3), galactosyl-lactosucrose (Gal-Gal-Glu-Fru; DP 4) as well as the oligo-galactosyl-lactosucrose (Galₙ-Glu-Fru, whereas n is 2 or 3) have been generated during the transfer reaction of the employed β-galactosidase on sucrose as an acceptor molecule.

### Example 4—Production of Polypeptide and Determination of LAU Activity

### Method 1—Production of Polypeptide

Synthetic genes designed to encode the Bifidobacterium bifidum full length (1752 residues) gene with codons optimised for expression in Bacillus subtilis were purchased from GeneART (Regensburg, Germany) SEQ ID No. 8

The Bifidobacterium bifidum truncation mutants were constructed using polymerase chain reaction with reverse primers that allowed specific amplification of the selected region of the synthetic gene.

Forward primer: GGGGTAACTAGTGGAAGATGCAACAAGAAG (Spel underlined) (SEQ ID NO: 15). The SEQ IDs for the truncation mutants and corresponding reverse primers are indicated in Table 2 below.

TABLE 2

| Truncation | |
|---|---|
| mutant | Primer sequence |
| BIF917 (SEQ | GCGCTTAATTAATTATGTTTTTTCTGTGCTTGTTC |
| ID NO: 9) | SEQ ID NO: 16 |
| BIF995 (SEQ | GCGCTTAATTAATTACAGTGCGCCAATTTCATCAA |
| ID NO: 10) | TCA SEQ ID NO: 17 |
| BIF1068 (SEQ | GCGCTTAATTAATTATTGAACTCTAATTGTCGCTG |
| ID NO: 11) | SEQ ID NO: 18 |
| BIF1172 (SEQ | |
| ID NO: 12) | |
| BIF1241 (SEQ | GCGCTTAATTAATTATGTCGCTGTTTTCAGTTCAA |
| ID NO: 13) | T SEQ ID NO: 19 |
| BIF1326 (SEQ | GCGCTTAATTAATTAAAATTCTTGTTCTGTGCCCA |
| ID NO: 14) | SEQ ID NO: 20 |
| BIF 1478 | GCGCTTAATTAATTATCTCAGTCTAATTTCGCTTG |
| | CGC SEQ ID NO: 21 |

The synthetic gene was cloned into the pBNspe Bacillus subtilis expression vector using the unique restriction sites Spel and Pacl (FIG. 1) and the isolated plasmids were transformed into the Bacillus subtilis strain BG3594. Transformants were restreaked onto LB plates containing 10 μg/mL Neomycin as selection.

A preculture was setup in LB media containing 10 μg/mL Neomycin and cultivated for 7 hours at 37° C. and 180 rpm shaking. 500 μL of this preculture was used to inoculate 50 mL Grant's modified medium containing 10 μg/mL Neomycin at allowed to grow for 68 hours at 33° C. and 180 rpm shaking.

Cells were lysed by addition directly to the culture media of 1 mg/ml Lysozyme (Sigma-Aldrich) and 10 μU/ml Benzonase (Merck) final concentrations and incubated for 1 hr at 33° C. at 180 rpm. Lysates were cleared by centrifugation at 10.000×g for 20 minutes and subsequently sterile filtered.

Grant's modified media was prepared according to the following directions:

PART I (Autoclave)

| | | |
|---|---|---|
| Soytone | 10 | g |

| | |
|---|---|
| Bring to | 500 mL per liter |

PART II

| | | |
|---|---|---|
| 1M K₂HPO₄ | 3 | mL |
| Glucose | 75 | g |
| Urea | 3.6 | g |
| Grant's 10X MOPS | 100 | mL |

Bring to 400 mL per liter

PART I (2 w/w Soytone) was prepared, and autoclaved for 25 minutes at 121° C.
PART II was prepared, and mixed with PART 1 and pH was adjusted to pH to 7.3 with HCl/NaOH.

The volume was brought to full volume and sterilized through 0.22-μm PES filter.

10×MOPS Buffer was prepared according to the following directions:

| | | |
|---|---|---|
| 83.72 | g | Tricine |
| 7.17 | g | KOH Pellets |
| 12 | g | NaCl |
| 29.22 | g | 0.276M K₂SO₄ |
| 10 | mL | 0.528M MgCl₂ |
| 10 | mL | Grant's Micronutrients 100X |

Bring to app. 900 mL with water and dissolve. Adjust pH to 7.4 with KOH, fill up to 1 L and sterile filter the solution through 0.2 μm PES filter.

100×Micronutrients was prepared according to the following directions:

| | |
|---|---|
| Sodium Citrate•2H₂O | 1.47 g |
| CaCl₂•2H₂O | 1.47 g |
| FeSO₄•7H₂O | 0.4 g |
| MnSO₄•H₂O | 0.1 g |
| ZnSO₄•H₂O | 0.1 g |
| CuCl₂•2H₂O | 0.05 g |
| CoCl₂•6H₂O | 0.1 g |
| Na₂MoO₄•2H₂O | 0.1 g |

Dissolve and adjust volume to 1 L with water.
Sterilization was through 0.2 μm PES filter.
Storing was at 4° C. avoid light.

### Method 2—Purification and Enzyme Preparations

The filtrated enzyme isolate was concentrated using a VivaSpin ultra filtration device with a 10 kDa MW cut off (Vivaspin 20, Sartorius, Lot #12VS2004) and the concentrate was loaded onto a PD10 desalting column (GE healthcare, Lot #6284601) and eluted in 20 mM Tris-HCl pH 8.6. Chromatography was carried out manually on an Äkta FPLC system (GE Healthcare). 4 mL of the desalted sample, containing aproximately 20 mg protein, was loaded onto a 2 mL HyperQ column (HyperCel™, Q sorbent) eqillibrated with 20 mM Tris-HCl pH 8.6 at a flowrate of lml/min. The column was thoughroughly washed with 30 CV (column volumes) wash buffer and the bound β-galactosidase was eluted with a 100CV long gradient into 20 mM Tris-HCl pH 8.6 250 mM NaCl. Remaining impurities on the column were removed with a one-step elution using 20 mM Tris-HCl pH 8.6 500 mM NaCl. Protein in the flow through and elution was analyzed for β-galactosidase activity and by SDS-page.

SDS-page gels were run with the Invitrogen NuPage® Novex 4-12% Bis-Tris gel 1.0 mm, 10 well (Cat # NP0321box), See-Blue® Plus2 prestained Standard (Cat # LC5925) and NuPAGE® MES SDS Running Buffer (Cat # NP0002) according to the manufacturer's protocol. Gels were stained with Simply Blue Safestain (Invitrogen, Cat # LC6060) (FIG. 2).

### Method 3—Measuring β-Galactosidase Activity

Enzymatic activity was measured using the commercially available substrate 2-nitrophenyl-β-D-galactopyranoside (ONPG) (Sigma N1127).

### ONPG w/o Acceptor

| | | |
|---|---|---|
| 100 | mM | KₓH₃₋ₓPO₄ (phosphate buffer) at pH 6.0 |
| 12.3 | mM | ONPG |

ONPG Supplemented with Acceptor

| | | |
|---|---|---|
| 100 | mM | KₓH₃₋ₓPO₄ (phosphate buffer) at pH 6.0 |
| 20 | mM | Cellobiose |
| 12.3 | mM | ONPG |

Stop Solution: 10% Na₂Co₃

10 μl dilution series of purified enzyme was added in wells of a microtiter plates containing 90 μl ONPG-buffer with or without acceptor. Samples were mixed and incubated for 10 min at 37° C., subsequently 100 μl STOP Solution were added to each well to terminate reaction. Absorbance measurements were recorded at 420 nm on a Molecular Device SpectraMax platereader controlled by the Softmax software package.

The ratio of transgalactosylation activity was calculated as follows:

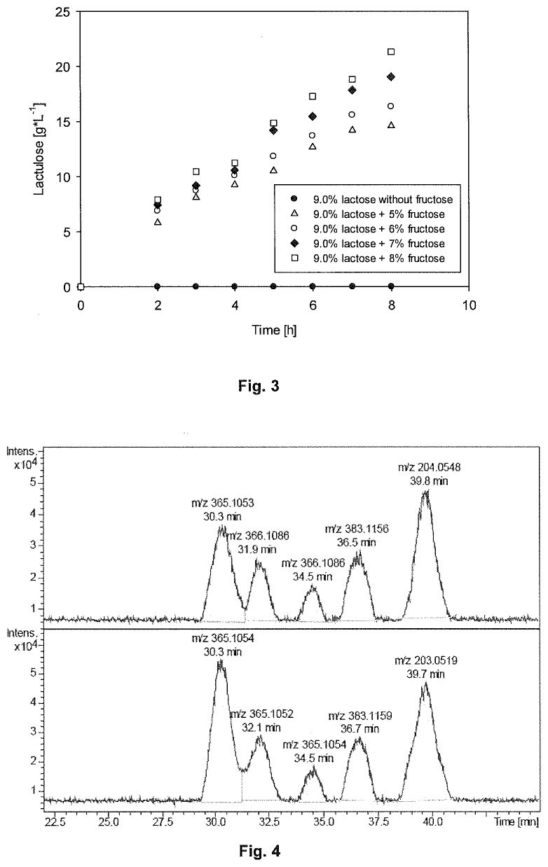

Ratio of transgalctosylation activity=(Abs420^{+Cellobiose}/Abs420^{-Cellobiose})*100, for dilutions where the absorbance was between 0.5 and 1.0 (FIG. 3).

### Method 4—Determination of LAU Activity

### Principle:

The principle of this assay method is that lactase hydrolyzes 2-o-nitrophenyl-β-D-galactopyranoside (ONPG) into 2-o-nitrophenol (ONP) and galactose at 37° C. The reaction is stopped with the sodium carbonate and the liberated ONP is measured in spectrophotometer or colorimeter at 420 nm.

### Reagents:

MES buffer pH 6.4 (100 mM MES pH 6.4, 10 mM CaCl₂): Dissolve 19.52 g MES hydrate (Mw: 195.2 g/mol, Sigma-Aldrich # M8250-250G) and 1.470 g CaCl₂ di-hydrate (Mw: 147.01 g/mol, Sigma-Aldrich) in 1000 ml ddH₂O, adjust pH to 6.4 by 10M NaOH. Filter the solution through 0.2 μm filter and store at 4° C. up to 1 month.
ONPG substrate pH 6.4 (12.28 mM ONPG, 100 mM MES pH 6.4, 10 mM CaCl₂): Dissolve 0.370 g 2-o-nitrophenyl-β-D-galactopyranoside (ONPG, Mw: 301.55 g/mol, Sigma-aldrich # N1127) in 100 ml MES buffer pH 6.4 and store dark at 4° C. for up to 7 days.
Stop reagent (10% Na₂CO₃): Dissolve 20.0 g Na₂CO₃ in 200 ml ddH₂O, Filter the solution through 0.2 μm filter and store at RT up to 1 month.

### Procedure:

Dilution series of the enzyme sample was made in the MES buffer pH 6.4 and 10 μL of each sample dilution were transferred to the wells of a microtiter plate (96 well format) containing 90 μl ONPG substrate pH 6.4. The samples were mixed and incubated for 5 min at 37° C. using a Thermomixer (Comfort Thermomixer, Eppendorf) and subsequently 100 μl Stop reagent was added to each well to terminate the reaction. A blank was constructed using MES buffer pH 6.4 instead of the enzyme sample. The increase in absorbance at 420 nm was measured at a ELISA reader (SpectraMax platereader, Molecular Device) against the blank.

### Calculation of Enzyme Activity:

The molar extinction coefficient of 2-o-nitrophenol (Sigma-Aldrich #33444-25G) in MES buffer pH 6.4 was determined (0.5998×10⁻⁶M⁻¹×cm⁻¹). One unit (U) of lactase activity (LAU) was defined as that corresponding to the hydrolysis of 1 nmol of ONPG per minute. Using microtitre plates with a total reaction volume of 200 μL (light path of 0.52 cm) the lactase activity per mL of the enzyme sample may be calculated using the following equation:

$$LAU \text{/} ml ( \frac{μ mol}{min \cdot mL} ) = \frac{{Abs}_{420} \times 200 μL \times dilution factor}{0 .5998 \cdot {10}^{3} \cdot n {M}^{- 1} \cdot {cm}^{- 1} 10.52 cm \times 5 min \times 0.01 mL}$$

### Calculation of Specific Activity for BIF917 Shown Herein as SEQ ID NO: 1:

Determination of BIF917 concentration:
Quantification of the target enzyme (BIF917) and truncation products were determined using the Criterion Stain free SDS-page system (BioRad). Any kD Stain free precast Gel 4-20% Tris-HCl, 18 well (Comb #345-0418) was used with a Serva Tris-Glycine/SDS buffer (BioRad cat. #42529). Gels were run with the following parameters: 200 V, 120 mA, 25 W, 50 min. BSA (1.43 mg/ml) (Sigma-Aldrich, cat. #500-0007) was used as protein standard and Criterion Stain Free Imager (BioRad) was used with Image Lab software (BioRad) for quantification using band intensity with correlation of the tryptophan content.

The specific LAU activity of BIF917 was determined from crude ferment (ultra filtration concentrate) of two independent fermentations (as described in Method 1) and using 5 different dilutions (see table 3). The specific activity of BIF917 was found to be 21.3 LAU/mg or 0.0213 LAU/ppm.

TABLE 3
Determination of BIF917 specific activity

| | | | | | Protein | Protein | | |
|---|---|---|---|---|---|---|---|---|
| | | | | | (BIF917) | (BIF917) | Specific | Specific |
| Sample | | | Dilution | Activity | concentration | concentration | activity | activity |
| ID | Enzyme | Fermentation | factor | LAU/ml | mg/ml | ppm | LAU/mg | LAU/ppm |
| 1 | BIF 917 | a | 5 | 26.9 | 1.23 | 1232 | 21.9 | 0.0219 |
| 2 | BIF 917 | a | 10 | 53.9 | 2.44 | 2437 | 22.1 | 0.0221 |
| 3 | BIF 917 | a | 10 | 75.4 | 3.56 | 3556 | 21.2 | 0.0212 |
| 4 | BIF 917 | a | 20 | 163.9 | 7.78 | 7778 | 21.1 | 0.0211 |
| 5 | BIF 917 | a | 30 | 233.6 | 11.06 | 11065 | 21.1 | 0.0211 |
| 6 | BIF 917 | b | 5 | 30.26825 | 1.34 | 1342 | 22.6 | 0.0226 |
| 7 | BIF 917 | b | 10 | 55.91536 | 2.61 | 2607 | 21.4 | 0.0214 |
| 8 | BIF 917 | b | 10 | 76.96056 | 3.70 | 3697 | 20.8 | 0.0208 |
| 9 | BIF 917 | b | 20 | 156.986 | 7.75 | 7755 | 20.2 | 0.0202 |
| 10 | BIF 917 | b | 30 | 236.9734 | 11.45 | 11452 | 20.7 | 0.0207 |

| | | |
|---|---|---|
| Arg | 21.3 | 0.0213 |
| Std | 0.700976 | 0.000701 |

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in chemistry, biochemistry, biology, or related fields are intended to be within the scope of the following claims.

List of sequences

SEQ ID NO: 1 (BIF_917)
vedatrsdsttqmsstpevvyssavdskqnrtsdfdanwkfmlsdsvqaqdpafddsawqqvdlphdysitqkys
qsneaesaylpggtgwyrksftidrdlagkriainfdgvymnatvwfngvklgthpygyspfsfdltgnakfggentivv
kvenrlpssrwysgsgiyrdvtltvtdgvhvgnngvaiktpslatqnggdytmnlttkvandteaaanitlkqtvfpkggk
tdaaigtvttasksiaagasadvtstitaaspklwsiknpnlytvrtevlnggkvldtydteygfrwtgfdatsgfslngekv
klkgvsmhhdqgslgavanrraierqveilqkmgvnsirtthnpaakalidvcnekgvlvveevfdmwnrskngnte
dygkwfgqaiagdnavlggdkdetwakfdltstinrdrnapsvimwslgnemmegisgsvsgfpatsaklvawtka
adstrpmtygdnkikanwnesntmgdnltanggvvgtnysdganydkirtthpswaiygsetasainsrgiynrttgg
aqssdkqltsydnsavgwgavassawydvvqrdfvagtyvwtgfdylgeptpwngtgsgavgswpspknsyfgiv
dtagfpkdtyyfyqsqwnddvhtlhilpawnenvvakgsgnnvpvvvytdaakvklyftpkgstekrligeksftkktta
agytyqvyegsdkdstahknmyltwnvpwaegtisaeaydennrlipegstegnasvtttgkaaklkadadrktitad
gkdlsyievdvtdanghivpdaanrvtfdvkgagklvgvdngsspdhdsyqadnrkafsgkvlaivqstkeageitvt
akadglqsstvkiattavpgtstekt
SEQ ID NO: 2 (BIF_995)
vedatrsdsttqmsstpevvyssavdskqnrtsdfdanwkfmlsdsvqaqdpafddsawqqvdlphdysitqkys
qsneaesaylpggtgwyrksftidrdlagkriainfdgvymnatvwfngvklgthpygyspfsfdltgnakfggentivv
kvenrlpssrwysgsgiyrdvtltvtdgvhvgnngvaiktpslatqnggdytmnlttkvandteaaanitlkqtvfpkggk
tdaaigtvttasksiaagasadvtstitaaspklwsiknpnlytvrtevlnggkvldtydteygfrwtgfdatsgfslngekv
klkgvsmhhdqgslgavanrraierqveilqkmgvnsirtthnpaakalidvcnekgvlvveevfdmwnrskngnte
dygkwfgqaiagdnavlggdkdetwakfdltstinrdrnapsvimwslgnemmegisgsvsgfpatsaklvawtka
adstrpmtygdnkikanwnesntmgdnltanggvvgtnysdganydkirtthpswaiygsetasainsrgiynrttgg
aqssdkqltsydnsavgwgavassawydvvqrdfvagtyvwtgfdylgeptpwngtgsgavgswpspknsyfgiv
dtagfpkdtyyfyqsqwnddvhtlhilpawnenvvakgsgnnvpvvvytdaakvklyftpkgstekrligeksftkktta
agytyqvyegsdkdstahknmyltwnvpwaegtisaeaydennrlipegstegnasvtttgkaaklkadadrktitad
gkdlsyievdvtdanghivpdaanrvtfdvkgagklvgvdngsspdhdsyqadnrkafsgkvlaivqstkeageitvt
akadglqsstvkiattavpgtstektvrsfyysrnyyvktgnkpilpsdvevrysdgtsdrqnvtwdavsddqiakagsf
svagtvagqkisvrvtmideigal
SEQ ID NO: 3 (BIF_1068)
Vedatrsdsttqmsstpevvyssavdskqnrtsdfdanwkfmlsdsvqaqdpafddsawqqvdlphdysitqkys
qsneaesaylpggtgwyrksftidrdlagkriainfdgvymnatvwfngvklgthpygyspfsfdltgnakfggentivv
kvenrlpssrwysgsgiyrdvtltvtdgvhvgnngvaiktpslatqnggdvtmnlttkvandteaaanitlkqtvfpkggk
tdaaigtvttasksiaagasadvtstitaaspklwsiknpnlytvrtevlnggkvldtydteygfrwtgfdatsgfslngekv
klkgvsmhhdqgslgavanrraierqveilqkmgvnsirtthnpaakalidvcnekgvlvveevfdmwnrskngnte
dygkwfgqaiagdnavlggdkdetwakfdltstinrdrnapsvimwslgnemmegisgsvsgfpatsaklvawtka
adstrpmtygdnkikanwnesntmgdnltanggvvgtnysdganydkirtthpswaiygsetasainsrgiynrttgg
aqssdkqltsydnsavgwgavassawydvvqrdfvagtyvwtgfdylgeptpwngtgsgavgswpspknsyfgiv
dtagfpkdtyyfyqsqwnddvhtlhilpawnenvvakgsgnnvpvvvytdaakvklyftpkgstekrligeksftkktta
agytyqvyegsdkdstahknmyltwnvpwaegtisaeaydennrlipegstegnasvtttgkaaklkadadrktitad
gkdlsyievdvtdanghivpdaanrvtfdvkgagklvgvdngsspdhdsyqadnrkafsgkvlaivqstkeageitvt
akadglqsstvkiattavpgtstektvrsfyysrnyyvktgnkpilpsdvevrysdgtsdrqnvtwdaysddqiakagsf
svagtvagqkisvrytmideigallnysastpvgtpavlpgsrpavlpdgtvtsanfavhwtkpadtvyntagtvkvpgt
atvfgkefkvtatirvq
SEQ ID NO: 4 (BIF_1172)
vedatrsdsttqmsstpevvyssavdskqnrtsdfdanwkfmlsdsvqaqdpafddsawqqvdlphdysitqkys
qsneaesaylpggtgwyrksftidrdlagkriainfdgvymnatvwfngvklgthpygyspfsfdltgnakfggentivv
kvenrlpssrwysgsgiyrdvtltvtdgvhvgnngvaiktpslatqnggdytmnlttkvandteaaanitlkqtvfpkggk
tdaaigtvttasksiaagasadvtstitaaspklwsiknpnlytvrtevlnggkvldtydteygfrwtgfdatsgfslngekv
klkgvsmhhdqgslgavanrraierqveilqkmgvnsirtthnpaakalidvcnekgvlvveevfdmwnrskngnte
dygkwfgqaiagdnavlggdkdetwakfdltstinrdrnapsvimwslgnemmegisgsvsgfpatsaklvawtka
adstrpmtygdnkikanwnesntmgdnltanggvvgtnysdganydkirtthpswaiygsetasainsrgiynrttgg
aqssdkqltsydnsavgwgavassawydvvqrdfvagtyvwtgfdylgeptpwngtgsgavgswpspknsyfgiv
dtagfpkdtyyfyqsqwnddvhtlhilpawnenvvakgsgnnvpvvvytdaakvklyftpkgstekrligeksftkktta
agytyqvyegsdkdstahknmyltwnvpwaegtisaeaydennrlipegstegnasytttgkaaklkadadrktitad
gkdlsyievdvtdanghivpdaanrvtfdvkgagklvgvdngsspdhdsyqadnrkafsgkvlaivqstkeageitvt
akadglqsstvkiattavpgtstektvrsfyysrnyyvktgnkpilpsdvevrysdgtsdrqnvtwdaysddqiakagsf
svagtvagqkisvrvtmideigallnysastpvgtpavlpgsrpavlpdgtvtsanfavhwtkpadtvyntagtvkvpgt
atvfgkefkvtatirvqrsqvtigssysgnalrltqnipadkqsdtldaikdgsttvdantggganpsawtnwayskagh
ntaeitfeyateqqlgqivmyffrdsnavrfpdagktkiqi
SEQ ID NO: 5 (BIF_1241)
vedatrsdsttqmsstpevvyssavdskqnrtsdfdanwkfmlsdsvqaqdpafddsawqqvdlphdysitqkys
qsneaesaylpggtgwyrksftidrdlagkriainfdgvymnatvwfngvklgthpygyspfsfdltgnakfggentivv
kvenrlpssrwysgsgiyrdvtltvtdgvhvgnngvaiktpslatqnggdvtmnlttkvandteaaanitlkqtvfpkggk
tdaaigtvttasksiaagasadvtstitaaspklwsiknpnlytvrtevlnggkvldtydteygfrwtgfdatsgfslngekv
klkgvsmhhdqgslgavanrraierqveilqkmgvnsirtthnpaakalidvcnekgvlvveevfdmwnrskngnte
dygkwfgqaiagdnavlggdkdetwakfdltstinrdrnapsvimwslgnemmegisgsvsgfpatsaklvawtka
adstrpmtygdnkikanwnesntmgdnltanggvvgtnysdganydkirtthpswaiygsetasainsrgiynrttgg
aqssdkqltsydnsavgwgavassawydvvqrdfvagtyvwtgfdylgeptpwngtgsgavgswpspknsyfgiv
dtagfpkdtyyfyqsqwnddvhtlhilpawnenvvakgsgnnvpvvvytdaakvklyftpkgstekrligeksftkktta
agytyqvyegsdkdstahknmyltwnvpwaegtisaeaydennrlipegstegnasvtttgkaaklkadadrktitad
gkdlsyievdvtdanghivpdaanrvtfdvkgagklvgvdngsspdhdsyqadnrkafsgkvlaivqstkeageitvt
akadglqsstvkiattavpgtstektvrsfyysrnyyvktgnkpilpsdvevrysdgtsdrqnvtwdaysddqiakagsf
svagtvagqkisvrvtmideigallnysastpvgtpavlpgsrpavlpdgtvtsanfavhwtkpadtvyntagtvkvpgt
atvfgkefkvtatirvqrsqvtigssysgnalrltqnipadkqsdtldaikdgsttvdantggganpsawtnwayskagh
ntaeitfeyateqqlgqivmyffrdsnavrfpdagktkiqisadgknwtdlaatetiaaqessdrvkpytydfapvgatfv
kvtvtnadtttpsgvvcaglteielktat
SEQ ID NO: 6 (BIF_1326)
vedatrsdsttqmsstpevvyssavdskqnrtsdfdanwkfmlsdsvqaqdpafddsawqqvdlphdysitqkys
qsneaesaylpggtgwyrksftidrdlagkriainfdgvymnatvwfngvklgthpygyspfsfdltgnakfggentivv
kvenrlpssrwysgsgiyrdvtltvtdgvhvgnngvaiktpslatqnggdvtmnlttkvandteaaanitlkqtvfpkggk
tdaaigtvttasksiaagasadvtstitaaspklwsiknpnlytvrtevlnggkvldtydteygfrwtgfdatsgfslngekv
klkgvsmhhdqgslgavanrraierqveilqkmgvnsirtthnpaakalidvcnekgvlvveevfdmwnrskngnte
dygkwfgqaiagdnavlggdkdetwakfdltstinrdrnapsvimwslgnemmegisgsysgfpatsaklvawtka
adstrpmtygdnkikanwnesntmgdnltanggvvgtnysdganydkirtthpswaiygsetasainsrgiynrttgg
aqssdkqltsydnsavgwgavassawydvvqrdfvagtyvwtgfdylgeptpwngtgsgavgswpspknsyfgiv
dtagfpkdtyyfyqsqwnddvhtlhilpawnenvvakgsgnnvpvvvytdaakvklyftpkgstekrligeksftkktta
agytyqvyegsdkdstahknmyltwnvpwaegtisaeaydennrlipegstegnasvtttgkaaklkadadrktitad
gkdlsyievdvtdanghivpdaanrvtfdvkgagklvgvdngsspdhdsyqadnrkafsgkvlaivqstkeageitvt
akadglqsstvkiattavpgtstektvrsfyysrnyyvktgnkpilpsdvevrysdgtsdrqnvtwdaysddqiakagsf
svagtvagqkisvrvtmideigallnysastpvgtpavlpgsrpavlpdgtvtsanfavhwtkpadtvyntagtvkvpgt
atvfgkefkvtatirvqrsqvtigssysgnalrltqnipadkqsdtldaikdgsttvdantggganpsawtnwayskagh
ntaeitfeyateqqlgqivmyffrdsnavrfpdagktkiqisadgknwtdlaatetiaaqessdrvkpytydfapvgatfv
kvtvtnadtttpsgvvcaglteielktatskfvtntsaalssltvngtkvsdsvlaagsyntpaiiadvkaegegnasvtvlp
andnvirvitesedhvtrktftinlgteqef
SEQ ID NO: 7 Bifidobacterium bifidum glycoside hydrolase catalytic core
qnrtsdfdanwkfmlsdsvqaqdpafddsawqqvdlphdysitqkysqsneaesaylpggtgwyrksftidrdlagk
riainfdgvymnatvwfngvklgthpygyspfsfdltgnakfggentivvkvenrlpssrwysgsgiyrdvtltvtdgvhv
gnngvaiktpslatqnggdytmnlttkvandteaaanitlkqtvfpkggktdaaigtvttasksiaagasadvtstitaasp
klwsiknpnlytvrtevlnggkvldtydteygfrwtgfdatsgfslngekvklkgvsmhhdqgslgavanrraierqveilq
kmgvnsirtthnpaakalidvcnekgvlvveevfdmwnrskngntedygkwfgqaiagdnavlggdkdetwakfdl
tstinrdrnapsvimwslgnemmegisgsysgfpatsaklvawtkaadstrpmty
SEQ ID NO: 8 nucleotide sequence encoding full length
gcagttgaagatgcaacaagaagcgatagcacaacacaaatgtcatcaacaccggaagttgtttattcatcagcggt
cgatagcaaacaaaatcgcacaagcgattttgatgcgaactggaaatttatgctgtcagatagcgttcaagcacaag
atccggcatttgatgattcagcatggcaacaagttgatctgccgcatgattatagcatcacacagaaatatagccaaag
caatgaagcagaatcagcatatcttccgggaggcacaggctggtatagaaaaagctttacaattgatagagatctgg
caggcaaacgcattgcgattaattttgatggcgtctatatgaatgcaacagtctggtttaatggcgttaaactgggcaca
catccgtatggctattcaccgttttcatttgatctgacaggcaatgcaaaatttggcggagaaaacacaattgtcgtcaaa
gttgaaaatagactgccgtcatcaagatggtattcaggcagcggcatttatagagatgttacactgacagttacagatg
gcgttcatgttggcaataatggcgtcgcaattaaaacaccgtcactggcaacacaaaatggcggagatgtcacaatg
aacctgacaacaaaagtcgcgaatgatacagaagcagcagcgaacattacactgaaacagacagtttttccgaaa
ggcggaaaaacggatgcagcaattggcacagttacaacagcatcaaaatcaattgcagcaggcgcatcagcagat
gttacaagcacaattacagcagcaagcccgaaactgtggtcaattaaaaacccgaacctgtatacagttagaacag
aagttctgaacggaggcaaagttctggatacatatgatacagaatatggctttcgctggacaggctttgatgcaacatc
aggcttttcactgaatggcgaaaaagtcaaactgaaaggcgttagcatgcatcatgatcaaggctcacttggcgcagtt
gcaaatagacgcgcaattgaaagacaagtcgaaatcctgcaaaaaatgggcgtcaatagcattcgcacaacacat
aatccggcagcaaaagcactgattgatgtctgcaatgaaaaaggcgttctggttgtcgaagaagtctttgatatgtgga
accgcagcaaaaatggcaacacggaagattatggcaaatggtttggccaagcaattgcaggcgataatgcagttct
gggaggcgataaagatgaaacatgggcgaaatttgatcttacatcaacaattaaccgcgatagaaatgcaccgtca
gttattatgtggtcactgggcaatgaaatgatggaaggcatttcaggctcagtttcaggctttccggcaacatcagcaaa
actggttgcatggacaaaagcagcagattcaacaagaccgatgacatatggcgataacaaaattaaagcgaactg
gaacgaatcaaatacaatgggcgataatctgacagcaaatggcggagttgttggcacaaattattcagatggcgcaa
actatgataaaattcgtacaacacatccgtcatgggcaatttatggctcagaaacagcatcagcgattaatagccgtgg
catttataatagaacaacaggcggagcacaatcatcagataaacagctgacaagctatgataattcagcagttggct
ggggagcagttgcatcatcagcatggtatgatgttgttcagagagattttgtcgcaggcacatatgtttggacaggatttg
attatctgggcgaaccgacaccgtggaatggcacaggctcaggcgcagttggctcatggccgtcaccgaaaaatag
ctattttggcatcgttgatacagcaggctttccgaaagatacatattatttttatcagagccagtggaatgatgatgttcata
cactgcatattcttccggcatggaatgaaaatgttgttgcaaaaggctcaggcaataatgttccggttgtcgtttatacag
atgcagcgaaagtgaaactgtattttacaccgaaaggctcaacagaaaaaagactgatcggcgaaaaatcatttac
aaaaaaaacaacagcggcaggctatacatatcaagtctatgaaggcagcgataaagattcaacagcgcataaaa
acatgtatctgacatggaatgttccgtgggcagaaggcacaatttcagcggaagcgtatgatgaaaataatcgcctga
ttccggaaggcagcacagaaggcaacgcatcagttacaacaacaggcaaagcagcaaaactgaaagcagatgc
ggatcgcaaaacaattacagcggatggcaaagatctgtcatatattgaagtcgatgtcacagatgcaaatggccatat
tgttccggatgcagcaaatagagtcacatttgatgttaaaggcgcaggcaaactggttggcgttgataatggctcatca
ccggatcatgattcatatcaagcggataaccgcaaagcattttcaggcaaagtcctggcaattgttcagtcaacaaaa
gaagcaggcgaaattacagttacagcaaaagcagatggcctgcaatcaagcacagttaaaattgcaacaacagca
gttccgggaacaagcacagaaaaaacagtccgcagcttttattacagccgcaactattatgtcaaaacaggcaaca
aaccgattctgccgtcagatgttgaagttcgctattcagatggaacaagcgatagacaaaacgttacatgggatgcag
tttcagatgatcaaattgcaaaagcaggctcattttcagttgcaggcacagttgcaggccaaaaaattagcgttcgcgtc
acaatgattgatgaaattggcgcactgctgaattattcagcaagcacaccggttggcacaccggcagttcttccgggat
caagaccggcagtcctgccggatggcacagtcacatcagcaaattttgcagtccattggacaaaaccggcagatac
agtctataatacagcaggcacagtcaaagtaccgggaacagcaacagtttttggcaaagaatttaaagtcacagcg
acaattagagttcaaagaagccaagttacaattggctcatcagtttcaggaaatgcactgagactgacacaaaatatt
ccggcagataaacaatcagatacactggatgcgattaaagatggctcaacaacagttgatgcaaatacaggcgga
ggcgcaaatccgtcagcatggacaaattgggcatattcaaaagcaggccataacacagcggaaattacatttgaata
tgcgacagaacaacaactgggccagatcgtcatgtatttttttcgcgatagcaatgcagttagatttccggatgctggca
aaacaaaaattcagatcagcgcagatggcaaaaattggacagatctggcagcaacagaaacaattgcagcgcaa
gaatcaagcgatagagtcaaaccgtatacatatgattttgcaccggttggcgcaacatttgttaaagtgacagtcacaa
acgcagatacaacaacaccgtcaggcgttgtttgcgcaggcctgacagaaattgaactgaaaacagcgacaagca
aatttgtcacaaatacatcagcagcactgtcatcacttacagtcaatggcacaaaagtttcagattcagttctggcagca
ggctcatataacacaccggcaattatcgcagatgttaaagcggaaggcgaaggcaatgcaagcgttacagtccttcc
ggcacatgataatgttattcgcgtcattacagaaagcgaagatcatgtcacacgcaaaacatttacaatcaacctggg
cacagaacaagaatttccggctgattcagatgaaagagattatccggcagcagatatgacagtcacagttggctcag
aacaaacatcaggcacagcaacagaaggaccgaaaaaatttgcagtcgatggcaacacatcaacatattggcata
gcaattggacaccgacaacagttaatgatctgtggatcgcgtttgaactgcaaaaaccgacaaaactggatgcactg
agatatcttccgcgtccggcaggctcaaaaaatggcagcgtcacagaatataaagttcaggtgtcagatgatggaac
aaactggacagatgcaggctcaggcacatggacaacggattatggctggaaactggcggaatttaatcaaccggtc
acaacaaaacatgttagactgaaagcggttcatacatatgcagatagcggcaacgataaatttatgagcgcaagcg
aaattagactgagaaaagcggtcgatacaacggatatttcaggcgcaacagttacagttccggcaaaactgacagtt
gatagagttgatgcagatcatccggcaacatttgcaacaaaagatgtcacagttacactgggagatgcaacactgag
atatggcgttgattatctgctggattatgcaggcaatacagcagttggcaaagcaacagtgacagttagaggcattgat
aaatattcaggcacagtcgcgaaaacatttacaattgaactgaaaaatgcaccggcaccggaaccgacactgacat
cagttagcgtcaaaacaaaaccgagcaaactgacatatgttgtcggagatgcatttgatccggcaggcctggttctgc
aacatgatagacaagcagatagacctccgcaaccgctggttggcgaacaagcggatgaacgcggactgacatgc
ggcacaagatgcgatagagttgaacaactgcgcaaacatgaaaatagagaagcgcatagaacaggcctggatca
tctggaatttgttggcgcagcagatggcgcagttggagaacaagcaacatttaaagtccatgtccatgcagatcaggg
agatggcagacatgatgatgcagatgaacgcgatattgatccgcatgttccggtcgatcatgcagttggcgaactggc
aagagcagcatgccatcatgttattggcctgagagtcgatacacatagacttaaagcaagcggctttcaaattccggct
gatgatatggcagaaatcgatcgcattacaggctttcatcgttttgaacgccatgtc
SEQ ID NO: 9 nucleotide sequence encoding BIF_917
gttgaagatgcaacaagaagcgatagcacaacacaaatgtcatcaacaccggaagttgtttattcatcagcggtcgat
agcaaacaaaatcgcacaagcgattttgatgcgaactggaaatttatgctgtcagatagcgttcaagcacaagatccg
gcatttgatgattcagcatggcaacaagttgatctgccgcatgattatagcatcacacagaaatatagccaaagcaatg
aagcagaatcagcatatcttccgggaggcacaggctggtatagaaaaagctttacaattgatagagatctggcaggc
aaacgcattgcgattaattttgatggcgtctatatgaatgcaacagtctggtttaatggcgttaaactgggcacacatccg
tatggctattcaccgttttcatttgatctgacaggcaatgcaaaatttggcggagaaaacacaattgtcgtcaaagttgaa
aatagactgccgtcatcaagatggtattcaggcagcggcatttatagagatgttacactgacagttacagatggcgttc
atgttggcaataatggcgtcgcaattaaaacaccgtcactggcaacacaaaatggcggagatgtcacaatgaacctg
acaacaaaagtcgcgaatgatacagaagcagcagcgaacattacactgaaacagacagtttttccgaaaggcgga
aaaacggatgcagcaattggcacagttacaacagcatcaaaatcaattgcagcaggcgcatcagcagatgttacaa
gcacaattacagcagcaagcccgaaactgtggtcaattaaaaacccgaacctgtatacagttagaacagaagttctg
aacggaggcaaagttctggatacatatgatacagaatatggctttcgctggacaggctttgatgcaacatcaggcttttc
actgaatggcgaaaaagtcaaactgaaaggcgttagcatgcatcatgatcaaggctcacttggcgcagttgcaaata
gacgcgcaattgaaagacaagtcgaaatcctgcaaaaaatgggcgtcaatagcattcgcacaacacataatccgg
cagcaaaagcactgattgatgtctgcaatgaaaaaggcgttctggttgtcgaagaagtctttgatatgtggaaccgcag
caaaaatggcaacacggaagattatggcaaatggtttggccaagcaattgcaggcgataatgcagttctgggaggc
gataaagatgaaacatgggcgaaatttgatcttacatcaacaattaaccgcgatagaaatgcaccgtcagttattatgt
ggtcactgggcaatgaaatgatggaaggcatttcaggctcagtttcaggctttccggcaacatcagcaaaactggttgc
atggacaaaagcagcagattcaacaagaccgatgacatatggcgataacaaaattaaagcgaactggaacgaat
caaatacaatgggcgataatctgacagcaaatggcggagttgttggcacaaattattcagatggcgcaaactatgata
aaattcgtacaacacatccgtcatgggcaatttatggctcagaaacagcatcagcgattaatagccgtggcatttataat
agaacaacaggcggagcacaatcatcagataaacagctgacaagctatgataattcagcagttggctggggagca
gttgcatcatcagcatggtatgatgttgttcagagagattttgtcgcaggcacatatgtttggacaggatttgattatctggg
cgaaccgacaccgtggaatggcacaggctcaggcgcagttggctcatggccgtcaccgaaaaatagctattttggca
tcgttgatacagcaggctttccgaaagatacatattatttttatcagagccagtggaatgatgatgttcatacactgcatatt
cttccggcatggaatgaaaatgttgttgcaaaaggctcaggcaataatgttccggttgtcgtttatacagatgcagcgaa
agtgaaactgtattttacaccgaaaggctcaacagaaaaaagactgatcggcgaaaaatcatttacaaaaaaaaca
acagcggcaggctatacatatcaagtctatgaaggcagcgataaagattcaacagcgcataaaaacatgtatctgac
atggaatgttccgtgggcagaaggcacaatttcagcggaagcgtatgatgaaaataatcgcctgattccggaaggca
gcacagaaggcaacgcatcagttacaacaacaggcaaagcagcaaaactgaaagcagatgcggatcgcaaaa
caattacagcggatggcaaagatctgtcatatattgaagtcgatgtcacagatgcaaatggccatattgttccggatgc
agcaaatagagtcacatttgatgttaaaggcgcaggcaaactggttggcgttgataatggctcatcaccggatcatgat
tcatatcaagcggataaccgcaaagcattttcaggcaaagtcctggcaattgttcagtcaacaaaagaagcaggcga
aattacagttacagcaaaagcagatggcctgcaatcaagcacagttaaaattgcaacaacagcagttccgggaaca
agcacagaaaaaaca
SEQ ID NO: 10 nucleotide sequence encoding BIF_995
gttgaagatgcaacaagaagcgatagcacaacacaaatgtcatcaacaccggaagttgtttattcatcagcggtcgat
agcaaacaaaatcgcacaagcgattttgatgcgaactggaaatttatgctgtcagatagcgttcaagcacaagatccg
gcatttgatgattcagcatggcaacaagttgatctgccgcatgattatagcatcacacagaaatatagccaaagcaatg
aagcagaatcagcatatcttccgggaggcacaggctggtatagaaaaagctttacaattgatagagatctggcaggc
aaacgcattgcgattaattttgatggcgtctatatgaatgcaacagtctggtttaatggcgttaaactgggcacacatccg
tatggctattcaccgttttcatttgatctgacaggcaatgcaaaatttggcggagaaaacacaattgtcgtcaaagttgaa
aatagactgccgtcatcaagatggtattcaggcagcggcatttatagagatgttacactgacagttacagatggcgttc
atgttggcaataatggcgtcgcaattaaaacaccgtcactggcaacacaaaatggcggagatgtcacaatgaacctg
acaacaaaagtcgcgaatgatacagaagcagcagcgaacattacactgaaacagacagtttttccgaaaggcgga
aaaacggatgcagcaattggcacagttacaacagcatcaaaatcaattgcagcaggcgcatcagcagatgttacaa
gcacaattacagcagcaagcccgaaactgtggtcaattaaaaacccgaacctgtatacagttagaacagaagttctg
aacggaggcaaagttctggatacatatgatacagaatatggctttcgctggacaggctttgatgcaacatcaggcttttc
actgaatggcgaaaaagtcaaactgaaaggcgttagcatgcatcatgatcaaggctcacttggcgcagttgcaaata
gacgcgcaattgaaagacaagtcgaaatcctgcaaaaaatgggcgtcaatagcattcgcacaacacataatccgg
cagcaaaagcactgattgatgtctgcaatgaaaaaggcgttctggttgtcgaagaagtctttgatatgtggaaccgcag
caaaaatggcaacacggaagattatggcaaatggtttggccaagcaattgcaggcgataatgcagttctgggaggc
gataaagatgaaacatgggcgaaatttgatcttacatcaacaattaaccgcgatagaaatgcaccgtcagttattatgt
ggtcactgggcaatgaaatgatggaaggcatttcaggctcagtttcaggctttccggcaacatcagcaaaactggttgc
atggacaaaagcagcagattcaacaagaccgatgacatatggcgataacaaaattaaagcgaactggaacgaat
caaatacaatgggcgataatctgacagcaaatggcggagttgttggcacaaattattcagatggcgcaaactatgata
aaattcgtacaacacatccgtcatgggcaatttatggctcagaaacagcatcagcgattaatagccgtggcatttataat
agaacaacaggcggagcacaatcatcagataaacagctgacaagctatgataattcagcagttggctggggagca
gttgcatcatcagcatggtatgatgttgttcagagagattttgtcgcaggcacatatgtttggacaggatttgattatctggg
cgaaccgacaccgtggaatggcacaggctcaggcgcagttggctcatggccgtcaccgaaaaatagctattttggca
tcgttgatacagcaggctttccgaaagatacatattatttttatcagagccagtggaatgatgatgttcatacactgcatatt
cttccggcatggaatgaaaatgttgttgcaaaaggctcaggcaataatgttccggttgtcgtttatacagatgcagcgaa
agtgaaactgtattttacaccgaaaggctcaacagaaaaaagactgatcggcgaaaaatcatttacaaaaaaaaca
acagcggcaggctatacatatcaagtctatgaaggcagcgataaagattcaacagcgcataaaaacatgtatctgac
atggaatgttccgtgggcagaaggcacaatttcagcggaagcgtatgatgaaaataatcgcctgattccggaaggca
gcacagaaggcaacgcatcagttacaacaacaggcaaagcagcaaaactgaaagcagatgcggatcgcaaaa
caattacagcggatggcaaagatctgtcatatattgaagtcgatgtcacagatgcaaatggccatattgttccggatgc
agcaaatagagtcacatttgatgttaaaggcgcaggcaaactggttggcgttgataatggctcatcaccggatcatgat
tcatatcaagcggataaccgcaaagcattttcaggcaaagtcctggcaattgttcagtcaacaaaagaagcaggcga
aattacagttacagcaaaagcagatggcctgcaatcaagcacagttaaaattgcaacaacagcagttccgggaaca
agcacagaaaaaacagtccgcagcttttattacagccgcaactattatgtcaaaacaggcaacaaaccgattctgcc
gtcagatgttgaagttcgctattcagatggaacaagcgatagacaaaacgttacatgggatgcagtttcagatgatcaa
attgcaaaagcaggctcattttcagttgcaggcacagttgcaggccaaaaaattagcgttcgcgtcacaatgattgatg
aaattggcgcactg
SEQ ID NO: 11 nucleotide sequence encoding BIF_1068
gttgaagatgcaacaagaagcgatagcacaacacaaatgtcatcaacaccggaagttgtttattcatcagcggtcgat
agcaaacaaaatcgcacaagcgattttgatgcgaactggaaatttatgctgtcagatagcgttcaagcacaagatccg
gcatttgatgattcagcatggcaacaagttgatctgccgcatgattatagcatcacacagaaatatagccaaagcaatg
aagcagaatcagcatatcttccgggaggcacaggctggtatagaaaaagctttacaattgatagagatctggcaggc
aaacgcattgcgattaattttgatggcgtctatatgaatgcaacagtctggtttaatggcgttaaactgggcacacatccg
tatggctattcaccgttttcatttgatctgacaggcaatgcaaaatttggcggagaaaacacaattgtcgtcaaagttgaa
aatagactgccgtcatcaagatggtattcaggcagcggcatttatagagatgttacactgacagttacagatggcgttc
atgttggcaataatggcgtcgcaattaaaacaccgtcactggcaacacaaaatggcggagatgtcacaatgaacctg
acaacaaaagtcgcgaatgatacagaagcagcagcgaacattacactgaaacagacagtttttccgaaaggcgga
aaaacggatgcagcaattggcacagttacaacagcatcaaaatcaattgcagcaggcgcatcagcagatgttacaa
gcacaattacagcagcaagcccgaaactgtggtcaattaaaaacccgaacctgtatacagttagaacagaagttctg
aacggaggcaaagttctggatacatatgatacagaatatggctttcgctggacaggctttgatgcaacatcaggcttttc
actgaatggcgaaaaagtcaaactgaaaggcgttagcatgcatcatgatcaaggctcacttggcgcagttgcaaata
gacgcgcaattgaaagacaagtcgaaatcctgcaaaaaatgggcgtcaatagcattcgcacaacacataatccgg
cagcaaaagcactgattgatgtctgcaatgaaaaaggcgttctggttgtcgaagaagtctttgatatgtggaaccgcag
caaaaatggcaacacggaagattatggcaaatggtttggccaagcaattgcaggcgataatgcagttctgggaggc
gataaagatgaaacatgggcgaaatttgatcttacatcaacaattaaccgcgatagaaatgcaccgtcagttattatgt
ggtcactgggcaatgaaatgatggaaggcatttcaggctcagtttcaggctttccggcaacatcagcaaaactggttgc
atggacaaaagcagcagattcaacaagaccgatgacatatggcgataacaaaattaaagcgaactggaacgaat
caaatacaatgggcgataatctgacagcaaatggcggagttgttggcacaaattattcagatggcgcaaactatgata
aaattcgtacaacacatccgtcatgggcaatttatggctcagaaacagcatcagcgattaatagccgtggcatttataat
agaacaacaggcggagcacaatcatcagataaacagctgacaagctatgataattcagcagttggctggggagca
gttgcatcatcagcatggtatgatgttgttcagagagattttgtcgcaggcacatatgtttggacaggatttgattatctggg
cgaaccgacaccgtggaatggcacaggctcaggcgcagttggctcatggccgtcaccgaaaaatagctattttggca
tcgttgatacagcaggctttccgaaagatacatattatttttatcagagccagtggaatgatgatgttcatacactgcatatt
cttccggcatggaatgaaaatgttgttgcaaaaggctcaggcaataatgttccggttgtcgtttatacagatgcagcgaa
agtgaaactgtattttacaccgaaaggctcaacagaaaaaagactgatcggcgaaaaatcatttacaaaaaaaaca
acagcggcaggctatacatatcaagtctatgaaggcagcgataaagattcaacagcgcataaaaacatgtatctgac
atggaatgttccgtgggcagaaggcacaatttcagcggaagcgtatgatgaaaataatcgcctgattccggaaggca
gcacagaaggcaacgcatcagttacaacaacaggcaaagcagcaaaactgaaagcagatgcggatcgcaaaa
caattacagcggatggcaaagatctgtcatatattgaagtcgatgtcacagatgcaaatggccatattgttccggatgc
agcaaatagagtcacatttgatgttaaaggcgcaggcaaactggttggcgttgataatggctcatcaccggatcatgat
tcatatcaagcggataaccgcaaagcattttcaggcaaagtcctggcaattgttcagtcaacaaaagaagcaggcga
aattacagttacagcaaaagcagatggcctgcaatcaagcacagttaaaattgcaacaacagcagttccgggaaca
agcacagaaaaaacagtccgcagcttttattacagccgcaactattatgtcaaaacaggcaacaaaccgattctgcc
gtcagatgttgaagttcgctattcagatggaacaagcgatagacaaaacgttacatgggatgcagtttcagatgatcaa
attgcaaaagcaggctcattttcagttgcaggcacagttgcaggccaaaaaattagcgttcgcgtcacaatgattgatg
aaattggcgcactgctgaattattcagcaagcacaccggttggcacaccggcagttcttccgggatcaagaccggca
gtcctgccggatggcacagtcacatcagcaaattttgcagtccattggacaaaaccggcagatacagtctataataca
gcaggcacagtcaaagtaccgggaacagcaacagtttttggcaaagaatttaaagtcacagcgacaattagagttc
aa
SEQ ID NO: 12 nucleotide sequence encoding BIF_1172
gttgaagatgcaacaagaagcgatagcacaacacaaatgtcatcaacaccggaagttgtttattcatcagcggtcgat
agcaaacaaaatcgcacaagcgattttgatgcgaactggaaatttatgctgtcagatagcgttcaagcacaagatccg
gcatttgatgattcagcatggcaacaagttgatctgccgcatgattatagcatcacacagaaatatagccaaagcaatg
aagcagaatcagcatatcttccgggaggcacaggctggtatagaaaaagctttacaattgatagagatctggcaggc
aaacgcattgcgattaattttgatggcgtctatatgaatgcaacagtctggtttaatggcgttaaactgggcacacatccg
tatggctattcaccgttttcatttgatctgacaggcaatgcaaaatttggcggagaaaacacaattgtcgtcaaagttgaa
aatagactgccgtcatcaagatggtattcaggcagcggcatttatagagatgttacactgacagttacagatggcgttc
atgttggcaataatggcgtcgcaattaaaacaccgtcactggcaacacaaaatggcggagatgtcacaatgaacctg
acaacaaaagtcgcgaatgatacagaagcagcagcgaacattacactgaaacagacagtttttccgaaaggcgga
aaaacggatgcagcaattggcacagttacaacagcatcaaaatcaattgcagcaggcgcatcagcagatgttacaa
gcacaattacagcagcaagcccgaaactgtggtcaattaaaaacccgaacctgtatacagttagaacagaagttctg
aacggaggcaaagttctggatacatatgatacagaatatggctttcgctggacaggctttgatgcaacatcaggcttttc
actgaatggcgaaaaagtcaaactgaaaggcgttagcatgcatcatgatcaaggctcacttggcgcagttgcaaata
gacgcgcaattgaaagacaagtcgaaatcctgcaaaaaatgggcgtcaatagcattcgcacaacacataatccgg
cagcaaaagcactgattgatgtctgcaatgaaaaaggcgttctggttgtcgaagaagtctttgatatgtggaaccgcag
caaaaatggcaacacggaagattatggcaaatggtttggccaagcaattgcaggcgataatgcagttctgggaggc
gataaagatgaaacatgggcgaaatttgatcttacatcaacaattaaccgcgatagaaatgcaccgtcagttattatgt
ggtcactgggcaatgaaatgatggaaggcatttcaggctcagtttcaggctttccggcaacatcagcaaaactggttgc
atggacaaaagcagcagattcaacaagaccgatgacatatggcgataacaaaattaaagcgaactggaacgaat
caaatacaatgggcgataatctgacagcaaatggcggagttgttggcacaaattattcagatggcgcaaactatgata
aaattcgtacaacacatccgtcatgggcaatttatggctcagaaacagcatcagcgattaatagccgtggcatttataat
agaacaacaggcggagcacaatcatcagataaacagctgacaagctatgataattcagcagttggctggggagca
gttgcatcatcagcatggtatgatgttgttcagagagattttgtcgcaggcacatatgtttggacaggatttgattatctggg
cgaaccgacaccgtggaatggcacaggctcaggcgcagttggctcatggccgtcaccgaaaaatagctattttggca
tcgttgatacagcaggctttccgaaagatacatattatttttatcagagccagtggaatgatgatgttcatacactgcatatt
cttccggcatggaatgaaaatgttgttgcaaaaggctcaggcaataatgttccggttgtcgtttatacagatgcagcgaa
agtgaaactgtattttacaccgaaaggctcaacagaaaaaagactgatcggcgaaaaatcatttacaaaaaaaaca
acagcggcaggctatacatatcaagtctatgaaggcagcgataaagattcaacagcgcataaaaacatgtatctgac
atggaatgttccgtgggcagaaggcacaatttcagcggaagcgtatgatgaaaataatcgcctgattccggaaggca
gcacagaaggcaacgcatcagttacaacaacaggcaaagcagcaaaactgaaagcagatgcggatcgcaaaa
caattacagcggatggcaaagatctgtcatatattgaagtcgatgtcacagatgcaaatggccatattgttccggatgc
agcaaatagagtcacatttgatgttaaaggcgcaggcaaactggttggcgttgataatggctcatcaccggatcatgat
tcatatcaagcggataaccgcaaagcattttcaggcaaagtcctggcaattgttcagtcaacaaaagaagcaggcga
aattacagttacagcaaaagcagatggcctgcaatcaagcacagttaaaattgcaacaacagcagttccgggaaca
agcacagaaaaaacagtccgcagcttttattacagccgcaactattatgtcaaaacaggcaacaaaccgattctgcc
gtcagatgttgaagttcgctattcagatggaacaagcgatagacaaaacgttacatgggatgcagtttcagatgatcaa
attgcaaaagcaggctcattttcagttgcaggcacagttgcaggccaaaaaattagcgttcgcgtcacaatgattgatg
aaattggcgcactgctgaattattcagcaagcacaccggttggcacaccggcagttcttccgggatcaagaccggca
gtcctgccggatggcacagtcacatcagcaaattttgcagtccattggacaaaaccggcagatacagtctataataca
gcaggcacagtcaaagtaccgggaacagcaacagtttttggcaaagaatttaaagtcacagcgacaattagagttc
aaagaagccaagttacaattggctcatcagtttcaggaaatgcactgagactgacacaaaatattccggcagataaa
caatcagatacactggatgcgattaaagatggctcaacaacagttgatgcaaatacaggcggaggcgcaaatccgt
cagcatggacaaattgggcatattcaaaagcaggccataacacagcggaaattacatttgaatatgcgacagaaca
acaactgggccagatcgtcatgtatttttttcgcgatagcaatgcagttagatttccggatgctggcaaaacaaaaattca
gatc
SEQ ID NO: 13 nucleotide sequence encoding BIF_1241
gttgaagatgcaacaagaagcgatagcacaacacaaatgtcatcaacaccggaagttgtttattcatcagcggtcgat
agcaaacaaaatcgcacaagcgattttgatgcgaactggaaatttatgctgtcagatagcgttcaagcacaagatccg
gcatttgatgattcagcatggcaacaagttgatctgccgcatgattatagcatcacacagaaatatagccaaagcaatg
aagcagaatcagcatatcttccgggaggcacaggctggtatagaaaaagctttacaattgatagagatctggcaggc
aaacgcattgcgattaattttgatggcgtctatatgaatgcaacagtctggtttaatggcgttaaactgggcacacatccg
tatggctattcaccgttttcatttgatctgacaggcaatgcaaaatttggcggagaaaacacaattgtcgtcaaagttgaa
aatagactgccgtcatcaagatggtattcaggcagcggcatttatagagatgttacactgacagttacagatggcgttc
atgttggcaataatggcgtcgcaattaaaacaccgtcactggcaacacaaaatggcggagatgtcacaatgaacctg
acaacaaaagtcgcgaatgatacagaagcagcagcgaacattacactgaaacagacagtttttccgaaaggcgga
aaaacggatgcagcaattggcacagttacaacagcatcaaaatcaattgcagcaggcgcatcagcagatgttacaa
gcacaattacagcagcaagcccgaaactgtggtcaattaaaaacccgaacctgtatacagttagaacagaagttctg
aacggaggcaaagttctggatacatatgatacagaatatggctttcgctggacaggctttgatgcaacatcaggcttttc
actgaatggcgaaaaagtcaaactgaaaggcgttagcatgcatcatgatcaaggctcacttggcgcagttgcaaata
gacgcgcaattgaaagacaagtcgaaatcctgcaaaaaatgggcgtcaatagcattcgcacaacacataatccgg
cagcaaaagcactgattgatgtctgcaatgaaaaaggcgttctggttgtcgaagaagtctttgatatgtggaaccgcag
caaaaatggcaacacggaagattatggcaaatggtttggccaagcaattgcaggcgataatgcagttctgggaggc
gataaagatgaaacatgggcgaaatttgatcttacatcaacaattaaccgcgatagaaatgcaccgtcagttattatgt
ggtcactgggcaatgaaatgatggaaggcatttcaggctcagtttcaggctttccggcaacatcagcaaaactggttgc
atggacaaaagcagcagattcaacaagaccgatgacatatggcgataacaaaattaaagcgaactggaacgaat
caaatacaatgggcgataatctgacagcaaatggcggagttgttggcacaaattattcagatggcgcaaactatgata
aaattcgtacaacacatccgtcatgggcaatttatggctcagaaacagcatcagcgattaatagccgtggcatttataat
agaacaacaggcggagcacaatcatcagataaacagctgacaagctatgataattcagcagttggctggggagca
gttgcatcatcagcatggtatgatgttgttcagagagattttgtcgcaggcacatatgtttggacaggatttgattatctggg
cgaaccgacaccgtggaatggcacaggctcaggcgcagttggctcatggccgtcaccgaaaaatagctattttggca
tcgttgatacagcaggctttccgaaagatacatattatttttatcagagccagtggaatgatgatgttcatacactgcatatt
cttccggcatggaatgaaaatgttgttgcaaaaggctcaggcaataatgttccggttgtcgtttatacagatgcagcgaa
agtgaaactgtattttacaccgaaaggctcaacagaaaaaagactgatcggcgaaaaatcatttacaaaaaaaaca
acagcggcaggctatacatatcaagtctatgaaggcagcgataaagattcaacagcgcataaaaacatgtatctgac
atggaatgttccgtgggcagaaggcacaatttcagcggaagcgtatgatgaaaataatcgcctgattccggaaggca
gcacagaaggcaacgcatcagttacaacaacaggcaaagcagcaaaactgaaagcagatgcggatcgcaaaa
caattacagcggatggcaaagatctgtcatatattgaagtcgatgtcacagatgcaaatggccatattgttccggatgc
agcaaatagagtcacatttgatgttaaaggcgcaggcaaactggttggcgttgataatggctcatcaccggatcatgat
tcatatcaagcggataaccgcaaagcattttcaggcaaagtcctggcaattgttcagtcaacaaaagaagcaggcga
aattacagttacagcaaaagcagatggcctgcaatcaagcacagttaaaattgcaacaacagcagttccgggaaca
agcacagaaaaaacagtccgcagcttttattacagccgcaactattatgtcaaaacaggcaacaaaccgattctgcc
gtcagatgttgaagttcgctattcagatggaacaagcgatagacaaaacgttacatgggatgcagtttcagatgatcaa
attgcaaaagcaggctcattttcagttgcaggcacagttgcaggccaaaaaattagcgttcgcgtcacaatgattgatg
aaattggcgcactgctgaattattcagcaagcacaccggttggcacaccggcagttcttccgggatcaagaccggca
gtcctgccggatggcacagtcacatcagcaaattttgcagtccattggacaaaaccggcagatacagtctataataca
gcaggcacagtcaaagtaccgggaacagcaacagtttttggcaaagaatttaaagtcacagcgacaattagagttc
aaagaagccaagttacaattggctcatcagtttcaggaaatgcactgagactgacacaaaatattccggcagataaa
caatcagatacactggatgcgattaaagatggctcaacaacagttgatgcaaatacaggcggaggcgcaaatccgt
cagcatggacaaattgggcatattcaaaagcaggccataacacagcggaaattacatttgaatatgcgacagaaca
acaactgggccagatcgtcatgtatttttttcgcgatagcaatgcagttagatttccggatgctggcaaaacaaaaattca
gatcagcgcagatggcaaaaattggacagatctggcagcaacagaaacaattgcagcgcaagaatcaagcgata
gagtcaaaccgtatacatatgattttgcaccggttggcgcaacatttgttaaagtgacagtcacaaacgcagatacaac
aacaccgtcaggcgttgtttgcgcaggcctgacagaaattgaactgaaaacagcgaca
SEQ ID NO: 14 nucleotide sequence encoding BIF_1326
gttgaagatgcaacaagaagcgatagcacaacacaaatgtcatcaacaccggaagttgtttattcatcagcggtcgat
agcaaacaaaatcgcacaagcgattttgatgcgaactggaaatttatgctgtcagatagcgttcaagcacaagatccg
gcatttgatgattcagcatggcaacaagttgatctgccgcatgattatagcatcacacagaaatatagccaaagcaatg
aagcagaatcagcatatcttccgggaggcacaggctggtatagaaaaagctttacaattgatagagatctggcaggc
aaacgcattgcgattaattttgatggcgtctatatgaatgcaacagtctggtttaatggcgttaaactgggcacacatccg
tatggctattcaccgttttcatttgatctgacaggcaatgcaaaatttggcggagaaaacacaattgtcgtcaaagttgaa
aatagactgccgtcatcaagatggtattcaggcagcggcatttatagagatgttacactgacagttacagatggcgttc
atgttggcaataatggcgtcgcaattaaaacaccgtcactggcaacacaaaatggcggagatgtcacaatgaacctg
acaacaaaagtcgcgaatgatacagaagcagcagcgaacattacactgaaacagacagtttttccgaaaggcgga
aaaacggatgcagcaattggcacagttacaacagcatcaaaatcaattgcagcaggcgcatcagcagatgttacaa
gcacaattacagcagcaagcccgaaactgtggtcaattaaaaacccgaacctgtatacagttagaacagaagttctg
aacggaggcaaagttctggatacatatgatacagaatatggctttcgctggacaggctttgatgcaacatcaggcttttc
actgaatggcgaaaaagtcaaactgaaaggcgttagcatgcatcatgatcaaggctcacttggcgcagttgcaaata
gacgcgcaattgaaagacaagtcgaaatcctgcaaaaaatgggcgtcaatagcattcgcacaacacataatccgg
cagcaaaagcactgattgatgtctgcaatgaaaaaggcgttctggttgtcgaagaagtctttgatatgtggaaccgcag
caaaaatggcaacacggaagattatggcaaatggtttggccaagcaattgcaggcgataatgcagttctgggaggc
gataaagatgaaacatgggcgaaatttgatcttacatcaacaattaaccgcgatagaaatgcaccgtcagttattatgt
ggtcactgggcaatgaaatgatggaaggcatttcaggctcagtttcaggctttccggcaacatcagcaaaactggttgc
atggacaaaagcagcagattcaacaagaccgatgacatatggcgataacaaaattaaagcgaactggaacgaat
caaatacaatgggcgataatctgacagcaaatggcggagttgttggcacaaattattcagatggcgcaaactatgata
aaattcgtacaacacatccgtcatgggcaatttatggctcagaaacagcatcagcgattaatagccgtggcatttataat
agaacaacaggcggagcacaatcatcagataaacagctgacaagctatgataattcagcagttggctggggagca
gttgcatcatcagcatggtatgatgttgttcagagagattttgtcgcaggcacatatgtttggacaggatttgattatctggg
cgaaccgacaccgtggaatggcacaggctcaggcgcagttggctcatggccgtcaccgaaaaatagctattttggca
tcgttgatacagcaggctttccgaaagatacatattatttttatcagagccagtggaatgatgatgttcatacactgcatatt
cttccggcatggaatgaaaatgttgttgcaaaaggctcaggcaataatgttccggttgtcgtttatacagatgcagcgaa
agtgaaactgtattttacaccgaaaggctcaacagaaaaaagactgatcggcgaaaaatcatttacaaaaaaaaca
acagcggcaggctatacatatcaagtctatgaaggcagcgataaagattcaacagcgcataaaaacatgtatctgac
atggaatgttccgtgggcagaaggcacaatttcagcggaagcgtatgatgaaaataatcgcctgattccggaaggca
gcacagaaggcaacgcatcagttacaacaacaggcaaagcagcaaaactgaaagcagatgcggatcgcaaaa
caattacagcggatggcaaagatctgtcatatattgaagtcgatgtcacagatgcaaatggccatattgttccggatgc
agcaaatagagtcacatttgatgttaaaggcgcaggcaaactggttggcgttgataatggctcatcaccggatcatgat
tcatatcaagcggataaccgcaaagcattttcaggcaaagtcctggcaattgttcagtcaacaaaagaagcaggcga
aattacagttacagcaaaagcagatggcctgcaatcaagcacagttaaaattgcaacaacagcagttccgggaaca
agcacagaaaaaacagtccgcagcttttattacagccgcaactattatgtcaaaacaggcaacaaaccgattctgcc
gtcagatgttgaagttcgctattcagatggaacaagcgatagacaaaacgttacatgggatgcagtttcagatgatcaa
attgcaaaagcaggctcattttcagttgcaggcacagttgcaggccaaaaaattagcgttcgcgtcacaatgattgatg
aaattggcgcactgctgaattattcagcaagcacaccggttggcacaccggcagttcttccgggatcaagaccggca
gtcctgccggatggcacagtcacatcagcaaattttgcagtccattggacaaaaccggcagatacagtctataataca
gcaggcacagtcaaagtaccgggaacagcaacagttfttggcaaagaatttaaagtcacagcgacaattagagttc
aaagaagccaagttacaattggctcatcagtttcaggaaatgcactgagactgacacaaaatattccggcagataaa
caatcagatacactggatgcgattaaagatggctcaacaacagttgatgcaaatacaggcggaggcgcaaatccgt
cagcatggacaaattgggcatattcaaaagcaggccataacacagcggaaattacatttgaatatgcgacagaaca
acaactgggccagatcgtcatgtatttftttcgcgatagcaatgcagttagatttccggatgctggcaaaacaaaaattca
gatcagcgcagatggcaaaaattggacagatctggcagcaacagaaacaattgcagcgcaagaatcaagcgata
gagtcaaaccgtatacatatgattttgcaccggttggcgcaacatttgttaaagtgacagtcacaaacgcagatacaac
aacaccgtcaggcgttgfttgcgcaggcctgacagaaattgaactgaaaacagcgacaagcaaatttgtcacaaata
catcagcagcactgtcatcacttacagtcaatggcacaaaagtttcagattcagttctggcagcaggctcatataacac
accggcaattatcgcagatgttaaagcggaaggcgaaggcaatgcaagcgttacagtccttccggcacatgataatg
ttattcgcgtcattacagaaagcgaagatcatgtcacacgcaaaacatttacaatcaacctgggcacagaacaagaa
ttt
>SEQ ID NO: 15 forward primer for generation of BIF variants
GGGGTAACTAGTGGAAGATGCAACAAGAAG
>SEQ ID NO: 16 reverse primer for BIF917
GCGCTTAATTAATTATGTTTTTTCTGTGCTTGTTC
>SEQ ID NO: 17 reverse primer for BIF995
GCGCTTAATTAATTACAGTGCGCCAATTTCATCAATCA
>SEQ ID NO: 18 reverse primer for BIF1068
GCGCTTAATTAATTATTGAACTCTAATTGTCGCTG
>SEQ ID NO: 19 reverse primer for BIF1241
GCGCTTAATTAATTATGTCGCTGTTTTCAGTTCAAT
>SEQ ID NO: 20 reverse primer for BIF1326
GCGCTTAATTAATTAAAATTCTTGTTCTGTGCCCA
>SEQ ID NO: 21 reverse primer for BIF1478
GCGCTTAATTAATTATCTCAGTCTAATTTCGCTTGCGC
>SEQ ID NO: 22 Bifidobacterium bifidum BIF1750
vedatrsdsttqmsstpevvyssavdskqnrtsdfdanwkfmlsdsvqaqdpafddsawqqvdlphdysitqkys
qsneaesaylpggtgwyrksftidrdlagkriainfdgvymnatvwfngvklgthpygyspfsfdltgnakfggentivv
kvenrlpssrwysgsgiyrdvtltvtdgvhvgnngvaiktpslatqnggdytmnlttkvandteaaanitlkqtvfpkggk
tdaaigtvttasksiaagasadvtstitaaspklwsiknpnlytvrtevlnggkvldtydteygfrwtgfdatsgfslngekv
klkgvsmhhdqgslgavanrraierqveilqkmgvnsirtthnpaakalidvcnekgvlvveevfdmwnrskngnte
dygkwfgqaiagdnavlggdkdetwakfdltstinrdrnapsvimwslgnemmegisgsvsgfpatsaklvawtka
adstrpmtygdnkikanwnesntmgdnltanggvvgtnysdganydkirtthpswaiygsetasainsrgiynrttgg
aqssdkqltsydnsavgwgavassawydvvqrdfvagtyvwtgfdylgeptpwngtgsgavgswpspknsyfgiv
dtagfpkdtyyfyqsqwnddvhtlhilpawnenvvakgsgnnvpvvvytdaakvklyftpkgstekrligeksftkktta
agytyqvyegsdkdstahknmyltwnvpwaegtisaeaydennrlipegstegnasvtttgkaaklkadadrktitad
gkdlsyievdvtdanghivpdaanrvtfdvkgagklvgvdngsspdhdsyqadnrkafsgkvlaivqstkeageitvt
akadglqsstvkiattavpgtstektvrsfyysrnyyvktgnkpilpsdvevrysdgtsdrqnvtwdavsddqiakagsf
svagtvagqkisvrvtmideigallnysastpvgtpavlpgsrpavlpdgtvtsanfavhwtkpadtvyntagtvkvpgt
atvfgkefkvtatirvqrsqvtigssvsgnalrltqnipadkqsdtldaikdgsttvdantggganpsawtnwayskagh
ntaeitfeyateqqlgqivmyffrdsnavrfpdagktkiqisadgknwtdlaatetiaaqessdrvkpytydfapvgatfv
kvtvtnadtttpsgvvcaglteielktatskfvtntsaalssltvngtkvsdsvlaagsyntpaiiadvkaegegnasvtvlp
andnvirvitesedhvtrktftinlgteqefpadsderdypaadmtvtvgseqtsgtategpkkfavdgntstywhsnwt
pttvndlwiafelqkptkldalrylprpagskngsvteykvqvsddgtnwtdagsgtwttdygwklaefnqpvttkhvrlk
avhtyadsgndkfmsaseirlrkavdttdisgatvtvpakltvdrvdadhpatfatkdvtvtlgdatlrygvdylldyagnt
avgkatvtvrgidkysgtvaktftielknapapeptltsvsvktkpskltyvvgdafdpaglvlqhdrqadrppqplvgeq
adergltcgtrcdrveqlrkhenreahrtgldhlefvgaadgavgeqatfkvhvhadqgdgrhddaderdidphvpvd
havgelaraachhviglrvdthrlkasgfqipaddmaeidritgfhrferhvg
>SEQ ID NO: 23 The signal sequence of extracellular lactase from
Bifidobacterium bifidum DSM20215
Vrskklwisllfalaliftmafgstssaqa

1. A method of generating a saccharide containing a galactose moiety and a fructose moiety, wherein:
(a) the galactose moiety is linked to the fructose moiety; or
(b) the galactose moiety and the fructose moiety are separated by at least one monosaccharide moiety other than galactose or fructose;
the method comprising:
contacting a first saccharide, said first saccharide containing a galactose moiety, with a second saccharide, said second saccharide containing a fructose moiety, the first and the second saccharides being different,
in the presence of an enzyme capable of catalysing the transfer of a galactose moiety to the second saccharide containing the fructose moiety,
the method being carried out at a pH of 5.5 to 9.5;
provided that
(i) when the galactose moiety is linked to the fructose moiety, the concentration of the first saccharide is less than 0.43 mol/L and the concentration of the second saccharide is less than 0.5 mol/L; and
(ii) when the galactose moiety and the fructose moiety are separated by at least one monosaccharide moiety other than galactose or fructose, the concentration of the first saccharide and/or the concentration of the second saccharide is less than 0.5 mol/L.

2. A method of generating a saccharide containing a galactose moiety and a fructose moiety, wherein:
(a) the galactose moiety is linked to the fructose moiety; or
(b) the galactose moiety and the fructose moiety are separated by at least one monosaccharide moiety other than galactose or fructose; the method comprising:
contacting a first saccharide, said first saccharide containing a galactose moiety, with a second saccharide, said second saccharide containing a fructose moiety, the first and the second saccharides being different,
in the presence of an enzyme capable of catalysing the transfer of a galactose moiety to the second saccharide containing the fructose moiety,
wherein the enzyme is selected from the group consisting of:
a) a polypeptide comprising an amino acid sequence having at least 90% sequence identity with SEQ ID NO: 1, wherein said polypeptide consists of at most 980 amino acid residues;
b) a polypeptide encoded by a polynucleotide that hybridizes under at least low stringency conditions with:
i) the nucleic acid sequence comprised in SEQ ID NO: 9, encoding the polypeptide of SEQ ID NO: 1; or
ii) the complementary strand of i).

3. A method of generating a saccharide in which a galactose moiety is linked to a fructose moiety, the method comprising:
contacting a first saccharide, said first saccharide containing a galactose moiety, with a second saccharide, said second saccharide containing a fructose moiety, the first and the second saccharides being different,
in the presence of an enzyme capable of catalysing the transfer of a galactose moiety to the second saccharide containing the fructose moiety,
the method being carried out at a pH of 5.5 to 9.5;
wherein:
the concentration of the first saccharide is less than 0.43 mol/L; and
the concentration of the second saccharide is less than 0.8 mol/L.

4. The method of claim 3, wherein the first saccharide is lactose or a galactooligosaccharide.

5. The method of claim 4, wherein the first saccharide is lactose.

6. The method of claim 3 or claim 4, wherein the second saccharide is fructose.

7. The method of claim 3, wherein the first saccharide is lactose and the second saccharide is fructose, so that the saccharide generated is lactulose.

8. The method of claim 3, wherein the first saccharide is lactose and the second saccharide is lactulose.

9. The method of any one of claims 3 to 8, wherein the concentration of the first saccharide is from 0.088 to 0.380 mol/L.

10. The method of claim 5, wherein the concentration of the lactose is from 40 to 100 g/L.

11. The method of any one of claims 3 to 10, wherein the concentration of the second saccharide is 0.278 to 0.444 mol/L.

12. The method of claim 6, wherein the concentration of the fructose is from 50 to 80 g/L.

13. The method of any preceding claim, wherein the enzyme is a β-galactosidase.

14. The method of any preceding claim, wherein the enzyme is classified in Enzyme Classification (E.C.) 3.2.1.23.

15. The method of any preceding claim, wherein the enzyme is of bacterial origin or fungal origin.

16. The method of claim 15, wherein the enzyme is of Bifidobacteria origin.

17. The method of any preceding claim, wherein the enzyme is selected from the group consisting of:
a) a polypeptide having transgalactosylating activity and comprising an amino acid sequence having at least 90% sequence identity with SEQ ID NO: 1, wherein said polypeptide consists of at most 980 amino acid residues;
b) a polypeptide encoded by a polynucleotide that hybridizes under at least low stringency conditions with:
i) the nucleic acid sequence comprised in SEQ ID NO: 9, encoding the polypeptide of SEQ ID NO: 1; or
ii) the complementary strand of i).

18. A method of generating a saccharide in which a galactose moiety is linked to a fructose moiety, the method comprising:
contacting a first saccharide, said first saccharide containing a galactose moiety, with a second saccharide, said second saccharide containing a fructose moiety, the first and the second saccharides being different,
in the presence of an enzyme capable of catalysing the transfer of a galactose moiety to the second saccharide containing the fructose moiety,
wherein the enzyme is selected from the group consisting of:
a) a polypeptide comprising an amino acid sequence having at least 90% sequence identity with SEQ ID NO: 1, wherein said polypeptide consists of at most 980 amino acid residues;
b) a polypeptide encoded by a polynucleotide that hybridizes under at least low stringency conditions with:
i) the nucleic acid sequence comprised in SEQ ID NO: 9, encoding the polypeptide of SEQ ID NO: 1; or
ii) the complementary strand of i).

19. The method of any one of claims 3 to 18, carried out at a temperature of 0 to 10° C.

20. The method of any one of claims 3 to 18, carried out at a temperature of 45 to 60° C.

21. The method of any preceding claim, carried out in situ in a food composition.

22. The method of claim 21, wherein the food composition is a dairy composition.

23. The method of claim 22, wherein lactose is present as an initial component of the dairy composition.

24. The method of claim 22 or claim 23, wherein lactose is added to the dairy composition.

25. The method of any preceding claim, wherein the yield of lactulose is at least 10%, such as at least 12%, such as at least 15%, such as at least 18%, such as at least 20%, such as at least 22%, such as at least 25%, calculated by weight based on the total weight of lactose and fructose used as starting material.

26. A lactulose-containing composition obtainable by the method of any preceding claim.

27. A method of generating a saccharide containing a galactose moiety and a fructose moiety, the galactose moiety and the fructose moiety being separated by at least one monosaccharide moiety other than galactose or fructose, the method comprising:
contacting a first saccharide, said first saccharide containing a galactose moiety, with a second saccharide, said second saccharide containing a fructose moiety, the first and the second saccharides being different,
in the presence of an enzyme capable of catalysing the transfer of a galactose moiety to the second saccharide containing the fructose moiety,
the method being carried out at a pH of 5.5 to 9.5;
wherein the concentration of the first saccharide and/or the concentration of the second saccharide is less than 0.5 mol/L.

28. The method of claim 27, wherein the first saccharide is lactose or a galactooligosaccharide.

29. The method of claim 28, wherein the first saccharide is lactose.

30. The method of claim 27 or claim 28, wherein the second saccharide is sucrose.

31. The method of claim 27, wherein the first saccharide is lactose and the second saccharide is sucrose, so that the saccharide generated is lactosucrose.

32. The method of any one of claims 27 to 31, wherein the concentration of the first saccharide is from 0.01 to 0.25 mol/L.

33. The method of claim 29, wherein the concentration of the lactose is from 0.1 to 0.2 mol/L.

34. The method of any one of claims 27 to 33, wherein the concentration of the second saccharide is 0.01 to 0.35 mol/L.

35. The method of claim 30, wherein the concentration of the sucrose is from 0.1 to 0.35 mol/L.

36. The method of any one of claims 27 to 35, wherein the enzyme is a β-galactosidase.

37. The method of any one of claims 27 to 36, wherein the enzyme is classified in Enzyme Classification (E.C.) 3.2.1.23.

38. The method of any one of claims 27 to 37, wherein the enzyme is of bacterial origin or fungal origin.

39. The method of claim 38, wherein the enzyme is of Bifidobacteria origin.

40. The method of any one of claims 27 to 39, wherein the enzyme is selected from the group consisting of:
a) a polypeptide having transgalactosylating activity and comprising an amino acid sequence having at least 90% sequence identity with SEQ ID NO: 1, wherein said polypeptide consists of at most 980 amino acid residues;
b) a polypeptide encoded by a polynucleotide that hybridizes under at least low stringency conditions with:
i) the nucleic acid sequence comprised in SEQ ID NO: 9, encoding the polypeptide of SEQ ID NO: 1; or
ii) the complementary strand of i).

41. A method of generating a saccharide containing a galactose moiety and a fructose moiety, the galactose moiety and the fructose moiety being separated by at least one monosaccharide moiety other than galactose or fructose, the method comprising:
contacting a first saccharide, said first saccharide containing a galactose moiety, with a second saccharide, said second saccharide containing a fructose moiety, the first and the second saccharides being different,
in the presence of an enzyme capable of catalysing the transfer of a galactose moiety to the second saccharide containing the fructose moiety,
wherein the enzyme is selected from the group consisting of:
a) a polypeptide comprising an amino acid sequence having at least 90% sequence identity with SEQ ID NO: 1, wherein said polypeptide consists of at most 980 amino acid residues;
b) a polypeptide encoded by a polynucleotide that hybridizes under at least low stringency conditions with:
i) the nucleic acid sequence comprised in SEQ ID NO: 9, encoding the polypeptide of SEQ ID NO: 1; or
ii) the complementary strand of i).

42. The method of any one of claims 27 to 41, carried out at a temperature of 30 to 70° C.

43. The method of any one of claims 27 to 42, carried out in situ in a food composition.

44. The method of claim 43, wherein the food composition is a dairy composition.

45. The method of claim 44, wherein lactose is present as an initial component of the dairy composition.

46. The method of claim 44 or claim 45, wherein lactose is added to the dairy composition.

47. A lactosucrose-containing composition obtainable by the method of any preceding claim.
